(19) United States
(12) Patent Application Publication
(10) Pub. No.: US 2005/0054637 A1
(43) Pub. Date: Mar. 10, 2005

(54) Substituted piperazine cyclohexane carboxilic acid amides and the use thereof

(76) Inventors: Erwin Bischoff, Wuppertal (DE); Thomas Krahn, Hagen (DE); Holger Paulsen, Wuppertal (DE); Joachim Schuhmacher, Wuppertal (DE); Henning Steinhagen, Sulzbach (DE); Wolfgang Thielemann, Wuppertal (DE)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/492,337

(22) PCT Filed: Oct. 1, 2002
(86) PCT No.: PCT/EP02/10978

(30) Foreign Application Priority Data
Oct. 11, 2001 (DE) 101 50 310.5

(51) Int. Cl. C07D 417/02; C07D 413/02; C07D 43/02; A61K 31/496; A61K 31/495

(52) U.S. Cl. 514/227.8; 514/235.2; 514/252.13; 514/252.14; 514/253.01; 514/254.01; 514/255.01; 544/60; 544/114; 544/295; 544/360; 544/373

(57) ABSTRACT

The present invention relates to substituted piperazinecyclohexanecarboxamides of the formula (I) 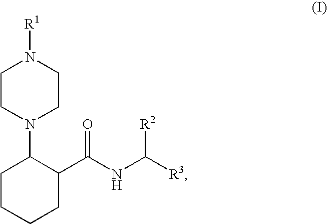 to processes for their preparation and to their use in pharmaceuticals, in particular for the prophylaxis and/or treatment of cardiovascular disorders.

The present invention relates to substituted piperazinecyclohexanecarboxamides, to a process for their preparation and to their use in pharmaceuticals, in particular for the prophylaxis and/or treatment of cardiovascular disorders.

Adenosine is an endogenic effector with cell-protective activity, in particular under cell-damaging conditions with limited oxygen supply, such as, for example, in the case of ischaemia. Adenosine is a highly effective vasodilator. It increases ischaemic “preconditioning” (R. Strasser, A. Vogt, W. Scharper, Z. Kardiologie 85, 1996, 79-89) and can promote the growth of collateral vessels. It is released under hypoxic conditions, for example in the case of cardiac or peripheral occlusive diseases (W. Makarewicz “Purine and Pyrimidine Metabolism in Man”, Plenum Press New York, 11, 1998, 351-357). Accordingly, adenosine protects against the effects of disorders caused by ischaemia, for example by increasing the coronary or peripheral circulation by vasodilation, by inhibiting platelet aggregation and by stimulating angiogenesis. Compared to systemically administered adenosine, the adenosine-uptake inhibitors have the advantage of selectivity for ischaemia. Moreover, systemically administered adenosine has a very short half-life. Systemically administered adenosine causes a strong systemic lowering of the blood pressure, which is undesirable, since circulation into the ischaemic regions may be reduced even further (“steal phenomenon”, L. C. Becker, Circulation 57, 1978, 1103-1110). The adenosine-uptake inhibitor increases the effect of the adenosine which is formed locally owing to the ischaemia and thus only dilates the vessels in the ischaemic regions. Accordingly, orally or intravenously administered adenosine-uptake inhibitors can be used for preventing and/or treating ischaemic disorders.

Furthermore, there have been various indications of a neuroprotective, anticonvulsive, analgesic and sleep-inducing potential of adenosine-uptake inhibitors, since they increase the intrinsic effects of adenosine by inhibiting its cellular re-uptake (K. A. Rudolphi et al., Cerebrovascular and Brain. Metabolism Reviews 4, 1992, 364-369; T. F. Murray et al., Drug Dev. Res. 28, 1993, 410-415; T. Porkka-Heiskanen et al., Science 276, 1997, 1265-1268; ‘Adenosine in the Nervous System’, Ed.: Trevor Stone, Academic Press Ltd. 1991, 217-227; M. P. DeNinno, Annual Reports in Medicinal Chemistry 33, 1998, 111-120).

Phenylcyclohexanecarboxamides acting as adenosine-uptake inhibitors have been described, for example, in WO 00/073,274.

Accordingly, it is an object of the present invention to provide novel substances for the prophylaxis and/or treatment of cardiovascular disorders.

The present invention relates to compounds of the formula (I) 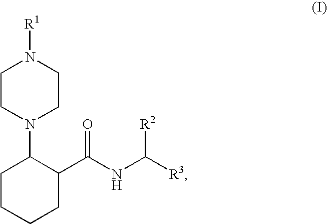
in which
R¹ represents a group of the formula *C(═O)—R⁴, *(CH₂)ₐR⁴, *SO₂—R⁴, *C(═O) NR⁵R⁶ or *C(═O)—OR⁷ bedeutet,
   in which
   * represents the point of attachment,
   a represents. 0, 1, 2 or 3,
   R⁴ represents (C₁-C₆)-alkyl, (C₃-C₈)-cycloalkyl, which is optionally substituted by (C₁-C₆)-alkyl or hydroxyl, (C₆-C₁₀)-aryl or 5- to 10-membered heteroaryl having up to three heteroatoms from the group consisting of N, O and S,
      where aryl and heteroaryl for their part may be substituted up to three times independently of one another by halogen, trifluoromethyl, trifluoromethoxy, cyano, carboxyl, nitro, hydroxyl, sulphamoyl, (C₁-C₆)-alkoxy, (C₁-C₆)-alkoxycarbonyl, amino, mono- or di-(C₁-C₆)-alkylamino, (C₁-C₄)-alkylcarbonylamino, (C₃-C₈)-cycloalkyl, (C₆-C₁₀)-aryl, 5- or 6-membered heteroaryl having up to three heteroatoms from the group consisting of N, O and S, 5- to 7-membered heterocyclyl having up to two heteroatoms from the group consisting of N, O and S, where N is substituted by hydrogen, (C₁-C₄)-alkyl or (C₃-C₇)-cyclo-alkyl, or
      (C₁-C₆)-alkyl, whose chain may be interrupted by an oxygen atom or a sulphur atom or by an NH group and which for its part may be substituted by hydroxyl, mono- or di-(C₁-C₆)-alkylamino, phenyl or 5- to 7-membered heterocyclyl having up to two heteroatoms from the group consisting of N, O and S, where N is substituted by hydrogen, (C₁-C₄)-alkyl or (C₃-C₇)-cycloalkyl,
   R⁵ and R⁶ independently of one another represent hydrogen, (C₆-C₁₀)-aryl or 5- to 10-membered heteroaryl having up to three heteroatoms from the group consisting of N, O and S, where aryl and heteroaryl for their part may be substituted up to three times independently of one another by halogen, trifluoromethyl, trifluoromethoxy, cyano, nitro, hydroxyl, amino, (C₁-C₆)-alkyl or (C₁-C₆)-alkoxy,
      adamantyl, (C₁-C₈)-alkyl, whose chain may be interrupted by one or two oxygen atoms and which may be substituted up to three times independently of one another by hydroxyl, phenyl, trifluoromethyl, (C₃-C₈)-cycloalkyl, (C₁-C₆)-alkoxy, mono- or di-(C₁-C₆)-alkylamino, 5- or 6-membered heterocyclyl having up to three heteroatoms from the group consisting of N, O and S or by 5- to 10-membered heteroaryl having up to three heteroatoms from the group consisting of N, O and S,
      (C₃-C₈)-cycloalkyl, which may be substituted up to three times independently of one another by (C₁-C₄)-alkyl, hydroxyl or oxo, or
      -5- or 6-membered heterocyclyl having up to two heteroatoms from the group consisting of N, O and S, where N is substituted by hydrogen or (C₁-C₄)-alkyl,
   or
   R⁵ and R⁶ together with the nitrogen atom to which they are attached form a 4- to 7-membered saturated heterocycle which may contain up to two further heteroatoms from the group consisting of N, O and S and which is optionally substituted by hydroxyl, oxo or (C₁-C₆)-alkyl which for its part may be substituted by hydroxyl,
   R⁷ represents (C₆-C₁₀)-aryl or 5- to 10-membered heteroaryl having up to three heteroatoms from the group consisting of N, O and S, where aryl and heteroaryl for their part may be substituted up to three times independently of one another by halogen, trifluoromethyl, trifluoromethoxy, cyano, nitro, hydroxyl, amino, (C₁-C₆)-alkyl or (C₁-C₆)-alkoxy,
      adamantyl, (C₁-C₈)-alkyl whose chain may be interrupted by one or two oxygen atoms and which may be substituted up to three times independently of one another by hydroxyl, phenyl which for its part may be substituted by nitro, halogen, trifluoromethyl, trifluoromethoxy, (C₁-C₆)-alkyl or cyano, trifluoromethyl, (C₃-C₈)-cycloalkyl, (C₁-C₆)-alkoxy, mono- or di-(C₁-C₆)-alkylamino, 5- or 6-membered heterocyclyl having up to three heteroatoms from the group consisting of N, O and S or by 5- to 10-membered heteroaryl having up to three heteroatoms from the group consisting of N, O and S, (C₃-C₈)-cycloalkyl which may be substituted up to three times independently of one another by (C₁-C₄)-alkyl, hydroxyl or oxo, or
      5- or 6-membered heterocyclyl having up to two heteroatoms from the group consisting of N, O and S, where N is substituted by hydrogen or (C₁-C₄)-alkyl,
R² represents (C₁-C₈)-alkyl whose chain may be interrupted by a sulphur atom or oxygen atom or by an S(O) or SO₂ group, phenyl, benzyl or 5- or 6-membered heteroaryl having up to two heteroatoms from the group consisting of N, O and S, where phenyl, benzyl and heteroaryl for their part may be substituted up to three times independently of one another by halogen, trifluoromethyl, trifluoromethoxy, cyano, nitro, hydroxyl, amino, (C₁-C₆)-alkyl or (C₁-C₆)-alkoxy,
and
R³ represents a group of the formula *CH₂—OH or *C(O)NR⁸R⁹,
   in which
   * represents the point of attachment,
   R⁸ and R⁹ independently of one another represent hydrogen or (C₁-C₆)-alkyl, or
R² and R³ together with the CH group to which they are attached represent a group of the formula 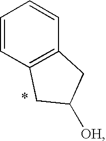
   in which
   * represents the point of attachment,
      and their salts, hydrates, hydrates of the salts and solvates.

Salts of the compounds according to the invention are physiologically acceptable salts of the substances according to the invention with mineral acids, carboxylic acids or sulphonic acids. Particular preference is given, for example, to salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, propionic acid, lactic acid, tartaric acid, citric acid, fumaric acid, maleic acid or benzoic acid.

Salts can also be physiologically acceptable metal or ammonium salts of the compounds according to the invention. Particularly preferred are alkali metal salts (for example sodium salts or potassium salts), alkaline earth metal salts (for example magnesium salts or calcium salts), and also ammonium salts, which are derived from ammonia or organic amines, such as, for example, ethylamine, di- or triethylamine, di- or triethanolamine, dicyclohexylamine, dimethylaminoethanol, arginine, lysine, ethylenediamine or 2-phenylethylamine.

Depending on the substitution pattern, the compounds according to the invention can exist in stereoisomeric forms which are either like image and mirror image (enantiomers) or which are not like image and mirror image (diastereomers). The invention relates both to the enantiomers or diastereomers: and to their respective mixtures. The racemic forms, like the diastereomers, can be separated in a known manner into the stereoisomerically uniform components.

Moreover, the invention also includes prodrugs of the compounds according to the invention. According to the invention, prodrugs are those forms of the compounds of the above formula (I) which for their part may be biologically active or inactive, but which are: converted under physiological conditions (for example metabolically or solvolytically) into the corresponding biologically active form.

According to the invention, “hydrates” or “solvates” are those forms of the compounds of the formula (I) which, in solid or liquid state, form a molecular compound or a complex by hydration with water or coordination with solvent molecules. Examples of hydrates are sesquihydrates, monohydrates, dihydrates and trihydrates. Equally suitable are the hydrates or solvates of salts of the compounds according to the invention.,

Halogen represents fluorine, chlorine, bromine and iodine. Preference is given to chlorine or fluorine.

(C₁-C₈)-Alkyl represents a straight-chain or branched alkyl radical having 1 to 8 carbon atoms. Examples which may be mentioned are: methyl, ethyl, n-propyl, isopropyl, n-butyl, isobutyl, tert-butyl, n-pentyl, n-hexyl and n-octyl. The corresponding alkyl groups having fewer carbon atoms, such as, for example (C₁-C₆)-alkyl, (C₁-C₄)-alkyl and (C₁-C₃)-alkyl, are derived analogously from this definition. In general, (C₁-C₃)-alkyl is preferred.

The meaning of the corresponding component of other, more complex substituents, such as, for example, mono- or di-alkylamino or alkylcarbonylamino is also derived from this definition.

Mono- or di-(C₁-C₄)-alkylamino represents an amino group having one or two identical or different straight-chain or branched alkyl substituents of in each case 1 to 4 carbon atoms. Examples which may be mentioned are: methylamino, ethylamino, n-propylamino, isopropylamino, tert-butylamino, N,N-dimethylamino, N,N-diethylamino, N-ethyl-N-methylamino, N-methyl-N-n-propylamino, N-isopropyl-N-n-propylamino and N-t-butyl-N-methylamino.

(C₁-C₄)-Alkylcarbonylamino represents an alkylcarbonyl group which is attached via an amino group. Acetylamino and propanoylamino may be mentioned by way of example and by way of preference.

(C₃-C₈)-Cycloalkyl represents a cyclic alkyl radical having 3 to 8 carbon atoms. Examples which may be mentioned are: cyclopropyl, cyclobutyl, cyclopentyl, cyclohexyl, cycloheptyl and cyclooctyl. The corresponding cycloalkyl groups having fewer carbon atoms, such as, for example, (C₃-C₇)-cycloalkyl or (C₃-C₆)-cycloalkyl, are derived analogously from this definition. Preference is given to cyclopropyl, cyclopentyl and cyclohexyl.

(C₁-C₆)-Alkoxy represents a straight-chain or branched alkoxy radical having 1 to 6 carbon atoms. Examples which may be mentioned are: methoxy, ethoxy, n-propoxy, isopropoxy, n-butoxy, isobutoxy, tert-butoxy, n-pentoxy and n-hexoxy. The corresponding alkoxy groups having fewer carbon atoms, such as, for example, (C₁-C₄)-alkoxy or (C₁-C₃)-alkoxy, are derived analogously from this definition. In general, (C₁-C₃)-alkoxy is preferred.

(C₁-C₆)-alkoxycarbonyl represents a straight-chain or branched alkoxy radical having 1 to 6 carbon atoms which is attached via a carbonyl group. Preference is given to a straight-chain or branched alkoxycarbonyl radical having 1 to 4 carbon atoms. Examples which may be mentioned are: methoxycarbonyl, ethoxycarbonyl, n-propoxycarbonyl, isopropoxycarbonyl and t-butoxycarbonyl.

(C₆-C₁₀)-aryl represents an aromatic radical having 6 to 10 carbon atoms. Examples which may be mentioned are: phenyl and naphthyl.

5- to 10-membered heteroaryl having up to 3 heteroatoms from the group consisting of N, O and S represents a mono- or bicyclic, if appropriate benzo-fused, heterocycle (heteroaromatic) which is attached via a ring carbon atom of the heteroaromatic, if appropriate also via a ring nitrogen atom of the heteroaromatic. Examples which may be mentioned are: pyridyl, pyrimidyl, pyridazinyl, pyrazinyl, thienyl, furyl, pyrrolyl, pyrazolyl, imidazolyl, triazolyl, thiazolyl, oxazolyl, oxdiazolyl, isoxazolyl, benzofuranyl, benzothienyl or benzimidazolyl. The corresponding heteroaromatics having fewer heteroatoms, such as, for example, those having up to 2 heteroatoms from the group consisting of N, O and S are derived analogously from this definition. In general, preference is given to 5- or 6-membered aromatic heterocycles having up to 2 heteroatoms from the group consisting of N, O and S, such as, for example, pyridyl, pyrimidyl, pyridazinyl, furyl, imidazolyl and thienyl.

5- or 6-membered heterocyclyl having up to 3 heteroatoms from the group consisting of N, O and S represents a saturated or partially unsaturated heterocycle which is attached via a ring carbon atom or a ring nitrogen atom. Examples which may be mentioned are: tetrahydrofuryl, pyrrolidinyl, pyrrolinyl, dihydropyridinyl, piperidinyl, piperazinyl, morpholinyl, thiomorpholinyl. Preference is given to saturated heterocycles, in particular to piperidinyl, piperazinyl, morpholinyl and pyrrolidinyl.

The compounds of the formula (I) according to the invention can be present in at least eight different configurations, the four different configurations (Ia) to (Id) below being preferred:

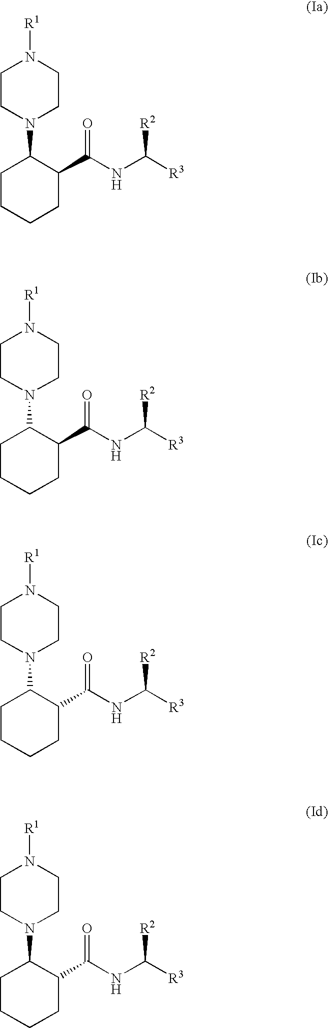

Particular preference is given to configuration (Id).

Preference is given to compounds of the formula (I),
in which
R¹ represents a group of the formula *C(═O)—R⁴, *(CH₂)ₐ—R⁴ or *C(═O)—OR⁷,
   in which
   * represents the point of attachment,
   a represents 1
   R⁴ represents (C₆-C₁₀)-aryl or 5- to 10-membered heteroaryl having up to three heteroatoms from the group consisting of N, O and S, where aryl and heteroaryl may be substituted up to three times independently of one another by halogen, trifluoromethyl, trifluoromethoxy, cyano, nitro, hydroxyl, amino, (C₁-C₆)-alkyl, (C₁-C₆)-alkylcarbonylamino or (C₁-C₆)-alkoxy,
   R⁷ represents phenyl which may be substituted up to three times independently of one another by halogen, trifluoromethyl, trifluoromethoxy, cyano, nitro, hydroxyl, amino, (C₁-C₆)-alkyl or (C₁-C₆)-alkoxy,
      methyl which may be substituted by phenyl or (C₃-C₈)-cycloalkyl, or (C₃-C₈)-cycloalkyl,
R² represents phenyl, benzyl or 5- or 6-membered heteroaryl having up to two heteroatoms from the group consisting of N, O and S, where phenyl, benzyl and heteroaryl for their part may be substituted up to three times independently of one another by halogen, hydroxyl, amino, (C₁-C₄)-alkyl or (C₁-C₄)-alkoxy, and
R³ represents a group of the formula *C(O)—NR⁸R⁹,
   in which
   * represents the point of attachment,
   R⁸ and R⁹ independently of one another represent hydrogen, methyl or ethyl,
      and their salts, hydrates, hydrates of the salts and solvates.

Particular preference is given to compounds of the formula (I)
in which
R¹ represents a group of the formula *C(═O)R⁴ or *(CH₂)ₐ—R⁴
   in which
   * represents the point of attachment,
   a represents 1,
   R⁴ represents (C₆-C₁₀)-aryl or 5- to 10-membered heteroaryl having up to three heteroatoms from the group consisting of N, O and S, where aryl and heteroaryl may be substituted up to three times independently of one another by halogen, trifluoromethyl, trifluoromethoxy, cyano, nitro, hydroxyl, amino, (C₁-C₆)-alkyl, (C₁-C₆)-alkylcarbonylamino or (C₁-C₆)-alkoxy,
R² represents phenyl, benzyl or 5- or 6-membered heteroaryl having up to two heteroatoms from the group consisting of N, O and S, where phenyl, benzyl and heteroaryl for their part may be substituted up to three times independently of one another by halogen, hydroxyl, amino, (C₁-C₄)-alkyl or (C₁-C₄)-alkoxy, and
R³ represents a group of the formula *C(O)—NR⁸R⁹,
   in which
   * represents the point of attachment,
   R⁸ and R⁹ independently of one another represent hydrogen or methyl,
      and their salts, hydrates, hydrates of the salts and solvates.

Very particular preference is given to compounds of the formula (I)
in which
R¹ represents a group of the formula *C(═O)—R⁴,
   in which
   * represents the point of attachment,
   R⁴ represents phenyl, naphthyl, indolyl, indazolyl, benzimidazolyl, benzisothiazolyl, pyrrolyl, furyl, thienyl, quinolinyl, isoquinolinyl, pyrazolyl, piperonyl, pyridinyl, pyrazinyl or pyridazinyl which for their part may be substituted up to two times independently of one another by fluorine, chlorine, trifluoromethyl, trifluoromethoxy, cyano, nitro, hydroxyl, acetylamino, methyl, ethyl, n-propyl, isopropyl, methoxy, ethoxy, n-propoxy or isopropoxy,
R² represents phenyl which may optionally be substituted by fluorine in the para position to the point of attachment, or pyridyl, and
R³ represents a group of the formula *C(O)—NR⁸R⁹,
   in which
   * represents the point of attachment,
   R¹ and R⁹ represent hydrogen,
      and their salts, hydrates, hydrates of the salts and solvates.

Especially very particularly preferred are the compounds of the following structures:
(1R,2R)-N-[(1S)-2-amino-2-oxo-1-phenylethyl]-2-(4-benzoyl-1-piperazinyl)cyclo-hexanecarboxamide 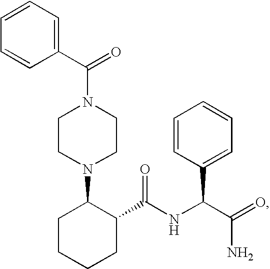
(1R,2R)-N-[(1S)-2-amino-2-oxo-1-(4-fluorophenyl)ethyl]-2-(4-benzoyl-1-piperazinyl)cyclohexanecarboxamide 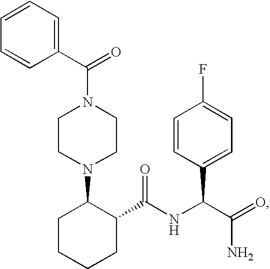
(1R,2R)-N-[(1S)-2-amino-2-oxo-1-phenylethyl]-2-[4-(1H-indazol-3-ylcarbonyl) 1-piperazinyl]cyclohexanecarboxamide 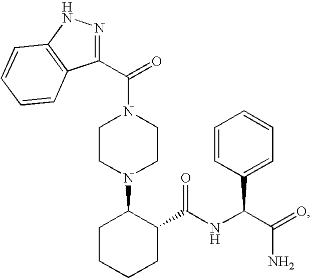
(1R,2R)-N-[(1S)-2-amino-2-oxo-1-phenylethyl]-2-[4-(2,4-difluorobenzoyl)-1-piperazinyl)cyclohexanecarboxamide 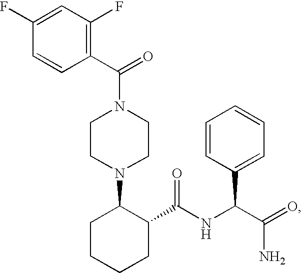
(1R,2R)-N-[(1S)-2-amino-2-oxo-1-phenylethyl]-2-{4-[(5-methyl-2-thienyl)carbonyl]-1-piperazinyl}cyclohexanecarboxamide 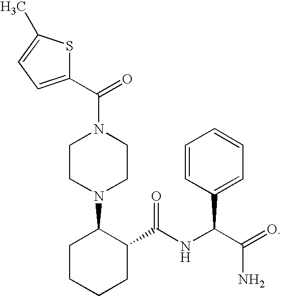
(1R,2R)-N-[(1S)-2-amino-2-oxo-1-phenylethyl]-2-{4-[(2-pyrrolyl)carbonyl]-1-piperazinyl}cyclohexanecarboxamide 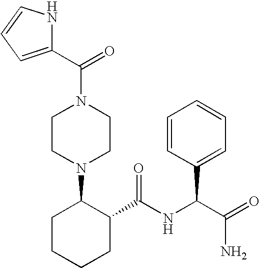
and their salts, hydrates, hydrates of the salts and solvates.

The present invention relates to compounds of the formula (I)
in which
R¹ represents a group of the formula *C(═O)—R⁴, *(CH₂)ₐ—R⁴, *SO₂—R⁴, *C(═O) NR⁵R⁶ or *C(═O)—OR⁷,
   in which
   * represents the point of attachment,
   a represents 0, 1, 2 or 3,
   R⁴ represents (C₃-C₈)-cycloalkyl, (C₆-C₁₀)-aryl or. 5- to 10-membered heteroaryl having up to three heteroatoms from the group consisting of N, O and S,
      where aryl and heteroaryl for their part may be substituted up to three times independently of one another by halogen, trifluoromethyl, trifluoromethoxy, cyano, carboxyl, nitro, hydroxyl, (C₁-C₆)-alkoxy, (C₁-C₆)-alkoxycarbonyl, amino, mono- or di-(C₁-C₆)-alkylamino, (C₃-C₈)-cycloalkyl, (C₆-C₁₀)-aryl, 5- or 6-membered heteroaryl having up to three heteroatoms from the group consisting of N, O and S, 5- to 7-membered heterocyclyl having up to two heteroatoms from the group consisting of N, O and S, where N is substituted by hydrogen, (C₁-C₄)-alkyl or (C₃-C₇)-cycloalkyl, or
         (C₁-C₆)-alkyl whose chain may be interrupted by an oxygen atom or a sulphur atom or by an NH group and which for its part may be substituted by hydroxyl, mono- or di-(C₁-C₆)-alkylamino or 5- to 7-membered heterocyclyl having up to two heteroatoms from the group consisting of N, O and S, where N is substituted by hydrogen, (C₁-C₄)-alkyl or (C₃-C₇)-cycloalkyl,
   R⁵ and R⁶ independently of one another are hydrogen, (C₆-C₁₀)-aryl or 5- to 10-membered heteroaryl having up to three heteroatoms from the group consisting of N, O and S, where aryl and heteroaryl for their part may be substituted up to three times independently of one another by halogen, trifluoromethyl, trifluoromethoxy, cyano, nitro, hydroxyl, amino, (C₁-C₆)-alkyl or (C₁-C₆)-alkoxy,
      adamantyl, (C₁-C₈)-alkyl whose chain may be interrupted by one or two oxygen atoms and which may be substituted up to three times independently of one another by hydroxyl, phenyl, trifluoromethyl, (C₃-C₈)-cycloalkyl, (C₁-C₆)-alkoxy, mono- or di-(C₁-C₆)-alkylamino, 5- or 6-membered heterocyclyl having up to three heteroatoms from the group consisting of N, O and S or by 5- to 10-membered heteroaryl having up to three heteroatoms from the group consisting of N, O and S,
      (C₃-C₈)-cycloalkyl which may be substituted up to three times independently of one another by (C₁-C₄)-alkyl, hydroxyl or oxo, or
      5- or 6-membered heterocyclyl having up to two heteroatoms from the group consisting of N, O and S, where N is substituted by hydrogen or (C₁-C₄)-alkyl, or
   R⁵ and R⁶ together with the nitrogen atom to which they are attached form a 4- to 7-membered saturated heterocycle which may contain up to two further heteroatoms from the group consisting of N, O and S and which is optionally substituted by hydroxyl, oxo or (C₁-C₆)-alkyl, which for its part may be substituted by hydroxyl,
   R⁷ represents (C₆-C₁₀)-aryl or 5- to 10-membered heteroaryl having up to three heteroatoms from the group consisting of N, O and S, where aryl and heteroaryl for their part may be substituted up to three times independently of one another by halogen, trifluoromethyl, trifluoromethoxy, cyano, nitro, hydroxyl, amino, (C₁-C₆)-alkyl or (C₁-C₆)-alkoxy,
      adamantyl, (C₁-C₈)-alkyl whose chain may be interrupted by one or two oxygen atoms and which may be, substituted up to three times independently of one another by hydroxyl, phenyl, trifluoromethyl, (C₃-C₈)-cycloalkyl, (C₁-C₆)-alkoxy, mono- or di-(C₁-C₆)-alkylamino, 5- or 6-membered heterocyclyl having up to three heteroatoms from the group consisting of N, 9 and S or by 5- to 10-membered heteroaryl having up to three heteroatoms from the group consisting of N, O and S,
      (C₃-C₈)-cycloalkyl which may be substituted up to three times independently of one another by (C₁-C₄)-alkyl, hydroxyl or oxo or
      5- or 6-membered heterocyclyl having up to two heteroatoms from the group consisting of N, O and S, where N is substituted by hydrogen or (C₁-C₄)-alkyl,
R² represents (C₁-C₈)-alkyl whose chain may be interrupted by a sulphur atom or an oxygen atom or by an S(O) or SO₂ group, phenyl, benzyl or 5- or 6-membered heteroaryl having up to two heteroatoms from the group consisting of N, O and S, where phenyl, benzyl and heteroaryl for their part may be substituted up to three times independently of one another by halogen, trifluoromethyl, trifluoromethoxy, cyano, nitro, hydroxyl, amino, (C₁-C₆)-alkyl or (C₁-C₆)-alkoxy, and
R³ represents a group of the formula *CH₂—OH or *C(O)—NR⁸R⁹,
   in which
   * represents the point of attachment,
   R⁸ and R⁹ independently of one another represent hydrogen or (C₁-C₆)-alkyl, or
R² and R³ together with the CH group to which they are attached form a group of the formula 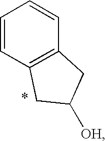
   in which
   * represents the point of attachment,
      and their salts, hydrates, hydrates of the salts and solvates.

Preference is furthermore given to compounds of the formula (I) according to the invention
in which
R¹ represents a group of the formula *C(═O)—R⁴ or *(CH2)ₐ—R⁴,
   in which
   * represents the point of attachment,
   a represents 1,
   R⁴ represents (C₆-C₁₀)-aryl or 5- to 10-membered heteroaryl having up to three heteroatoms from the group consisting of N, O and S, where aryl or heteroaryl for their part may be: substituted up to three times independently of one another by halogen, trifluoromethyl, trifluoromethoxy, cyano, nitro, hydroxyl, amino, (C₁-C₆)-alkyl or (C₁-C₆)-alkoxy,
R² represents (C₁-C₆)-alkyl whose chain may be interrupted by a sulphur atom or an oxygen atom or by an S(O) or SO₂ group, phenyl, benzyl or 5- or 6-membered heteroaryl having up to two heteroatoms from the group consisting of N, O and S, where phenyl, benzyl and heteroaryl for their part, may be substituted up to three times independently of one another by halogen, hydroxyl, amino, (C₁-C₄)-alkyl or (C₁-C₄)-alkoxy, and
R³ represents a group of the formula *C(O)—NR⁸R⁹,
   in which
   * represents the point of attachment,
   R⁸ and R⁹ independently of one another represent hydrogen, methyl or ethyl, or
R² and R³ together with the CH group to which they are attached form a group of the formula 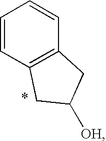
   in which
   * represents the point of attachment,
      and their salts, hydrates, hydrates of the salts and solvates.

Particular preference is given to compounds of the formula (I) according to the invention
in which
R¹ represents a group of the formula *C(═O)—R⁴,
   in which
   * represents the point of attachment,
   R⁴ represents (C₆-C₁₀)-aryl or 5- to 10-membered heteroaryl having up to three heteroatoms from the group consisting of N, O and S, where aryl and heteroaryl for their part may be substituted up to three times independently of one another by halogen, trifluoromethyl, trifluoromethoxy, cyano, nitro, hydroxyl, amino, (C₁-C₆)-alkyl or (C₁-C₆)-alkoxy,
R² represents phenyl which may optionally be substituted by fluorine in the para position to the point of attachment, or pyridyl, and
R³ represents a group of the formula *C(O)—NR⁸R⁹,
   in which
   * represents the point of attachment,
   R⁸ and R⁹ represent hydrogen,
      and their salts, hydrates, hydrates of the salts and solvates.

Moreover, we have found a process for preparing the compounds of the formula (I) according to the invention where either
[A] compounds of the formula (II) 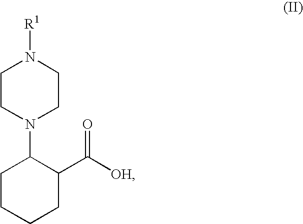
   in which
R¹ is as defined above,
are reacted with compounds of the formula (III) 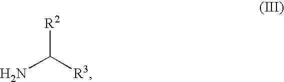
   in which
R² and R³ are as defined above, or
[B] compounds of the formula (IV) 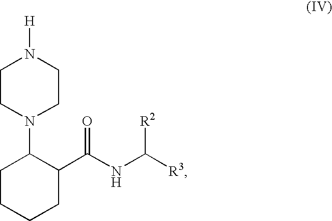
   in which
R² and R³ are as defined above
are reacted with compounds of the formula (V), (Va) or (Vb)
   R¹—X (V),
   R⁵R⁶ N═C═O (Va)
   R⁴—(CH₂)_{a—1}—CHO (Vb),
   in which
R¹, R⁵, R⁶ are as defined above,
a represents 1, 2 or 3 and
X represents a suitable leaving group, such as, for example, halogen, mesylate or tosylate, or represents a hydroxyl group.

The compounds of the formula (I) obtained according to process variant [A] or [B] can, if appropriate, subsequently be converted into the corresponding salts, for example by reaction with an acid.

The process according to the invention can be illustrated in an exemplary manner by the formula scheme below:

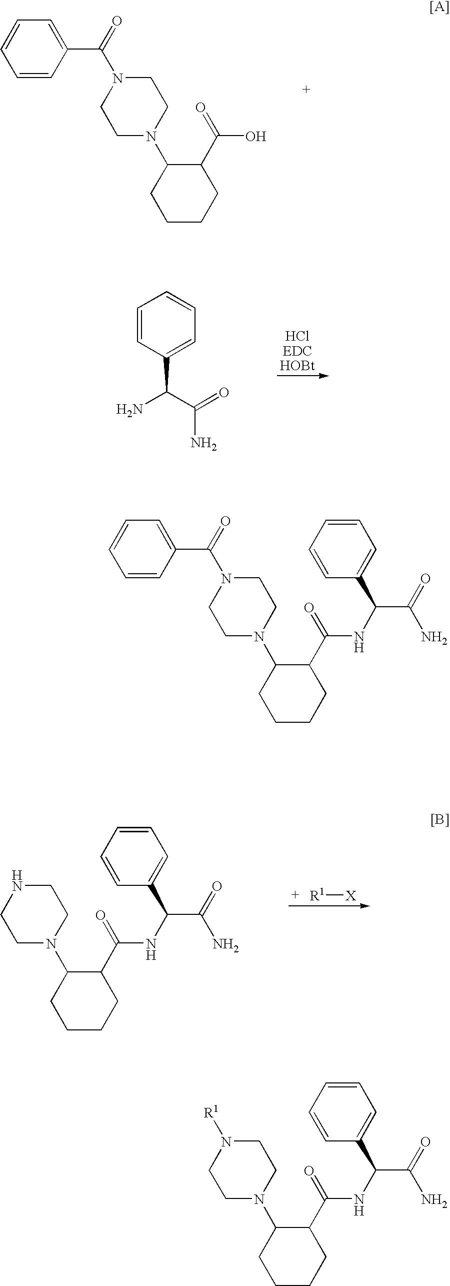

Compounds of the formula (II) can be prepared, for example, by converting X compounds of the formula (VI) 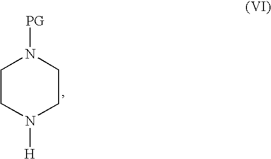
in which
PG represents an amino protective group
with compounds of the formula (VII) 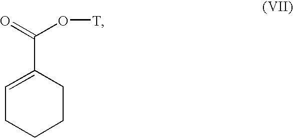
   in which
T represents (C₁-C₈)-alkyl, preferably tert-butyl,
if appropriate in the presesnce of a base, into compounds of the formula (VIU) 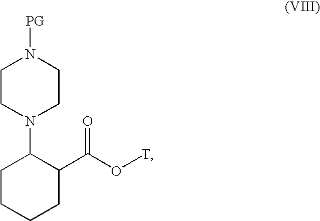
   in which
PG and T are as defined above,
which are then, by removal of the amino protective group, converted into compounds of the formula (IX) 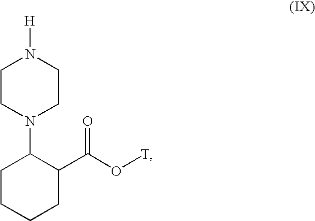
   in which
T is as defined above,
which are then converted, using compounds of the formula (V), (Va) or (Vb)
   R¹—X (V)
   R⁵R⁶ N═C═O (Va),
   R⁴—(CH₂)_{a—1}—CHO (Vb),
   in which
R¹, R⁵, R⁶ are as defined above,
a represents 1, 2 or 3 and
X represents a suitable leaving group, such as, for example, halogen, mesylate or tosylate, or represents a hydroxyl group,
into compounds of the formula (X) 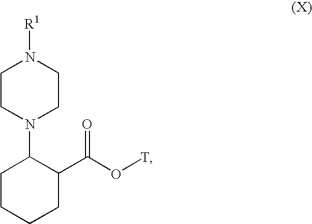
   in which
R¹ and T are as defined above,
giving finally, by cleavage of the ester group, the corresponding carboxylic, acids of the formula (I).

The following scheme illustrates this reaction sequence for preparing compounds of the formula (Il):

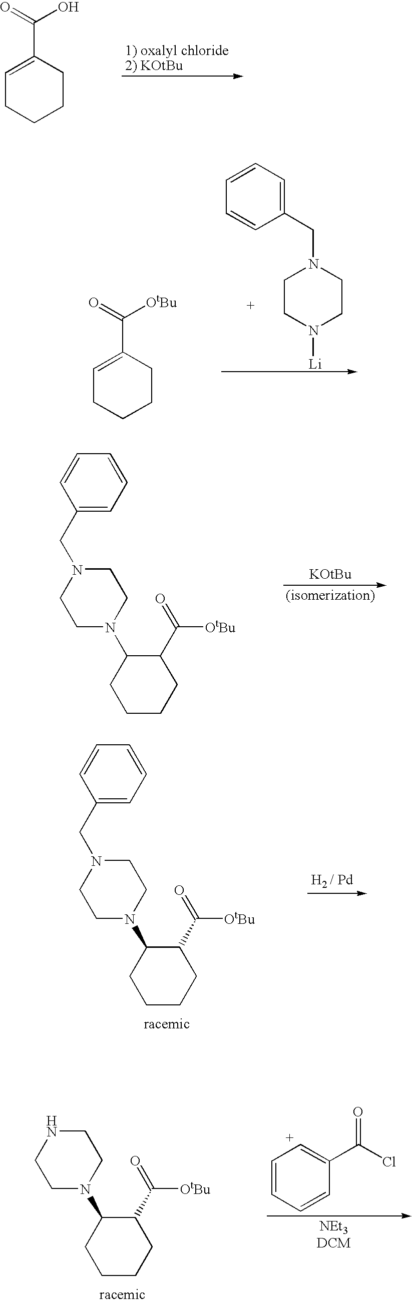

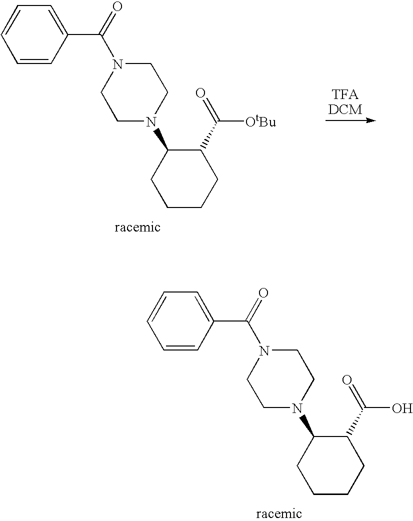

Compounds of the formula (X) in which R¹ represents a group of the formula
*SO₂—R⁴,
in which
* and R⁴ are as defined above
can also be prepared by reacting compounds of the formula (XI) 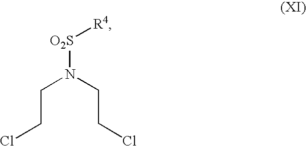
   in which
R⁴ is as defined above
with compounds of the formula (XII) 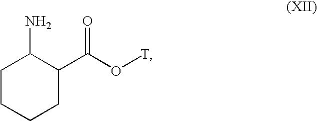
   in which
T is as defined above.

The following scheme illustrates this specific reaction sequence for preparing compounds of the formula (X):

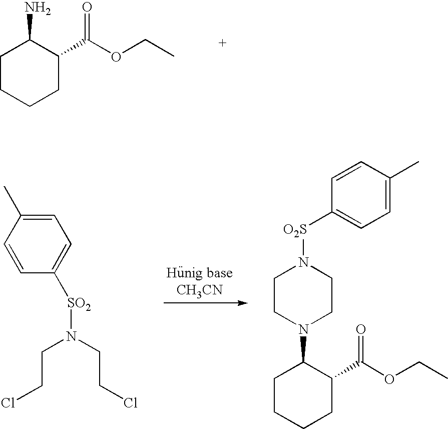

Compounds of the formula (IV) can be prepared, for example, by converting compounds of the formula (VIII) by cleavage of the ester group into compounds of the formula (XIII) 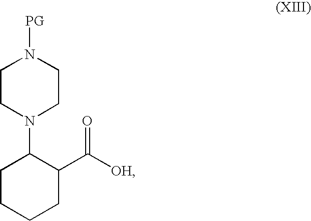
in which
PG is as defined above,
which are then reacted with compounds of the formula (I) to give compounds of the formula (XIV) 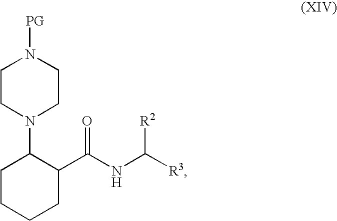
   giving finally, by removal of the amino protective group, the corresponding amines of the formula (IV).

The following scheme illustrates this reaction sequence for preparing compounds of the formula (IV):

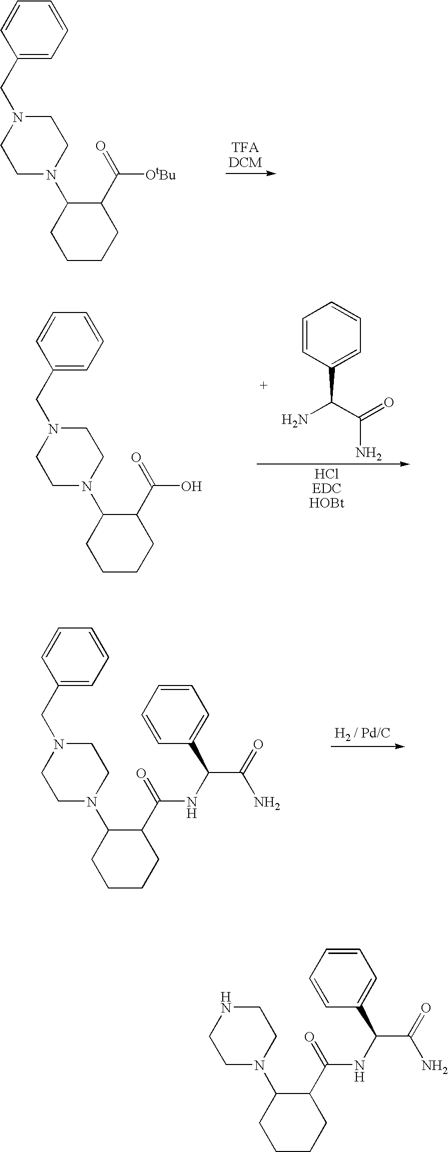

The compounds of the respective diastereomeric and enantiomeric forms are prepared correspondingly, either using enantiomerically or diastereomerically pure starting materials, by subsequent separation of the racemates formed using customary methods (for example racemate resolution, chromatography on chiral columns, etc.) or else by isomerization in the presence of a base, for example in order to convert the two substituents on the cyclohexyl ring into the trans configuration, preferably at the stage of the compounds of the formula (VIII).

The processes described above are generally carried out under atmospheric pressure. However, it is also possible to carry out the processes under elevated pressure or under reduced pressure (for example in the range from 0.5 to 5 bar).

In the context of the invention, customary amino protective groups are the amino protective groups used in peptide chemistry.

These preferably include: benzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyl-oxycarbonyl, 4-nitrobenzyloxycarbonyl, 2-nitro-4,5-dimethoxybenzyloxycarbonyl, methoxycarbonyl, ethoxycarbonyl, propoxycarbonyl, isopropoxycarbonyl, butoxy-carbonyl, isobutoxycarbonyl, tert-butoxycarbonyl, allyloxycarbonyl, vinyloxy-carbonyl, 2-nitrobenzyloxycarbonyl, 3,4,5-trimethoxybenzyloxycarbonyl, cyclo-hexoxycarbonyl, 1,1-dimethylethoxycarbonyl, adamantylcarbonyl, phthaloyl, 2,2,2-trichlorethoxycarbonyl, 2,2,2-trichloro-tert-butoxycarbonyl, menthyloxycarbonyl, phenoxycarbonyl, 4-nitrophenoxycarbonyl, fluorenyl-9-methoxycarbonyl formyl, acetyl, propionyl, pivaloyl, 2-chloroacetyl, 2-bromoacetyl, 2,2,2-trifluoroacetyl, 2,2,2-trichloroacetyl, benzoyl, 4-chlorobenzoyl, 4-bromobenzoyl, 4-nitrobenzoyl, phthalimido, isovaleroyl or benzyloxymethylene, benzyl, methoxybenzyl, 4-nitrobenzyl, 2,4-dinitrobenzyl, trityl, diphenylmethyl or 4-nitrophenyl. Preferred protective groups for secondary amines are benzyloxycarbonyl and tert-butoxycarbonyl.

The amino protective groups are removed in a manner known per se, using, for example, hydrogenolytic, acidic or basic conditions, preferably acids, such as, for example, hydrochloric acid or trifluoroacetic acid, in inert solvents, such as ether, dioxane and methylene chloride.

Solvents suitable for the process are customary organic solvents which do not change under the reaction conditions. These include ethers, such as diethyl ether, dioxane, tetrahydrofuran, glycol dimethyl ether, or hydrocarbons, such as benzene, toluene, xylene, hexane, cyclohexane or mineral oil fractions, or halogenated hydrocarbons, such as dichloromethane, trichloromethane, carbon tetrachloride, dichloroethylene, trichloroethylene or chlorbenzene, or ethyl acetate, pyridine, dimethyl sulphoxide, dimethylformamide, N,N′-dimethylpropyleneurea (DMPU), N-methylpyrrolidone (NMP), acetonitrile, acetone or nitromethane. It is also possible to use mixtures of the solvents mentioned.

Bases suitable for the processes are, in general, inorganic or organic bases. These preferably include alkali metal hydroxides, such as, for example, sodium hydroxide or potassium hydroxide, alkaline earth metal hydroxides, such as, for example, barium hydroxide, alkali metal carbonates, such as sodium carbonate, potassium carbonate or caesium carbonate, alkaline earth metal carbonates, such as calcium carbonate, or alkali metal or alkaline earth metal alkoxides, such as sodium methoxide or potassium methoxide, sodium ethoxide or potassium ethoxide or potassium tert-butoxide, or organic amines, such as triethylamine, or heterocycles, such as 1,4-diazabicyclo[2.2.2]octane (DABCO), 1,8-diazabicyclo[5.4.0]undec-7-ene (DBU), 1,5-diazabicyclo[4.3.0]non-5-ene (DBN), pyridine, N,N-dimethylamino-pyridine, N-methylpiperidine or N-methylmorpholine. It is also possible to use alkali metals such as sodium or their hydrides, such as sodium hydride, as bases.

The amide formation in process step (II)+(III)→(I) and (XIII)+(III)→(XIV) is preferably carried out in the solvent dimethylformamide or dichloromethane, in a temperature range from 0° C. to +100° C.

Preferred auxiliaries used for the amide formation are customary condensing agents, such as carbodiimides, for example N,N′-diethyl-, N,N,′-dipropyl-, N,N′-diisopropyl-, N,N′-dicyclohexylcarbodiimide, N-(3-dimethylaminopropyl)-N′-ethyl-carbodiimide hydrochloride (EDC), or carbonyl compounds, such as carbonyldi-imidazole, or 1,2-oxazolium compounds, such as 2-ethyl-5-phenyl-1,2-oxazolium 3-sulphate or 2-tert-butyl-5-methyl-isoxazolium perchlorate, or acylamino compounds, such as 2-ethoxy-1-ethoxycarbonyl-1,2-dihydroquinoline, or propanephosphonic anhydride, or isobutyl chloroformate, or bis-(2-oxo-3-oxa-zolidinyl)-phosphoryl chloride or benzotriazolyloxy-tri(dimethylamino)phosphonium hexafluorophosphate, or O-(benzotriazol-1-yl)-N,N,N′,N′-tetra-methyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyl-uronium hexafluorophosphate (HATU), if appropriate in combination with further auxiliaries, such as 1-hydroxybenzotriazole or N-hydroxysuccinimide, and the bases used are preferably alkali metal carbonates, for example sodium carbonate or potassium carbonate, or sodium bicarbonate or potassium bicarbonate, or organic bases, such as trialkylamines, for example triethylamine, N-methylmorpholine, N-methylpiperidine or diisopropylethylamine. Particular preference is given to the combination of EDC, N-methylmorpholine and 1-hydroxybenzotriazole.

If X in the compounds of the formula (V) represents a leaving group, such as, for example, halogen, mesylate or tosylate, the process steps (IV)+(V)→(I) and (IX)+(V)→(X) are preferably carried out in the solvent dichloromethane, in particular in the presence of a base, preferably triethylamine or pyridine, in a temperature range of from 0° C. to +100° C., preferably at room temperature.

If X represents a hydroxyl group, the reaction is preferably carried out under the preferred reaction conditions described above for the amide formation in process step (II)+(III)→(I) and (XII)+(III)→(XIV).

Reactions with isocyanates (Va) are preferably carried out in the solvent toluene or methylene chloride at a temperature of from 0° C. to 120° C., in particular at from 0° C. to 70° C.

Reactions with aldehydes (Vb) are preferably carried out in the solvent methanol, dichloromethane or 1,2-dichloroethane in the presence of sodium borohydride or sodium triacetoxyborohydride at a temperature of from 0° C. to 80° C., in particular from 0° C. to 40° C.

The process step (VI)+(VII)→(VIII) is preferably carried out in the solvent tetrahydrofuran in the presence of a base, in particular the combination n-butyl-lithium/N,N′,N″,N′″-tetramethylethylenediamine (TMEDA), at a temperature between −78° C. and +25° C., in particular between −70° C. and −20° C.

The removal of the amino protective group in process step (VIII)→(IX) and (XIV)→(IV) is in each case carried out under standard conditions. In the case of a benzyl protective group, its removal is preferably carried out in the solvent ethanol by hydrogenation using 10% palladium on activated carbon as catalyst at atmospheric pressure.

The hydrolysis of the carboxylic esters in process step (X)→(II) and (VIII)→(XIII) is carried out by customary methods, preferably in a temperature range of from 0° C. to +100° C., by treating the esters in inert solvents with bases, where the salts that are initially formed are converted by treatment with acid into the free carboxylic acids. In the case of the t-butyl esters the hydrolysis is preferably carried out using acids.

Solvents suitable for the hydrolysis of the carboxylic esters are water or the organic solvents which are customary for ester hydrolysis. These preferably include alcohols, such as methanol, ethanol, propanol, isopropanol or butanol, or ethers, such as tetrahydrofuran or dioxane, dimethylformamide, dichlordmethane or dimethyl sulphoxide. It is also possible to use mixtures of the solvents mentioned. Preference is given to water/tetrahydrofuran and, in the case of the reaction with trifluoroacetic acid, dichloromethane and, in the case of hydrogen chloride, tetrahydrofuran, diethyl ether, dichloromethane or dioxane.

Suitable bases are the alkali metal hydroxides or alkaline earth metal hydroxides preferred for the hydrolysis, such as, for example, sodium hydroxide, lithium hydroxide, potassium hydroxide or barium hydroxide, or alkali metal carbonates, such as sodium carbonate or potassium carbonate or sodium bicarbonate. Particular preference is given to using sodium hydroxide or lithium hydroxide.

Suitable acids are, in general, trifluoroacetic acid, sulphuric acid, hydrogen chloride, hydrogen bromide and acetic acid, or mixtures thereof, if appropriate with addition of water. Preference is given to hydrogen chloride or trifluoroacetic acid in the case of the tert-butyl esters and to hydrochloric acid in the case of the methyl esters.

When carrying out the hydrolyses, the base or the acid is generally employed in an amount of from 1 to 200 mol, preferably from 1.5 to 40 mol, based on 1 mol of ester.

The process step (XI)+(XII)→(X) is preferably carried out in the solvent acetonitrile in the presence of a base, in particular N-ethyldiisopropylamine, at a temperature of from 0° C. to 150° C., in particular between 60° C. and 130° C.

Surprisingly, the compounds of the formula (I) have an unforeseeable useful pharmacological activity spectrum and are therefore suitable in particular for the prophylaxis and/or treatment of disorders.

The compounds of the formula (I), alone or in combination with one or more other active compounds, are suitable for the prophylaxis and/or treatment of various disorders such as, in particular, ischaemia-related peripheral and cardiovascular disorders, for the acute and chronic treatment of ischaemic disorders of the cardiovascular system, such as, for example, coronary heart disease, stable and unstable angina pectoris, of peripheral and arterial occlusive diseases, of thrombotic vascular occlusions, of myocardial infarction and of reperfusion damage.

Moreover, owing to their potential to increase angiogenesis, they are particularly suitable for a permanent therapy of all occlusive diseases.

In addition, the compounds of the formula (I) can be used in particular for the prophylaxis and/or treatment of cerebral ischaemia, stroke, reperfusion damage, brain trauma, oedema, spasms, epilepsy, respiratory arrest, cardiac arrest, Reye syndrome, cerebral thrombosis, embolism, tumours, haemorrhages, encephalomyelitis, hydroencephalitis, spinal injuries, post-operative brain damage, injuries of the retina or the optic nerve following glaucoma, ischaemia, hypoxia, oedema or trauma, and also in the treatment of schizophrenia, sleep disturbances and acute and/or chronic pain and also neurodegenerative disorders, in particular for the treatment of cancer-induced pain and chronic neuropathic pain, such as, for example, in cases of diabetic neuropathy, post-therapeutic neuralgia, peripheral nerve damage, central pain (for example as a result of cerebral ischaemia) and trigeminal neuralgia and other chronic pain, such as, for example, lumbago, lower back pain or rheumatic pains.

The compounds of the formula (I) can furthermore also be used for treating hypertension and cardiac insufficiency, myocarditis, nephritis, pancreatitis, diabetic nephropathy, oedema and for potentiating the effect of nucleobase, nucleoside or nucleotide antimetabolites in cancer chemotherapy and antivirals (for example HIV) chemotherapy.

The present invention also relates to the use of the compounds of the formula (I) for preparing pharmaceuticals for the prophylaxis and/or treatment of the abovementioned syndromes.

The present invention furthermore relates to a method for the prophylaxis and/or treatment of the abovementioned syndromes using the compounds of the formula (I).

The pharmaceutical activity of the compounds of the formula (I) can be explained by their action as adenosine-uptake inhibitors.

The present invention furthermore provides pharmaceuticals comprising at least one compound of the formula (I), preferably together with one or more pharmaceutically acceptable auxiliaries or carriers, and their use for the abovementioned purposes.

Suitable for administering the compounds of the formula (I) are all customary administration forms, i.e. oral, parenteral, inhalative, nasal, sublingual, rectal, local such as, for example, in the case of implants or stents, or external, such as, for example, transdermal. In the case of parenteral administration, particular mention may be made of intravenous, intramuscular or subcutaneous administration, for example as a subcutaneous depot. Preference is given to oral or parenteral administration.

To this end, the active compounds can be administered alone or in the form of formulations. Formulations suitable for oral administration are inter alia tablets, capsules, pellets, sugar-coated tablets, pills, granules, solid and liquid aerosols, syrups, emulsions, suspensions and solutions. Here, the active compound has to be present in such an amount that a therapeutic effect is achieved. In general, the active compound can be present in a concentration of from 0.1 to 100% by weight, in particular from 0.5 to 90% by weight, preferably from 5 to 80% by weight. The concentration of the active compound should be in particular 0.5-90% by weight, i.e. the active compound should be present in amounts sufficient to achieve the stated dosage range.

To this end, the active compounds can be converted in a manner known per se into the customary formulations. This is achieved by using inert non-toxic pharmaceutically suitable carriers, auxiliaries, solvents, vehicles, emulsifiers and/or dispersants.

Auxiliaries which may be mentioned are, for example: water, non-toxic organic solvents, such as, for example, paraffins, vegetable oils (for example sesame oil), alcohols (for example ethanol, glycerol), glycols (for example polyethylene glycol), solid carriers, such as ground natural or synthetic minerals (for example talc or silicates), sugar (for example lactose), emulsifiers, dispersants (for example polyvinylpyrrolidone) and lubricants (for example magnesium sulphate).

In the case of oral administration, the tablets may, of course, also comprise additives, such as sodium citrate, together with fillers, such as starch, gelatin and the like. Aqueous formulations for oral administration may furthermore contain flavour enhancers or colorants.

In the case of parenteral administration, it has generally been found to be advantageous to administer amounts from about 0.0001 to about 10 mg/kg, preferably from about 0.003 to about 1 mg/kg, of body weight to obtain effective results. In the case of oral administration, the amount is from about 0.1 to about 20 mg/kg, preferably from about 0.3 to about 10 mg/kg, of body weight.

In spite of this, it may be required, if appropriate, to deviate from the amounts mentioned, namely depending on body weight, the route of administration, the individual response to the active compound, the type of formulation and the time or interval at which administration takes place.

The present invention is illustrated by the non-limiting preferred examples below.

In the examples below, percentages are, unless indicated otherwise, always based on weight; parts are parts by weight.

A Assessment of the Physiological Activity

1. Inhibition of the Adenosine Uptake in Rabbit Erythrocytes by the Compounds According to the Invention

The capability of substnaces to influence the adenosine-uptake system is investigated by determining the inhibitory effect of the substances on functional adenosine uptake.

For the functional adenosine-uptake test, an erythrocyte preparation from rabbit blood is used. The blood is drawn intravenously using citrate (3 ml Monovette 9NC from Sarstedt) as anticoagulant. The blood is centrifuged at 3000 g for 5 min and the erythrocytes are suspended in 10 mM 3-(N-morpholino)propanesulphonic acid buffer (MOPS)/0.9% aqueous sodium chloride solution pH 7.4. The suspension is diluted to one hundredth of the original blood volume. In each case, 990 μl of the suspension are admixed with 10 μl of a suitable concentration of the substance to be examined, and the mixture is incubated at 30° C. for 5 min. 5 μl of a 4 mM adenosine solution are then added, and the mixture is incubated at 30° C. for another 15 min. The samples are then centrifuged at 3000 g for 5 min and in each case 700 μl of the supernatant are mixed with 28 μl of 70% strength HClO₄, allowed to stand in an ice bath for 30 min and centrifuged at 16 000 g for 3 min, and 350 μl of the sample are neutralized using 30 μl of 5 N NaOH. 50 μl of the sample are applied to a column (Waters Symmetry C18 5 μm, 3.9×150 mm). A Spherisorb ODS II 5 μm, 4.6×10 mm column is used as precolumn The mobile phase used is a gradient of 50 mM KH₂PO₄/5 mM tributylamine pH 7 (mobile phase A) and a mixture of mobile phase A/methanol 1:1 (mobile phase B). The gradient is from 10% to 40% B, at a flow rate of 0.5 ml/min. The adenosine which is present is quantified by its absorption at 260 nm, as are the hypoxanthine and inosine formed. The IC₅₀ is the concentration of active compound at which, 15 min after addition of adenosine, 50% of the adenosine concentration originally employed is still present.

Table 1 below lists the IC₅₀ values obtained using this test:

TABLE 1

| Example No. | IC₅₀ [nM] |
|---|---|
| 1 | 30 |
| 2 | 30 |
| 3 | 30 |
| 8 | 80 |
| 16 | 30 |
| 17 | 20 |
| 18 | 30 |
| 21 | 15 |
| 27 | 20 |
| 66 | 20 |

2. In vivo Test Model for Testing Adenosine-Uptake Inhibitors

Adult mongrel dogs (body weight 20-30 kg) are initially anaesthetized using a combination of trapanal 500 mg and alloferin 55 mg. Anaesthesia is maintained by infusion of a mixture of fentanyl 0.072 mg/kg, alloferin 0.02 mg/kg and dihydrobenzpyridyl 0.25 mg/kg×min. The animals are intubated and ventilated with a mixture of O₂/N₂O (ratio 1:5) using a Dräger ventilation pump at 16 breaths per min and a volume of 18-24 ml/kg. The body temperature is maintained at 38° C.±0.1° C. Arterial blood pressure is measured via a catheter in the femoral artery. A thoractomy is carried out on the left side at the fifth intercostal space. The lung is pushed back and fixed and a cut is made in the pericardium. A proximal section of the LAD distally to the first diagonal branch is exposed and a calibrated electromagnetic flow sensor (from Scalar) is placed around the vessel and attached to a flow meter (from Scalar, model MDL 1401). Distally to the flow sensor, a mechanical occluder is attached such that there are no branches in between flow sensor and occluder.

Using a catheter in the femoral vein, blood samples are taken and substances (10 μg/kg i.v.) are administered. A peripheral ECG is recorded using needles which are fixed subcutaneously. A microtip pressure manometer (from Millar, model PC-350) is pushed through the left atrium to measure the pressure in the left ventricle. Measurement of the heart frequency is triggered by the R wave of the ECG. During the entire experiment, the haemodynamic parameters and the coronary flow are recorded using a multi-event recorder.

A four-minute occlusion causes reactive hyperaemia. The difference between the coronary flow under control conditions and the maximum flow during the reactive hyperaemia is measured. The time which is required to achieve half of this maximum flow in the drop is a suitable parameter to assess the reaction hyperaemia.

After a stabilization period of one hour, the experiment is started with a four-minute occlusion. Thirty minutes later, the substance is administered (i.v.) which is, after two minutes, followed by re-occlusion. The reactive hyperaemia after verum and placebo is compared.

Table 2 below lists the activity data obtained in this model:

TABLE 2

| Example No. | Increase of coronary blood flow in % |
|---|---|
| 1 | 258 |
| 16 | 326 |
| 17 | 250 |
| 18 | 347 |

### B PREPARATION EXAMPLES

Abbreviations:

| | |
|---|---|
| abs. | absolute |
| DCI | direct chemical ionization (in MS) |
| DMAP | 4-N,N-dimethylaminopyridine |
| DMF | N,N-dimethylformamide |
| DMSO | dimethyl sulphoxide |
| EDC | N′-(3-dimethylaminopropyl)-N-ethylcarbodiimide x HCl |
| ESI | electrospray ionization (in MS) |
| GC | gas chromatography |
| HOBt | 1-hydroxyl-1H-benzotriazole x H₂O |
| HPLC | high pressure, high performance liquid chromatography |
| b.p. | boiling point |
| MS | mass spectroscopy |
| R_{f} | retention index (in TLC) |
| RT | room temperature |
| Rₜ | retention time (in HPLC) |
| THF | tetrahydrofuran |
| TMEDA | N,N,N′N′-tetramethylethylenediamine |

### Example 1

(1R,2R)-N-[(1S)-2-Amino-2-oxo-1-phenylethyl]-2-(4-benzoyl-1-piperazinyl)cyclo-hexanecarboxamide 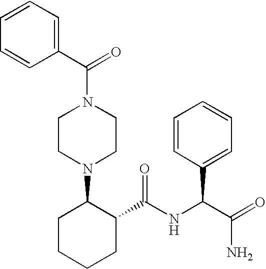
Step 1a):
tert-Butyl 1-cyclohexenecarboxylate

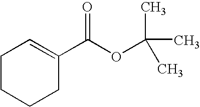

At 0° C., 98.7 g (0.78 mol) of 1-cyclohexenecarboxylic acid are initially charged in dichloromethane, and 81.9 ml (0.94 mol) of oxalyl chloride are added with stirring such that the temperature does not exceed 3° C. After the addition has ended, the reaction mixture is stirred at RT for 3 h. Evolution of gas can be observed. The reaction solution is concentrated, toluene (350 ml) is added and the mixture is re-concentrated to dryness. The residue is taken up in abs. THF (700 ml) and cooled to 10° C., and a solution of 105.3 g (0.94 mol) of potassium tert-butoxide in abs. THF (350 ml) is added such that the temperature does not exceed 15-20° C. The reaction mixture is stirred overnight, added to water (0.71) and extracted three times with in each case 500 ml of diethyl ether, and the combined organic phases are washed with saturated sodium chloride solution, dried over sodium sulphate, filtered and concentrated to dryness. This gives 142.3 g of crude producct which is purified on 3 kg of silica gel (0.06 to 0.2 mm) using the mobile phase petroleum ether/dichloromethane 1:1. 106.5 g of product are isolated and, for further purification, distilled under reduced pressure. This gives 92.0 g (65% of theory) of the desired ester.
b.p. (3.4 mbar): 67° C.

R_{f} (dichloromethane)=0.67

HPLC (method A): Rₜ=5.08 min.

MS (GC-MS; CI): m/z=183 (M+H)⁺, 200 (M+NH₄)⁺

¹H-NMR (300 MHz, CDCl₃): δ=1.48 (s, 9H), 1.53-1.70 (m, 4H), 2.12-2.25 (m, 4H), 6.87 (m, 1H).

Step 1b):

rac-cis/trans-tert-Butyl 2-(4-benzyl-1-piperazinyl)cyclohexanecarboxylate

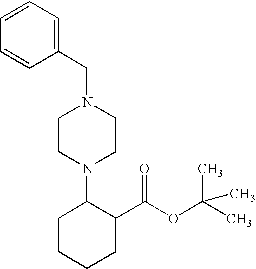

Two identical reactions are carried out:

93.2 ml (0.54 mol) of N-benzylpiperazine and 80.9 ml (0.54 mol) of TMEDA are dissolved in 800 ml of abs. THF. At 0° C., 214 ml (0.54 mol) of 2.5 N n-butyllithium solution in hexane are added and the mixture is stirred at 0° C. for 25 min. The reaction mixture is cooled to −66° C. and a solution of 81.4 g (0.45 mmol) of the ester from step 1a) in 480 ml of THF is added dropwise. The reaction solution is stirred at the same temperature for 1 h and allowed to stand at −26° C. overnight. The reaction is terminated by addition of a solution of 74 ml of methanol in 136 ml of THF and 10 min of stirring at RT.

Both batches are combined and concentrated using a rotary evaporator. The resulting oil is extracted with dichloromethane (4l) and water (0.7l), the phases are separated and the aqueous phase is extracted twice with in each case 500 ml of dichloromethane. The combined organic phases are washed with 500 ml of saturated sodium chloride solution, dried over sodium sulphate and filtered, and the solvent is removed under reduced pressure. The residue (about 400 g) is purified on 8 kg of silica gel (0.06-0.2 mm) using the mobile phase methanol/dichloromethane 1:9. This gives 260 g of product fraction which contains mainly the cis product, furthermore the trans product and additionally a by-product. This product is used without further purification for the next step.

cis-Product:

R_{f} (methanol/dichloromethane 1:10)=0.44

HPLC (method A): Rₜ=3.92 min.

MS (DCI/NH₃): m/z=359 (M+H)⁺

Step 1c):

(1R*,2R*)-tert-Butyl 2-(4-benzyl-1 piperazinyl)cyclohexancarboxylate 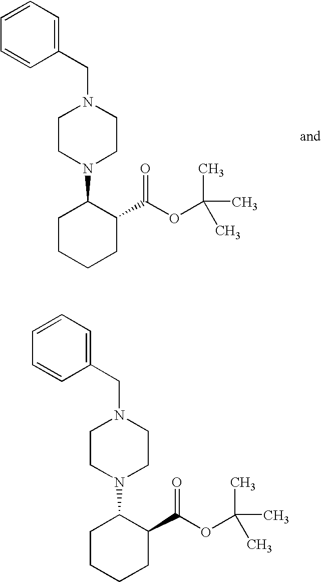
Method A:

Two identical reactions are carried out:

The compound from step 1b) (130 g) and 222 g (1.81 mol) of potassium tert-butoxide are dissolved in THF (2.86 l), and tert-butanol (173 ml) is added. The reaction mixture is stirreed at RT for 5 days, and both batches are combined for work-up. The reaction solution is diluted with 11 l of dichloromethane and washed four times with in each case 2 l of water. The combined aqueous phases are extracted twice with in each case 2 l of dichloromethane and the combined organic phases are washed with saturated sodium chloride solution and dried over sodium sulphate. After filtration, the filtrate is concentrated and the residue is purified chromatographically on 8 kg of silica gel (0.063-0.2 mm) using the mobile phases cyclohexane/ethyl acetate 7:3. This gives 98.3 g (31% of theory) of the racemic trans product.

R_{f} (methanol/dichloromethane 1:10)=0.54

HPLC (method A): Rₜ=4.23 min.

MS (DCI/NH₃): m/z=359 (M+H)⁺

¹H-NMR (300 MHz, CDCl₃): δ=1.01-1.32 (m, 3H), 1.44 (s, 9H), 1.40-1.52 (m, 1H), 1.62-1.72 (m, 1H), 1.73-1.91 (m, 3H), 2.28 (dt, 1H, Jₜ=11.5 Hz, J_{d}=3.6 Hz), 2.32-2.48 (m, 6H), 2.58 (dt, 1H, Jₜ=11.2 Hz, J_{d}=3.1 Hz), 2.68-2.79 (m, 2H), 3.47 (t, 2H, J=13.2 Hz), 7.19-7.33 (m, 5H).

Method B:

215 g (1.22 mol) of N-benzylpiperazine and. 142 g (1.22 mol) of TMEDA are dissolved in 1.85 l of abs. TBF, and at 0° C., 487 ml (1.22 mol) of a 2.5 N n-butyllithium solution in hexane are added, and the mixture is stirred at 0° C. for 25 min. The reaction mixture is cooled to −50° C. and a solution of. 185 g (1.02 mol) of the ester from step 1a) in 1.11 l of THF is added dropwise. The reaction solution is stirred at the same temperature for 7 h and the reaction is terminated at this temperature by addition of methanol (200 ml). The temperature increases to −20° C. The mixture is stirred at RT for 10 min. The solvent is removed under reduced pressure, and the residue is taken up in ethyl acetate (1.85 l) and extracted with water (3.0 l). The aqueous phase is extracted once with ethyl acetate (925 ml) and the combined organic phases are washed with saturated sodium chloride solution (1.0 l). The organic phase is dried over sodium sulphate and filtered and the solvent is removed under reduced pressure. Without work-up, the residue (315 g) is, together with the 376 g (3.08 mol) of potassium tert-butoxide, taken up in TEF (3.94 l). At RT, 294 ml (3.08 mol) of tert-butanol are added, and the reaction mixture is stirred overnight. Water (24 l) is added and the mixture is extracted twice with in each case 4.0 l of ethyl acetate. The combined organic phases are washed with saturated sodium chloride solution (2.4 l), dried over sodium sulphate and filtered, and the solvent is removed using a rotary evaporator. Using dichloromethane, the residue is adsorbed on silica gel and purified by column chromatography on 4 kg of silica gel (0.063-0.20 mm) using the mobile phase cyclohexane/ethyl acetate 7:3. This gives 122 g (34% of theory) of the racemic trans product.

Step 1d):

(1R*,2R*)-tert-Butyl 2-(1-piperazinyl)cyclohexanecarboxylate

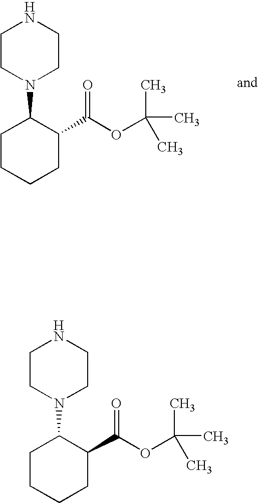

45.5 g (130 mmol) of the compound from step 1c) are, under argon, initially charged in ethanol (1.63 l), 9.78 g of 10% palladium on activated carbon are added and the mixture is then hydrogenated at RT and atmospheric pressure. After 2 h, the reaction mixture is filtered off with suction, through kieselguhr, the filter pad is washed with ethanol and the filtrate is concentrated and dried under high vacuum. Thi's gives 34 g (98% of theory) of product.

R_{f} (methanol/dichloromethane 1:10)=0.05

HPLC (method A): Rₜ=3.59 min.

MS ESI pos): m/z=269 (M+H)⁺

¹H-NMR (300 MHz, CDCl₃): δ=1.02-1.32 (m, 3H), 1.46 (s, 9H), 1.41-1.73 (m, 3H), 1.74-1.92 (m, 3H), 2.25-2.43 (m, 3H), 2.55 (dt, 1H, Jₜ=11.2 Hz, J_{d}=3.0 Hz), 2.64-2.74 (m, 2H), 2.81 (m, 4H).

Step 1e):

(1R*,2R*)-tert-Butyl 2-(4-benzoyl-1-piperazinyl)cyclohexanecarboxylate

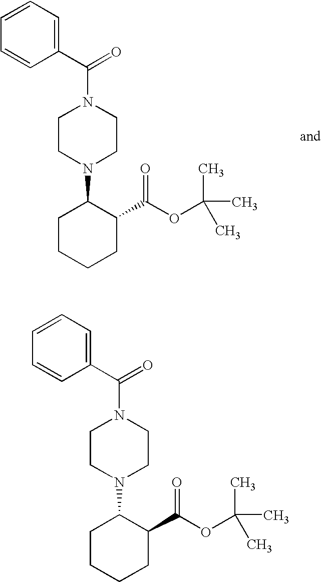

34 g (127 mmol) of the compound from step 1d) and 21.2 ml (152 mmol) of triethylamine are initially charged in dichloromethane (700 ml), and a solution of 14.7 ml (127 mmol) of benzoyl chloride is added dropwise at RT. The reaction mixture is stirred overnight at RT. The reaction mixture is washed twice in in each case 300 ml of water and the organic phase is dried over sodium sulphate, filtered, admixed with 250 g of silica gel (0.063-0.2 mm) and concentrated to dryness. The crude substance adsorbed on silica gel is purified by chromatography on 2 kg of silica gel (0.063-0.2 mm) using the mobile phase cyclohexane/ethyl acetate 7:3. This gives 42 g (89% of theory) of the product.

R_{f} (methanol/dichloromethane 1:10)=0.69

HPLC (method A): Rₜ=4.09 min.

MS (ESI pos): m/z=373 (M+H)⁺, 395 (M+Na)⁺

¹H-NMR (300 MHz, CDCl₃): δ=1.02-1.33 (m, 3H), 1.39-1.54 (m, 1H), 1.46 (s, 9H), 1.63-1.74 (m, 1H), 1.75-1.94 (m, 3H), 2.24-2.57 (m, 2H), 2.30 (dt, 1H, Jₜ=11.5 Hz, J_{d}=3.6 Hz), 2.58-2.89 (m, 2H), 2.65 (dt, 1H, Jₜ=11.3 Hz, J_{d}=3.0 Hz), 3.20-3.85 (m, 4H), 7.39 (s, 5H).

Step 1f):

1-Benzoyl-4-[(1R*,2R*)-2-carboxycyclohexyl]piperazin-4-ium trifluoroacetate

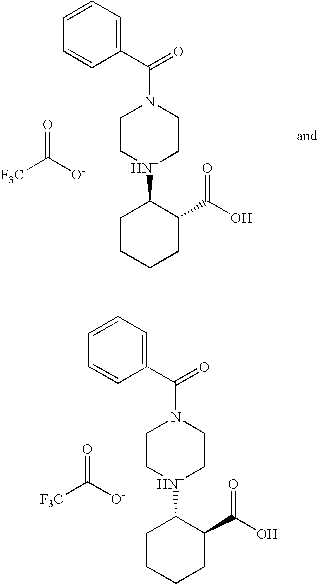

41.6 g (112 mmol) of the compound from step 1e) are dissolved in dichloromethane (705 ml), and trifluoroacetic acid (356 ml) is added at RT. The reaction mixture is stirred at RT overnight, concentrated and admixed five times with dichloromethane and twice with toluene and in each case reconcentrated. Using a bent tube, the remaining trifluoroacetic acid is distilled off at a bath temperature of 60° C. under high vacuum into a flask filled with liquid nitrogen. This gives 64.8 g of product which is reacted further without further purification.

R_{f} (methanol/dichloromethane 1:10)=0.21

HPLC (method A): Rₜ=3.38 min.

MS (ESI pos): m/z=317 (M+H)⁺

¹H-NMR (400 MHz, DMSO-d₆): δ=1.25 (m, 2H), 1.44 (m, 2H), 1.63 (br. d, 1H), 1.78 (br. d, 1H), 2.05 (br. d, 2H), 2.70 (br. dt, 1H), 3.03-3.45 (m, 4-5H), 3.62 (br. s, 2H), 4.5-6.5 (br. m, 34H), 7.43-7.53 (m, 5H).

Step 1g):

Diastereomer mixture of
(1R,2R)-N-[(1S)-2-amino-2-oxo-1-phenylethyl]-2-(4-benzoyl-1-piperazinyl)cyclo-hexanecarboxamide and (1S,2S)-N-[(1S)-2-amino-2-oxo-1-phenylethyl]-2-(4-benzoyl-1-piperazinyl)cyclohexanecarboxamide

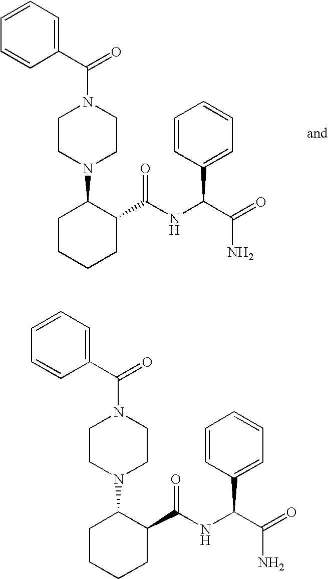

65 g (about 112 mmol) of the carboxylic acid from step 1f), 16.8 g (125 mmol) of HOBt and 25.0 g (130 mmol) of EDC are initially charged in DMF (1.03 l), 21.1 g (113 mmol) of (S)-phenylglycinamide hydrochloride, 74.7 ml (680 mmol) of N-methylmorpholine and a spatulatip of DMAP are added at RT and the reaction mixture is stirred at RT overnight. Water is added to the reaction solution, which is then extracted three times with ethyl acetate. The combined organic phases are washed with saturated sodium bicarbonate solution, dried over sodium sulphate and filtered, and the solvent is removed under reduced pressure. 6 h of drying under high vacuum give 48.3 g (95% of theory) of crude product which is directly separated into the two diastereomers using preparative HPLC.

Step 1h) (Separation of diastereomers):

(1R,2R)-N-[(1S)-2-Amino-2-oxo-1-phenylethyl]-2-(4-benzoyl-1-piperazinyl)cyclo hexanecarboxamide(diastereomer 1A) 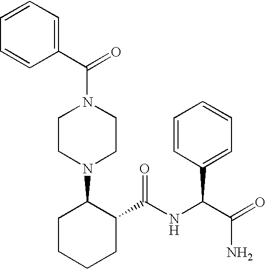
and
(1S,2S)-N-[(1S)-2-amino-2-oxo-1-phenylethyl]-2-(4-benzoyl-1-piperazinyl)cyclohexanecarboxamide (diastereomer 1B)

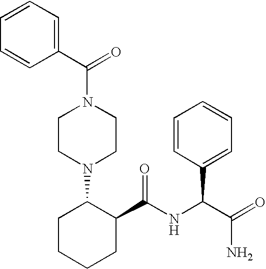

45.6 g of the mixture of diastereomers from step 1 g.) are dissolved in 250 ml of THF and, by preparative HPLC on Chromasil 100 C 18 (5 μm, 250×20 mm, 35° C., injection volume=0.33 ml, flow rate=25 ml/min) with acetonitrile/water 40:60 separated into the two diastereomers. This gives 16.0 g (35% of theory) of diastereomer A and 15.3 g (34% of theory) of diastereomer 1B.

Diastereomer 1A:

R_{f} (methanol/dichloromethane 1:10)=0.63

HPLC (method A): Rₜ=3.53 min.

MS (ESI pos): m/z=449 (M+H)⁺

¹H-NMR (400 MHz, CDCl₃): δ=1.06-1.22 (m, 3H), 1.22-1.36 (m, 1H), 1.68-1.92 (m, 3H), 2.20-2.29 (m, 2H), 2.30-2.57 (br. m, 2H), 2.58-2.85 (m, 3H), 3.35 (br. s, 2H), 3.71 (br. m, 2H), 5.52 (br. s, 1H), 5.60 (d, 1H), 6.04 (br. s, 1H), 7.29-7.44 (m, 10H), 9.35 (d, 1H).

Diastereomer 1B:

R_{f} (methanol/dichloromethane 1:10)=0.59

HPLC (method A): Rₜ=3.69 min.

MS (ESI pos): m/z=449 (M+H)⁺

¹H-NMR (200 MHz, CDCl₃): δ=0.98-1.47 (m, 4H), 1.60-1.97 (m, 3H), 2.12-2.33 (m, 2H), 2.33-2.90 (br. m, 5H), 3.15-3.70 (br. m, 3H), 3.72-3.98 (br. m, 1H), 5.54 (br. d, 2H), 6.22 (br. s, 1H), 7.29-7.46 (m, 10H), 9.47 (d, 1H).

### Example 2

(1R,2R)-N-[(1S)-2-Amino-2-oxo-1-(4-fluorophenyl)ethyl]-2-(4-benzoyl-1-piperazin-yl)cyclohexanecarboxamide (diastereomer 2A) 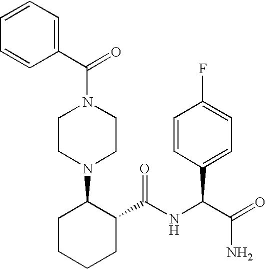
and
(1S,2S)-N-[(1S)-2-amino-2-oxo-1-(4-fluorophenyl)ethyl]-2-(4-benzoyl-1-piperazin-yl)cyclohexanecarboxanmide (diastereomer 2B)

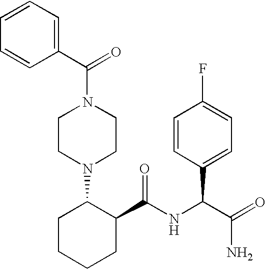

These compounds are prepared analogously to Example 1 by initially reacting 7.0 g (11.6 mmol, 71% purity) of the carboxylic acid from step 1f) analogously to step 1g) with 1.73 g (12.8 mmol) of HOBt, 2.56 g (13.3 mmol) of EDC and 2.38 g (11.6 mmol) of(S)-4-fluorophenylglycinamide hydrochloride, 7.7 ml (69.7 mmol) of N-methylmorpholine and a spatulatip of DMAP in DMP (105 ml); the resulting 2.84 g (49% of theory) of product (mixture of diastereomers) are then separated analogously to step 1h) by preparative HPLC into the two diastereomers. This gives 1.05 g each (38% of theory) of diastereomer 2A and diastereomer 2B.

Diastereomer 2A:

R_{f} (methanol/dichloromethane 1:10)=0.38

HPLC (method A): Rₜ=3.66 min.

MS (ESI pos): m/z=467 (M+H)⁺

¹H-NMR (300 MHz, CDCl₃): δ=1.04-1.36 (m, 4H), 1.67-1.96 (m, 3H), 2.18-2.31 (m, 2H), 2.31-2.57 (m, 2H), 2.57-2.91 (m, 3H), 3.20-3.95 (m, 4H), 5.43 (br. s, 1H), 5.55 (d, 1H), 5.88 (br. s, 1H), 7.04 (m, 2H), 7.35-7.43 (m, 7H), 9.43 (br. d, 1H).

Diastereomer 2B:

R_{f} (methanol dichloromethane 1:10)=0.38

HPLC (method A): Rₜ=3.82 min.

MS (ESI pos): m/z=467 (M+H)⁺

¹H-NMR (300 MHz, CDCl₃): δ=1.03-1.44 (m, 4H), 1.67-1.96 (m, 3H), 2.13-2.29 (m, 2H), 2.35-2.60 (m, 2H), 2.60-2.89 (m, 3H), 3.15-4.05, (m, 4H), 5.47 (br. s, 1H), 5.52 (d, 1H), 6.09 (br. s, 1H), 7.03 (m, 2H), 7.33-7.45 (m, 7H), 9.40 (br. d, 1H).

### Example 3

(1R,2R)-N-[(1S)-2-Amino-2-oxo-1-phenylethyl]-2-[4-(1H-indazol-3-ylcarbonyl)-1-piperazinyl]cyclohexanecarboxamide and

(1 S,2S)-N-[(1S)-2-amino-2-oxo-1-phenylethyl]-2-[4-(1H-indazol-3-ylcarbonyl)-1-piperazinyl]cyclohexanecarboxamide

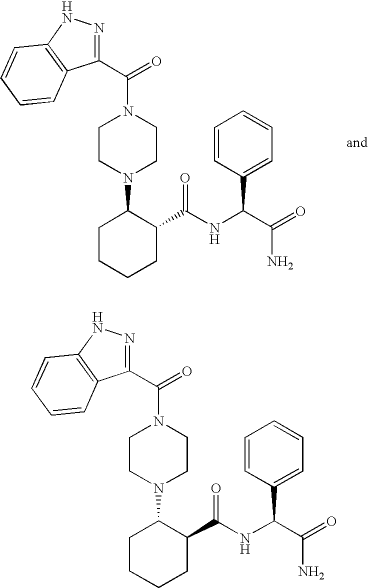

Step 3a):
1-(tert-Butoxycarbonyl)-1H-indazole-3-carboxylic acid

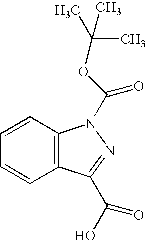

100 g (0.62 mol) of indazole-3-carboxylic acid and 163 g (1.54 mol) of sodium carbonate are initially charged in water (300 ml) and. THF (200 ml), and 148 g (0.68 mol) of di-tert-butyl pyrocarbonate are added at RT. The reaction mixture is stirred at RT overnight and then adjusted to pH 3 by addition of 5 N hydrochloric acid (evolution of gas). This solution is extracted with dichloromethane, the phases are separated and the aqueous phase is reextracted twice with dichloromethane. The combined organic phases are combined over sodium sulphate, filtered and concentrated. The residue is once more taken up in dichloromethane and reconcentrated to dryness. This gives 140 g (87% of theory) of product. The aqueous phase is concentrated to dryness and once more treated with water/dichloromethane as above, giving another 17.6 g (11%) of a product-containing fraction.

R_{f} (methanol/dichloromethane 1:5) 0.38

HPLC (method C): Rₜ=4.05 min.

MS (ESI pos): m/z=263 (M+H)⁺, 285 (M+Na)⁺

¹H-NMR (200 MHz, DMSO-d₆): δ=1.96 (s, 9H), 7.49 (t, 1H), 7.68 (t, 1H), 8.18 (m, 2H), 13.79 (br. s, 1H).

Step 3b):

tert-Butyl 3-({4-[(1R*,2R*)-2-(tert-butoxycarbonyl)cyclohexyl]-1-piperazinyl}-carbonyl)-1H-indazole-1-carboxylate

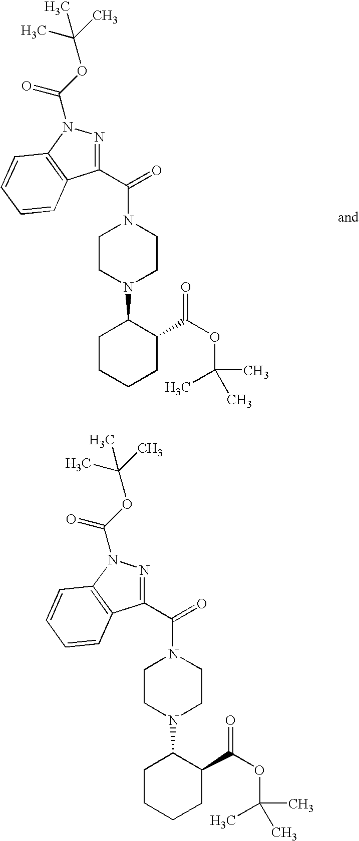

323 mg (1.23 mmol) of the carboxylic acid from step 3a), 183 mg (1.35 mmol) of HOBt and 271 mg (1.41 mmol) of EDC are initially charged in anhydrous DMF (10 ml). At RT, 330 mg (1.23 mmol) of the piperazine from Example 1/step 1d) and 0.41 ml (3.69 mmol) of N-methylmorpholine and a spatulatip of DMAP are added, and the reaction mixture is stirred at RT overnight. For work-up, the mixture is extracted with water and dichloromethane, the aqueous phase is extracted two more times with dichloromethane, the combined organic phases are dried over sodium sulphate and filtered and the solvent is removed under reduced pressure. The residue (768 mg) is purified chromatographically on silica gel using the mobile phase methanol/dichloromethane 1:20. This gives 482 mg (76% of theory) of the racemic product.

R_{f} (methanol dichloromethane 1:10)=0.72

HPLC (method C): Rₜ=4.26 min.

MS (ESI pos): m/z=513 (M+H)⁺

¹H-NMR (200 MHz, DMSO-d₆): δ=1.00-1.26 (m, 3H), 1.33-1.40 (m, 1H), 1.42 (s, 9H), 1.52-1.86 (m, 4H), 1.67 (s, 9H), 2.22-2.87 (m, 6H), 3.49-3.77 (m, 4H), 7.44 (t, 1H), 7.67 (t, 1H), 7.91 (d, 1H), 8.11 (d, 1H).

Step 3c):

3-({4-[(12R*)-2-Carboxycyclohexyl]piperazin-4-ium-1-yl}carbonyl)-1H-indazol-2-ium bis(trifluoroacetate)

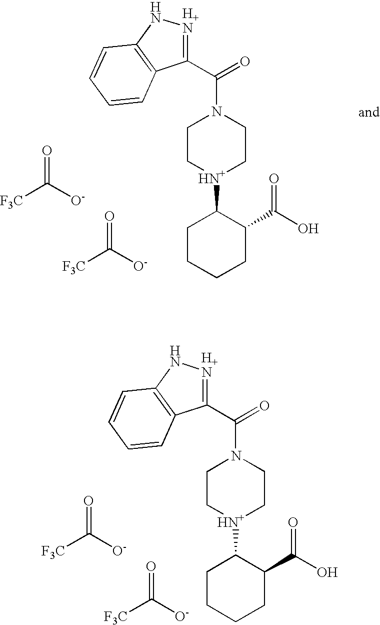

456 mg (0.89 mmol) of the tert-butyl ester from step 3b), are initially charged in dichloromethane (6 ml), and trifluoroacetic acid (3 ml) is added at RT. The reaction mixture is stirred at RT for 3.5 h and then evaporated to dryness, and the residue is taken up in dichloromethane, reconcentrated to dryness and dried under high vacuum. This gives 672 mg of a viscous oil product which is reacted further without further purification.

R_{f} (methanol/dichloromethane 1:10)=0.15

HPLC (method A): Rₜ=4.50 min.

MS (ESI pos): m/z 357 (M+H)⁺

¹H-NMR (300 MHz, DMSO-d₆): δ=1.13-1.35 (m, 2H), 1.35-1.56 (m, 3H), 1.56-1.67 (m, 1H), 1.67-1.84 (m, 1H), 1.96-2.14 (m, 2H), 2.77. (dt, 1H, Jₜ=11.0 Hz, J_{d}=3.8 Hz), 3.17-3.58 (m, 5H), 3.654.89 (m, 2H), 7.25 (t, 1H), 7.44 (t, 1H), 7.64 (d, 1H), 8.05 (d, 1H), 8.70-10.20 (m, 1H), 13.67 (s, 1H).

Step 3d):

Diastereomer mixture of

(1R,2R)-N-[(1S)-2-amino-2-oxo-1-phenylethyl]-2-[4-(1H-indazol-3-ylcarbonyl)-1-piperazinyl]cyclohexanecarboxamide and

(1S,2S)-N-[(1S)-2-amino-2-oxo-1-phenylethyl]-2-[4-(1H-indazol-3-ylcarbonyl)-1-piperazinyl]cyclohexanecarboxamide

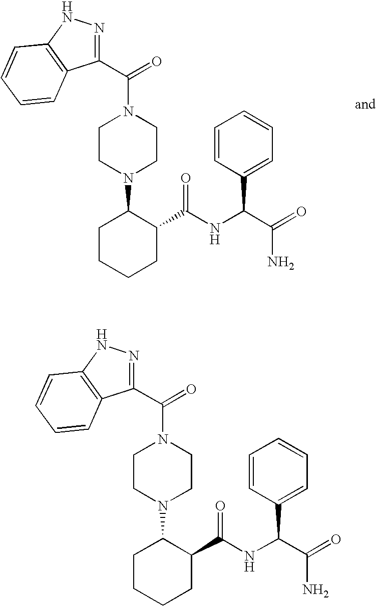

146 mg (about 0.19 mmol) of the carboxylic acid from step 3c), 37.2 mg (0.28 mmol) of HOBT, and 55.1 mg (0.29 mmol) of EDC are initially charged in DMF (3 ml). At RT, 46.7 mg (0.25 mmol) of (S)-phenylglycinamide hydrochloride, 0.16 ml (1.50 mmol) of N-methylmorpholine and a spatulatip of DMAP are added, and the reaction mixture is stirred at RT overnight. Water is added, the mixture is stirred for 2 h and the resulting precipitate is filtered off and washed with water. The solid is dried under reduced pressure and then triturated with diethyl ether for 1 h. Following, filtration and washing with diethyl ether, the product is again dried under reduced pressure. 44 mg (46% of theory) of crystalline product and 10 mg of mother liquor material are isolated.

R_{f} (methanol/dichloromethane 1:10) 0.27

HPLC (method A)=3.66+3.83 main.
Step 3e) (Separation of diastereomers): 1-[(1R,2R)-2-({[(1S)-2-Amino-2-oxo-1-phenylethyl]amino}carbonyl)cyclohexyl]-4-(1H-indazol-3-ylcarbonyl)piperazin-1-ium trifluoroacetate(diastereomer 3A) 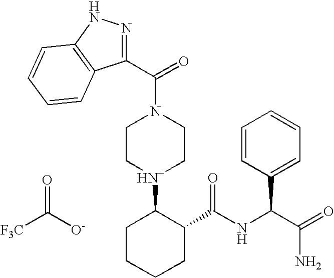
and
1-[(1S,2S)-2-({[(1S)-2-amino-2-oxo-1-phenylethyl]amino}carbonyl)cyclohexyl]-4-(1H-indazol-3-ylcarbonyl)piperazin-1-ium trifluoroacetate(diastereomer 3B)

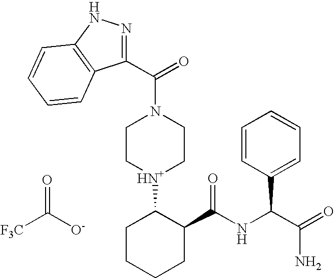

44 mg of the mixture of diastereomers from step 3d) are separated by preparative HPLC (Kromasil 100 C 18, 7 μm, 250×20 mm, 40° C., injection volume=0.75 ml, flow rate=25 ml/min, 0.2% strength aqueous trifluoroacetic acid/acetonitrile 95:5 to 5:95 over 10 min). This gives 16 mg (29% of theory) of diastereomer 3A and 18 mg (33% of theory) of diastereomer 3B.

Diastereomer 3A:

MS (ESI pos): m/z=489 (M+H)⁺

¹H-NMR (200 MHz, DMSO-D₆): δ=1.00-1.58 (m, 4H), 1.58-2.18 (m, 4H), 2.60-4.30 (br. m, 12H), 5.39 (d, 1H, J=7.3 Hz), 7.00-7.32 (m, 4H), 7.32-7.52 (m, 3H), 7.65 (d, 1H), 7.75 (br. s, 1H), 8.04 (d, 1H), 8.88 (d, 1H), 13.62 (br. s, 1H).

Diastereomer 3B:

MS (ESI pos): m/z=489 (M+H)⁺

¹H-NMR (200 MHz, DMSO-d₆): δ=1.00-1.56 (m, 4H), 1.58-1.74 (br. d, 1H), 1.74-1.96 (m, 2H), 2.00-2.24 (m, 1H), 2.66-2.93 (br. s, 1H), 3.00-4.24 (br. m, 10H), 5.31 (d, 1H, J=5.9 Hz), 7.25 (t, 1H), 7.31-7.50 (m, 6H), 7.55 (br. s, 1H), 7.65 (d, 1H), 7.91 (br. s, 1H), 8.05 (d, 1H), 8.90 (br. s, 1H), 13.64 (br. s, 1H).

### Example 4

(1R,2R)-N-[(1S)-2-Amino-1-(4-fluorophenyl)-2-oxoethyl]-2-{4-[(4-methylphenyl)-sulphonyl]-1-piperazinyl}cyclohexanecarboxamide and

(1S,2S)-N-[(1S)-2-amino-1-(4-fluorophenyl)-2-oxoethyl]-2-{4-[(4-methylphenyl)-sulphonyl]-1-piperazinyl}cyclohexanecarboxamide 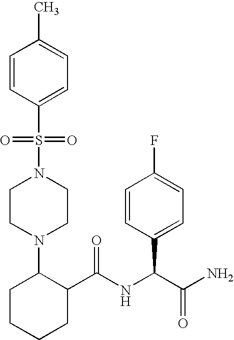
Step 4a):
Ethyl (1 R*,2R*)-2-{4-[(4-methylphenyl)sulphonyl]-1-piperazinyl}cyclohexane-carboxylate

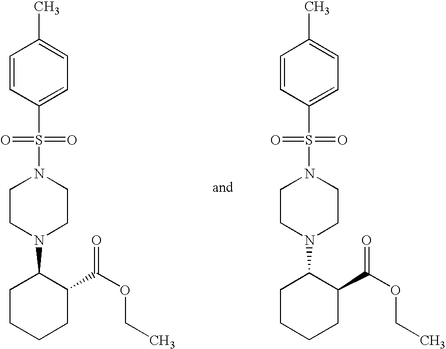

200 mg (0.96 mmol) of ethyl trans-2-amino-1-cyclohexanecarboxylate hydrochloride and 285 mg (0.96 mmol) of N,N-bis-(2-chloroethyl)toluenesulphonamide are dissolved in N-ethyldiisopropylamine (Hünig base) (1.7 ml) and initially heated at 130° C. for 3 h. Acetonitrile (5 ml) is then added, and the mixture is stirred at 70° C. overnight. The reaction mixture is then allowed to stand at RT for 3 d. For work-up, the mixture is extracted with dichloromethane and 0.1 N aqueous sodium hydroxide solution. After phase separation, the aqueous phase is reextracted with dichloromethane. The combined organic phases are dried over sodium sulphate, filtered and concentrated. The residue is dried chromatographically on silica gel using the mobile phase petroleum ether/ethyl acetate 4:1. This gives 96 mg (25% of theory) of the desired piperazine derivative.

R_{f} (methanol/dichloromethane 1:10)=0.35

HPLC (method C): Rₜ=4.01 min.

MS (ESI pos): m/z=395 (M+H)⁺

Step 4b):

(1 R*,2R*)-2-{4-[(4-Methylphenyl)sulphonyl]-1-piperazinyl}cyclohexanecarboxylic acid

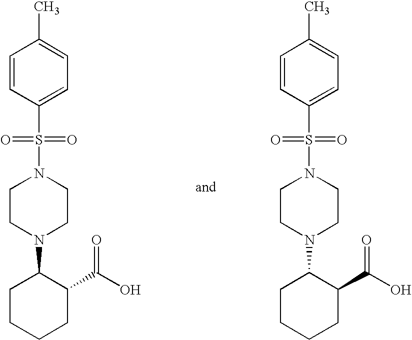

89 mg (0.22 mmol) of the ethyl ester from step 4a) are dissolved in methanol (10 ml), 5 N aqueous sodium hydroxide solution (1 ml) is added and the reaction mixture is heated at reflux overnight. The solution is neutralized with hydrochloric acid and extracted with water and dichloromethane. The phases are separated and the aqueous phase is reextracted twice with dichloromethane. The combined organic phases are dried over sodium sulphate, filtered and concentrated. This gives 63 mg (76% of theory) of crude product which is reacted further without further purification.

R_{f} (methanol/dichloromethane 1:10)=0.53

HPLC (method C): Rₜ=3.31 min.

MS (ESI pos): m/z=367 (M+H)⁻

Step 4c):

(1R,2R)-N-[(1S)-2-Amino-1-(4-fluorophenyl)-2-oxoethyl]-2-{4-[(4-methylphenyl)-sulphonyl]-1-piperazinyl}cyclohexanecarboxamide and

(1S,2S)-N-[(1S)-2-amino-1-(4-fluorophenyl)-2-oxoethyl]-2-{4-[(4-methylphenyl)-sulphonyl]-1-piperazinyl}cyclohexanecarboxamide

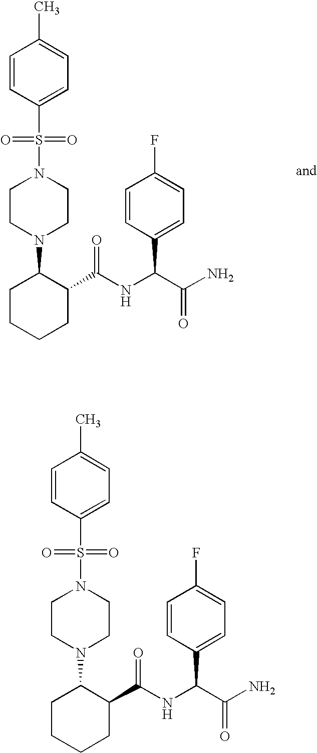

Under argon, 79 mg (0.22 mmol) of the carboxylic acid from step 4b), 32 mg (0.24 mmol) of HOBt and 48 mg (0.25 mmol) of EDC are initially charged in DMF (3 ml) at RT, 44 mg (0.22 mmol) of (S)-4-fluorophenylglycinamide hydrochloride, 66 mg (0.65 mmol) of N-methylmorpholine and a spatulatip of 4-dimethylaminopyridine are added and the reaction mixture is stirred at RT overnight. Because of incomplete conversion, another 66 mg of N-methylmorpholine are added, and the mixture is allowed to stand at RT for three days. The reaction mixture is concentrated and extracted with dichloromethane and water, and the organic phase is dried over sodium sulphate, filtered and concentrated. The residue is purified chromatographically on silica gel using the mobile phase methanol/dichloromethane 1:10. This gives 61 mg (55% of theory) of the desired product as a mixture of diastereomers.

R_{f} (methanol/dichloromethane 1:10)=0.37 and 0.41

HPLC (method B): Rₜ=3.99 min. and 4.06 min.

MS (ESI pos): m/z=517 (M+H)⁺

### Example 5

(1R,2R)-N-[(1S)-2-Amino-1-(4-fluorophenyl)-2-oxoethyl]-2-[4-(3-pyridinylsulphonyl)-1-piperazinyl]cyclohexanecarboxamide and

(,1S,2S)-N-[(1S)-2-amino-1-(4-fluorophenyl)-2-oxoethyl]-2-[4-(3-pyridinylsulphonyl)-1-piperazinyl]cyclohexanecarboxamide 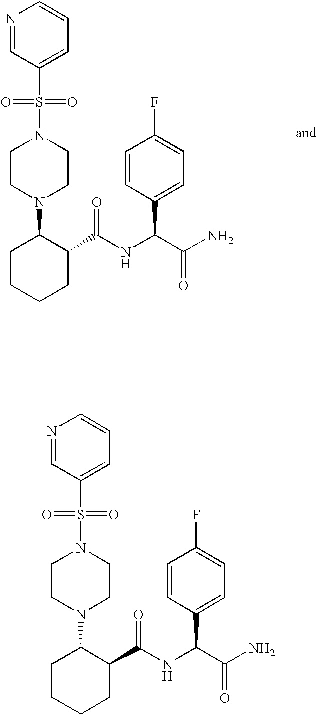
Step 5a):
tert-Butyl(1R*,2R*)-2-[4-(3-pyridinylsulphonyl)-1-piperazinyl]cyclohexanecarboxylate

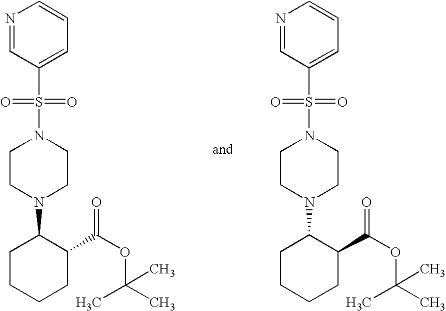

219 mg (0.76 mmol) of the piperazine from Example 1/step 1d) and 0.23 ml (1.66 mmol) of triethylamine are initially charged in dichloromethane (7 ml) and 162 mg (0.76 mmol) of 3-pyridinesulphonyl chloride hydrochloride are added at RT, rinsing with 3 ml of dichloromethane. The reaction mixture is stirred at RT overnight and allowed to stand at RT for 3 d. The solvent is removed under reduced pressure and the residue is purified chromatographically on silica gel using the mobile phase methanol/dichloromethane 1:10. This gives 201 mg (65% of theory) of the product.

R_{f} (methanol/dichloromethane 1:10)=0.73

HPLC (method B): Rₜ=3.88 min.

MS (ESI pos): m/z=410 (M+H)⁺

Step 5b):

1-[(1R*,2R*)-2-Carboxycyclohexyl]-4-(3-pyridiniumylsulphonyl)piperazin-1-ium bis(trifluoroacetate)

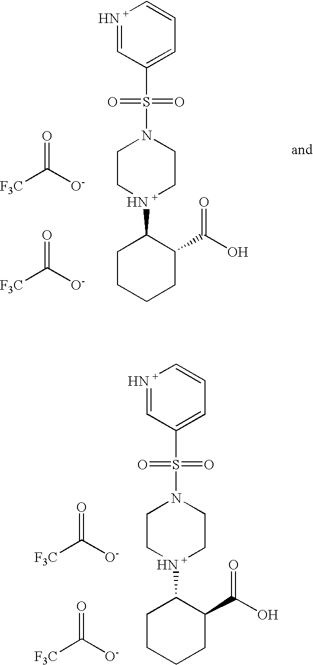

180 mg (0.44 mmol) of the tert-butyl ester from step 5a) aide dissolved in dichloromethane (4 ml), and trifluoroacetic acid (2 ml) is added at RT. The reaction mixture is stirred at RT for 2 h and then concentrated using a rotary evaporator, and the residue is taken up in dichloromethane and reconcentrated to dryness. The residue is dried under reduced pressure. This gives 325 mg (96% of theory) of the crude product of a purity of 76% by HPLC which is reacted without further purification.

R_{f} (methanol dichloromethane 1:10)=0.38

HPLC (method B): Rₜ=3.08 min.

MS (ESI pos): m/z=354 (M+H)⁺

Step 5c):

(1R,2R)-N-[(1S)-2-Amino-1-(4-fluorophenyl)-2-oxoethyl]-2-[4-(3-pyridinylsulphonyl)-1-piperazinyl]cyclohexanecarboxamide and

(1S,2S)-N-[(1S)-2-amino-1-(4-fluorophenyl)-2-oxoethyl]-2-[4-(3-pyridinylsulphonyl)-1-piperazinyl]cyclohexanecarboxamide

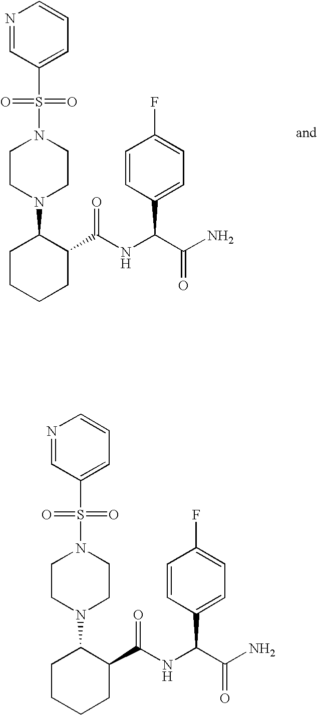

128 mg (0.22 mmol) of the carboxylic acid from step 5b), 33 mg (0.24 mmol) of HOBt and 49 mg (0.25 mmol) of EDC are initially charged in anhydrous DMF (2.5 ml), 45 mg (0.22 mmol) of (S)-4-fluorophenylglycinamide hydrochloride, 0.15 ml (1.32 mmol) of N-methylmorpholine and a spatulatip of DMAP are added at RT and the reaction mixture is stirred overnight. The mixture is allowed to stand at RT for 2 days and then extracted with dichloromethane and water, the aqueous phase is extracted twice with dichloromethane and the combined organic phases are dried over sodium sulphate, filtered and concentrated to dryness. This gives 148 mg of crude product which is purified chromatographically on silica gel using the mobile phase methanol/dichloromethane 1:10. The product fraction is triturated with diethyl ether and the crystalline product is filtered off with suction and dried. This gives 63 mg (57% of theory) of the desired product as a 1:1 mixture of diastereomers and 29 mg of product-containing mother liquor material.

R_{f} (methanol/dichloromethane 1:10)=0.37

HPLC (method B): Rₜ=3.41+3.54 min.

MS (ESI pos): m/z=504 (M+H)⁺

### Example 6

(1 R,2R)-N-[(1S)-2-Amino-2-oxo-1-phenylethyl]-2-[4-(3-pyridinylsulphonyl) 1-piperazinyl]cyclohexanecarboxamide and

(1S,2S)-N-[(S)-2-amino-2-oxo-1-phenylethyl]-2-[4-(3-pyridinylsulphonyl)-1-piperazinyl]cyclohexanecarboxamide

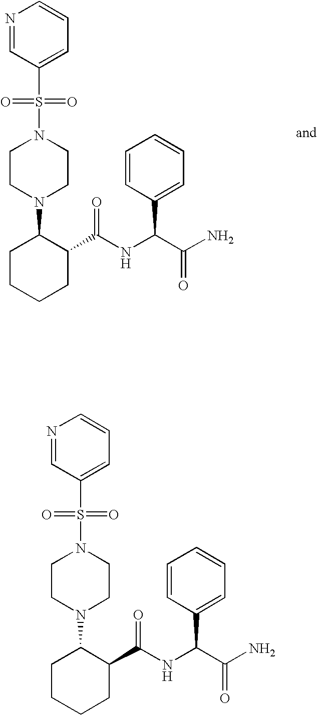

These compounds are prepared analogously to Example 5 by reacting the carboxylic acid from step 5b) with 41 mg (0.22 mmol) of (S)-phenylglycinamide hydrochloride. 54 mg (51% of theory) of the desired product are isolated as a mixture of diastereomers.

R_{f} (methanol/dichlorormethane 1:10)=0.41

HPLC (method B): Rₜ=3.33+3.45 min.

MS (ESI pos): m/z=486 (M+H)⁺

### Example 7

(1R,2R)-N-[(1S)-2-Amino-2-oxo-1-phenylethyl]-2-(4-benzyl-1-piperazinyl)-cyclohexanecarboxamide and

(1S,2S)-N-[(1S)-2-amino-2-oxo-1-phenylethyl]-2-(4-benzyl-1-piperazinyl)-cyclohexanecarboxamide 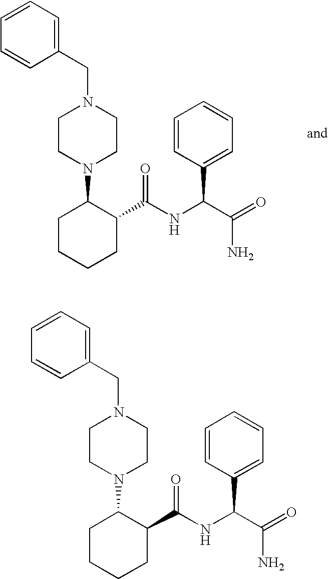
Step 7a):
1-Benzyl-4-[(1 R*,2R*)-2-carboxycyclohexyl]piperazinediium bis(trifluoroacetate)

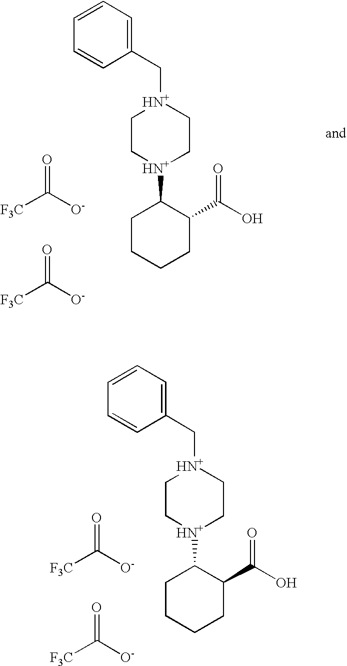

2.43 g (6.8 mmol) of the tert-butyl ester from Example 1/step 1c) are dissolved in dichloromethane (20 ml) and trifluoroacetic acid (10 ml) is added at RT. After 2.5 h of stirring at RT, a further 10 ml of trifluoroacetic acid are added, and the reaction mixture is stirred at RT for 5 h. The mixture is concentrated to dryness using a rotary evaporator and the residue is twice taken up in dichloromethane and reconcentrated and dried under reduced pressure. This gives 5.15 g of crude product which is reacted further without purification.

R_{f} (methanol/dichloromethane 1:10)=0.30

HPLC (method A): Rₜ 3.26 min.

MS (ESI pos): m/z 303 (M+H)⁺, 325 (M+Na)⁺

Step 7b):

1-Benzyl-4-[(1R*,2R*)-2-carboxycyclohexyl]piperazinediium dichloride

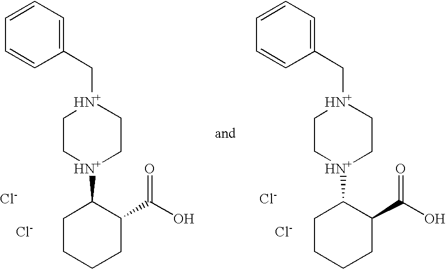

20.0 g (55.8 mmol) of the compound from Example 1/Step 1c) are dissolved in dichloromethane (200 ml), 80 ml (320 mmol) of a 4 M HCl solution in dioxane are added and the mixture is stirred at room temperature overnight. Another 80 ml (320 mmol) of 4 M HCl solution in dioxane and dichloromethane (135 ml) are added, and the mixture is stirred for 24 h. The resulting precipitate is filtered off with suction, washed with diethyl ether and dried under reduced pressure. This gives 21.4 g (100% of theory) of a colourless solid.

HPLC (method A): Rₜ=3.22 min.

MS (ESI pos): m/z=303 (M+H)⁺

¹H-NMR (300 MHz, DMSO-d₆): δ=1.10-1.49 (m, 4H), 1.60 (br. d, 1H), 1.77 (br. d, 1H), 1.95 (br. t, 2H), 2.56 (br. t, 1H), 3.14 (br. s, 4H), 3.37 (br. d, 4H), 3.96 (br. s), 4.32 (s, 2H), 7.43-7.50 (m, 3H), 7.57-7.65 (m, 2H), 11.45 (br. s, 1H).

Step 7c):

Diastereomer mixture of (1R,2R)-N-[(1S)-2-amino-2-oxo-1-phenylethyl]-2-(4-benzyl-1-piperazinyl)-cyclohexanecarboxamide and

(1S,2S)-N-[(1S)-2-amino-2-oxo-1-phenylethyl]-2-(4-benzyl-1-piperazinyl)-cyclohexanecarboxamide 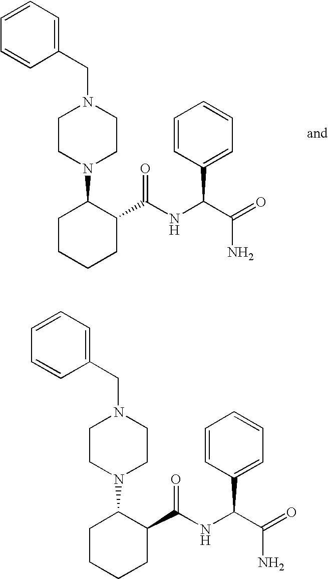
Method A:

149 mg (0.16 mmol at a purity of: 58%) of the carboxylic acid from step 7a), 42 mg (0.31 mmol) of HOBt and 62 mg (0.32 mmol) of EDC are initially charged in anhydrous DMF (3 ml) and, at RT, 52 mg (0.28 mmol) of (S)-phenylglycinamide hydrochloride, 0.18 ml (1.68 mmol) of N-methylmorpholine and a spatulatip of DMAP are added. The reaction mixture is stirred for 2 days and allowed to stand at RT for 2 days. The mixture is extracted with dichloromethane and water, the aqueous phase is reextracted five times with dichloromethane and the combined organic phases are dried over sodium sulphate, filtered and concentrated to dryness using a rotary evaporator. The crude product is purified by preparative HPLC and separated into the two diastereomers (see step 7d).

Method B:

21.5 g (57.3 mmol) of the carboxylic acid from step 7b), 8.51 g (63.0 mmol) of HOBt and 12.6 g (65.9 mmol) of EDC are initially charged in DMF (270 ml). At RT, 34.8 g (344 mmol) of N-methylmorpholine and a spatulatip of DMAP are added, and the reaction mixture is stirred at RT for 3 days. Water (1.4l) is added to the solution, which is then adjusted to pH 9 using aqueous potassium carbonate solution and extracted three times with ethyl acetate (420 ml each). The combined organic phases are washed twice with buffer solution [CertiPUR pH 9 (boric acid, potassium chloride, sodium hydroxide)] (102 ml each), dried over sodium sulphate and filtered, and the solvent is removed under reduced pressure. The residue is purified chromatographically on silica gel (850 g; 0.063-0.2 mm) using the mobile phase dichloromethane/methanol 95:5 (5.7 l) and 9:1 (3.5 l). This gives 20.0 g (79% of theory) of the mixture of diastereomers.

HPLC (method A): Rₜ=3.54 min+3.63 min.

MS (ESI pos): m/z=435 (M+H)⁺

¹H-NMR (300 MHz, CDCl₃): δ=1.01-1.42 (m, 8H), 1.64-1.85 (br. m, 4H), 1.85-2.00 (br. m, 3H), 2.10-2.61 (m, 18H), 2.62-2.84 (m, 5H), 3.34 (s, 2H), 3.39+3.46 (each d, 2H), 5.57 (dd, 2H), 5.64 (br. s, 2H), 6.54+6.64 (each br. s, 2H), 7.20-7.39 (m, 16H), 7.40-7.48 (m, 4H), 9.70 (d, 1H), 9.78 (d, 1H).

Step 7d) (Separation of Diastereomers):

(1R,2R)-N-[(1S)-2-Amino-2-oxo-1-phenylethyl]-2-(4-benzyl-1-piperazinyl)-cyclohexanecarboxamide (diastereomer 7A) 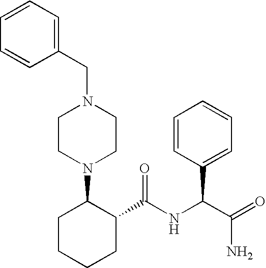
and
(1 S,2S)-N-[(1S)-2-amino-2-oxo-1-phenylethyl]-2-(4-benzyl-1-piperazinyl)-cyclohexanecarboxamide (diastereomer 7B)

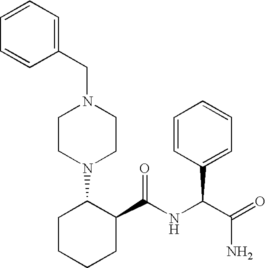

The crude product (150 mg) from step 7c) is purified by preparative HPLC on polyamine II (YMC Pack, 5 μm, 250×20 mm, 30° C., injection volume=0.4 ml, flow rate=25 ml/min) using isohexane/ethanol 93:7 and separated into the diastereomers. This gives 21 mg (30% of theory) of diastereomer 7A and 25 mg (35% of theory) of diastereomer 7B.

Diastereomer 7A:

R_{f} (methanol/dichloromethane 1:10)=0.32

HPLC (method see separation method using a 250×4.6 mm column, flow rate

1 ml/min, isohexane/ethanol 90:10): Rₜ=6.98 min.

Diastereomer 7B:

R_{f} (methanol/dichloromethane 1:10)=0.32

HPLC (method see diastereomer 7A): Rₜ=6.27 min.

### Example 8

(1R,2R)-N-[(1S)-2-Amino-1-(4-fluorophenyl)-2-oxoethyl]-2-(4-benzyl-1-piperazinyl)cyclohexanecarboxamide(diastereomer 8A) 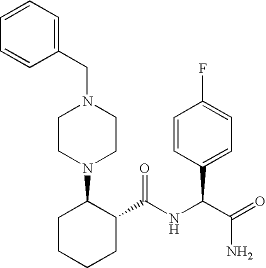
and
(1S,2S)-N-[(1S)-2-amino-1-(4-fluorophenyl)-2-oxoethyl]-2-(4-benzyl 1-piperazinyl)-cyclohexanecarboxamide (diastereomer 8B)

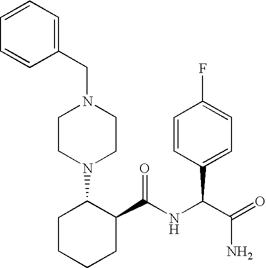

The compound from Example 7/step 7a) is reacted analogously to step 7b) using 52 mg (0.28 mmol) of (S)-4-fluorophenylglycinamide hydrochloride instead of (S)-phenylglycinamide hydrochloride, and the product is then, analogously to step 7c), separated into the diastereomers using isohexane/ethanol 90:10. This gives 6 mg each (8% of theory) of the two diastereomers.

Mixture of Diastereomers:

R_{f} (methanol/dichloromethane 1:10)=0.32

HPLC (method B): Rₜ=3.54+3.62 min.

MS (ESI pos): m/z=453 (M+H)⁺, 475 (M+Na)⁺

Diastereomer 8A:

HPLC (method see diastereomer 7A): Rₜ=6.92 min.

Diastereomer 8B:

HPLC (method see diastereomer 7A): Rₜ=6.11 min.

### Example 9

(1R,2R)-N-[(1S)-2-Amino-2-oxo-1-phenylethyl]-2-[4-(3-quinolinylmethyl)-1-piperazinyl]cyclohexanecarboxamide and

(1S,2S)-N-[(1S)-2-amino-2-oxo-1-phenylethyl]-2-[4-(3-quinolinylmethyl)-1-piperazinyl]cyclohexanecarboxamide 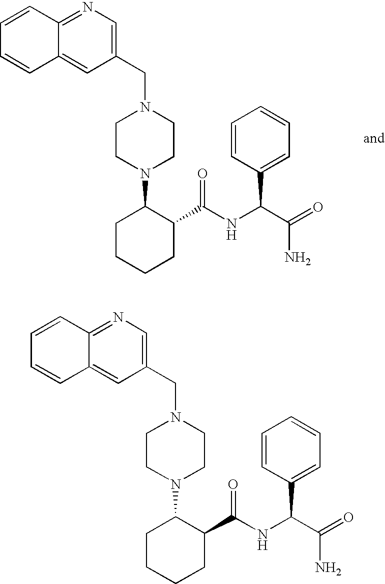
Step 9a):
tert-Butyl (1 R*,2R*)-2-[4-(3-quinolinylmethyl)-1-piperazinyl]cyclohexanecarboxylate

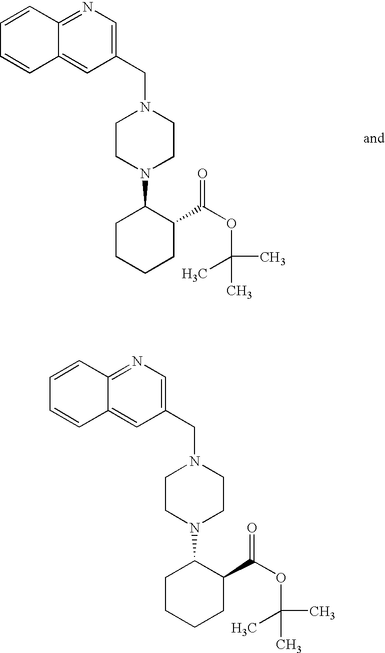

At RT, 712 mg (3.36 mmol) of sodium triacetoxyborohydride are added a little at a time to a solution of 301 mg (1.12 mmol) of piperazine from Example 1/step 1d) and 176 mg (1.12 mmol) of 3-quinolinecarboxaldehyde in methanol (5 ml) and acetic acid (0.5 ml). The reaction mixture is stirred at RT overnight and then concentrated and the residue is taken up in dichloromethane and extracted with 0.1 N aqueous sodium hydroxide solution. The aqueous phase is reextracted twice with dichloromethane and the combined organic phases are dried over sodium sulphate, filtered and concentrated under reduced pressure. The crude product (450 mg) is twice purified chromatographically on silica gel using the mobile phase methanol/dichloromethane 1:10. This gives 183 mg (40% of theory) of product.

R_{f} (methanol/dichloromethane 1:10)=0.50

HPLC (method C): Rₜ=3.32 min.

MS (ESI pos): m/z=410 (M+H)⁺

Step 9b):

3-({4-[(1R*,2R*)-2-Carboxycyclohexyl]-1-piperazinediiumyl}methyl)quinolinium tris(trifluoroacetate)

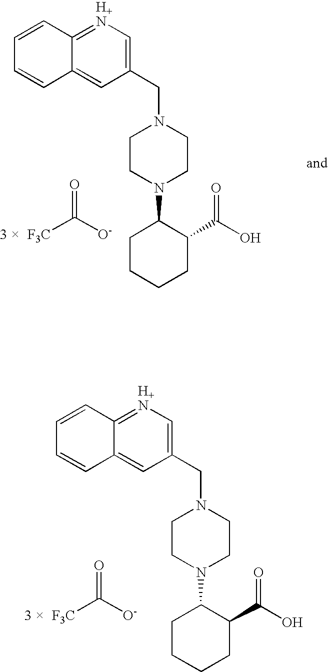

146 mg (0.36 mmol) of the tert-butyl ester from step 9a) are dissolved in dichloromethane (4 ml), trifluoroacetic acid (2 ml) is added at RT and the mixture is stirred at RT for 3 h. The reaction mixture is concentrated to dryness, taken up in dichloromethane and reconcentrated to dryness. The residue is once more stirred with dichloromethane (6 ml) and trifluoroacetic acid (3 ml) at RT for 3 h and worked up as described above. This gives 341 mg of an oily product which is reacted without further purification.

R_{f} (methanol/dichloromethane 1:10)=0.05

HPLC (method A): Rₜ=3.27 min.

MS (ESI pos): m/z=354 (M+H)⁺

Step 9c):

Diastereomer mixture of (1R,2R)-N-[(1S)-2-amino-2-oxo-1-phenylethyl]-2-[4-(3-quinolinylmethyl)-1-piperazinyl]cyclohexanecarboxamide and

(1S,2S)-N-[(1S)-2-amino-2-oxo-1-phenylethyl]-2-[4-(3-quinolinylmethyl)-1-piperazinyl]cyclohexanecarboxamide

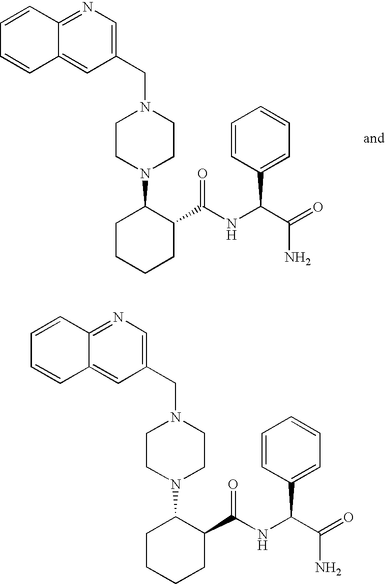

169 mg (0.18 mmol at a purity of 74%) of the product from step 9b), 27 mg (0.20 mmol) of HOBt and 40 mg (0.21 mmol) of EDC are initially charged in anhydrous DMF (2 ml), and 34 mg (0.18 mmol) of (S)-phenylglycinamide hydrochloride, 0.12 ml (1.08 mmol) of N-methylmorpholine and a spatulatip of DMAP are added. The reaction mixture is stirred at RT overnight, water (10 ml) is added and the mixture is extracted three times with dichloromethane. The combined organic phases are dried over sodium- sulphate and filtered and the solvent is removed under reduced pressure. The crude product (111 mg) is twice purified chromatographically on silica gel using the mobile phase methanol/dichloromethane 1:10. 39 mg (45% of theory) of the desired product are isolated as a 1:1 mixture of diastereomers.

R_{f} (methanol/dichloromethane 1:10)=0.24

HPLC (method A): Rₜ=3.48+3.64 min.

MS (DCI/NH₃): m/z=486 (M+H)⁺

### Example 10

Diastereomer mixture of (1R,2R)-N-[(1S)-2-amino-1-(4-fluorophenyl)-2-oxoethyl]-2-[4-(3-quinolinylmethyl)-1-piperazinyl]cyclohexanecarboxamide and

(1S,2S)-N-[(1S)-2-amino-1-(4-fluorophenyl)-2-oxoethyl]-2-[4-(3-quinolinylmethyl)-1-piperazinyl]cyclohexanecarboxamimde

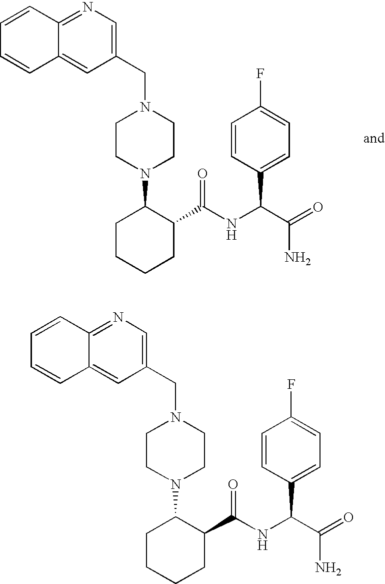

Example 10 is prepared analogously to Example 9/step 9c) using 37 mg (0.18 mmol) of (S)₄-fluorophenylglycinamide hydrochloride instead of (S)-phenylglycinamide hydrochloride. This gives 35 mg (39% of theory) of the desired product as a 1:1 mixture of diastereomers.

R_{f} (methanol/dichloromethane 1:10)=0.26

HPLC (method A): Rₜ=3.58+3.73 min.

MS (ESI pos): m/z=504 (M+H)⁺

### Example 11

trans-N-[(1S)-1-(Aminocarbonyl)-3-(methylsulphonyl)propyl]-2-(4-benzyl-1-piperazinyl)cyclohexanecarboxamide 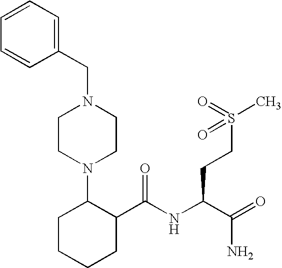
Step 11a):
1-Benzyl-4-[(1R*,2R*)₂carboxycyclohexyl]piperazinediium dichloride

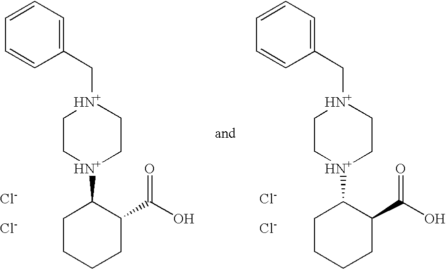

1.00 g (2.80 mmol) of the tert-butyl ester from Example 1/step 1 c) is dissolved in dioxane (5 ml). At room temperature, 2.8 ml. (11.2 mmol) of a 4 M solution of HCl gas in dioxane are added, and the mixture is then stirred overnight. The resulting solid is filtered off with suction, washed with diethyl ether and dried under reduced pressure. It is then suspended in dichloromethane (5 ml) and, at room temperature, initially stirred overnight with 2.8 ml (11.2 mmol) of a 4 M solution of HCl gas in dichloromethane and then again overnight with a further 1 ml (4 mmol) of 4 M HCl in dichloromethane. The crystalline solid is filtered off, washed with diethyl ether and dried under reduced pressure. This gives 859 mg (74% of theory) of the desired product.

R_{f} (methanol/dichloromethane 1:10)=0.31

HPLC (method A): Rₜ=3.15 min.

MS (ESI pos.): m/z=303 (M+H)⁺

Step 11b):

trans-N-[(11S)-1-(Aminocarbonyl)-3-(methylsulphonyl)propyl]-2-(4-benzyl-1-piper-azinyl)cyclohexanecarboxamide

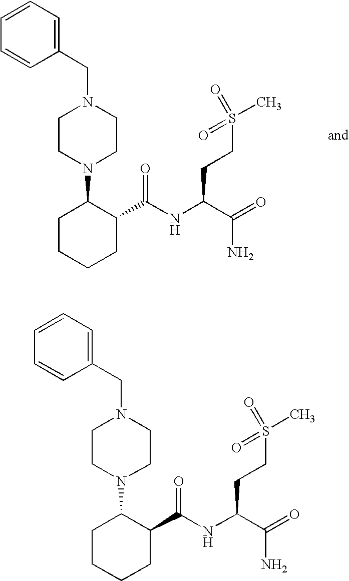

Analogously to the procedure of Example 1/step 1 g), 855 mg (2.28 mmol) of the carboxylic acid from step 11a), 494 mg (2.28 mmol) of S,S-dioxo-L-methioninamide hydrochloride, 339 mg (2.51 mmol) of HOBt, 503 mg (2.62 mmol) of EDC, 1.5 ml (13.7 mmol) of N-methylmorpholine and a spatulatip of DMAP are reacted in DMF (10 ml) at room temperature overnight. The mixture is extracted with water and dichloromethane, the aqueous phase is extracted with dichloromethane and the combined organic phases are dried over sodium sulphate, and the isolated crude product (1.18 g) is then purified chromatographically on silica gel using the mobile phase methanol/dichloromethane 1:10. This gives 752 mg (71% of theory) of crystalline product.

R_{f} (methanol/dichloromethane)=0.16

HPLC (method A): Rₜ=3.14 min.

MS (ESI pos.): m/z=465 (M+H)⁺

### Example 12

4-[(1R,2R)-2-({[(1S)-2-Amino-2-oxo-1-phenylethyl]amino}carbonyl)cyclohexyl]-N-(4-fluorophenyl)-1-piperazinecarboxamide and

4-[(1 S,2S)-2-({[(1S)-2-amino-2-oxo-1-phenylethyl]amino)}carbonyl)cyclohexyl]-N-(4-fluorophenyl)-1-piperazinecarboxamide 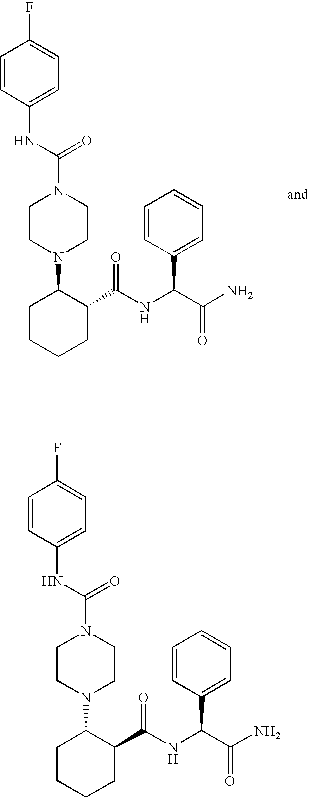
Step 12a):
tert-Butyl (1R*,2R*)-2-(4-{[(4-fluorophenyl)amino]carbonyl}-1-piperazinyl)cyclohexanecarboxylate

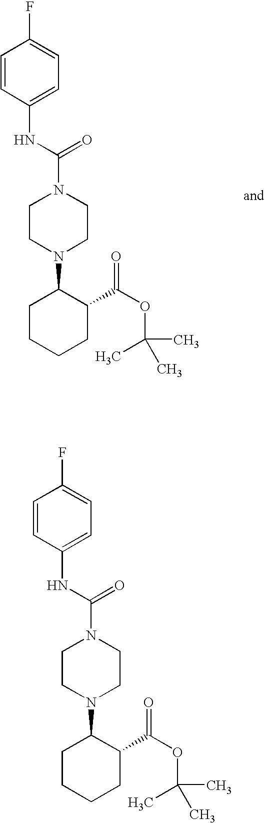

99 mg (0.37 mmol) of the piperazine from Example 1/step 1d) and 51 mg (0.37 mmol) of 4-fluorophenyl isocyanate are initially charged in toluene (3 ml) and stirred at 60° C. for 3 h. The solvent is removed under reduced pressure and the residue (213 mg) is twice purified chromatographically on silica gel, initially using the mobile phase methanol/dichloromethane 1:20 and then using methanol/dichloromethane 1:10. This gives 160 mg (81% of theory) of the product of a purity of 76% by HPLC which is reacted without further purification.

R_{f} (methanol/dichloromethane 1:10) 0.71

HPLC (method A): Rₜ=4.33 min.

MS (ESI pos): m/z=406 (M+H)⁺, 428 (M+Na)+

Step 12b):

1-[(1R*,2R*)-2-Carboxycyclohexyl]-4-{[(4-fluorophenyl)amino]carbonyl}piperazin-1-ium trifluoroacetate

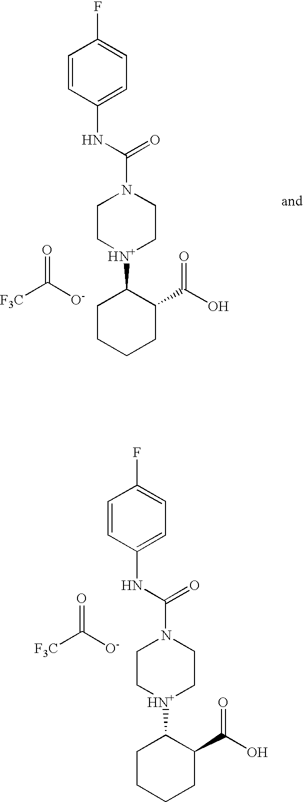

150 mg (0.37 mmol) of the tert-butyl ester from step 12a) are initially charged in dichloromethane (4 ml), trifluoroacetic acid (2 ml) is added at RT and the reaction mixture is stirred at RT for 6 h. The mixture is concentrated to dryness using a rotary evaporator and the residue is taken up in dichloromethane, reconcentrated and dried under reduced pressure. This gives 216 mg (89% of theory) of crude product of a purity of 71% by HPLC which is reacted further without purification.

R_{f} (methanol/dichloromethane 1:10)=0.12

HPLC (method A): Rₜ=3.65 min.

MS (ESI pos): m/z=350 (M+H)⁺

Step 12c):

Diastereomer mixture of 4-[(1R,2R)-2-({[(1S)-2-amino-2-oxo-1-phenylethyl]amino}carbonyl)cyclohexyl]-N-(4-fluorophenyl)-1-piperazinecarboxamide and

4-[(1S,2S)-2-({[(1S)-2-amino-2-oxo-1-phenylethyl]amino}carbonyl)cyclohexyl]-N-(4-fluorophenyl)-1-piperazinecarboxamide

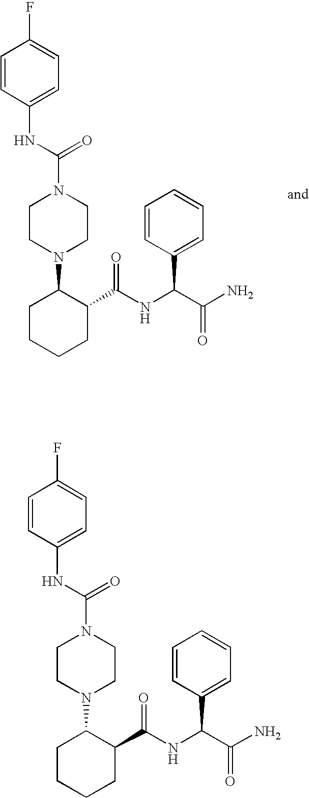

86 mg (0.19 mmol) of the carboxylic acid from step 12b), 28 mg (0.20 mmol) of HOBt and 41 mg (0.21 mmol) of EDC are initially charged in anhydrous DMF (2 ml), 35 mg (0.19 mmol) of (S)-phenylglycinamide hydrochloride, 0.12 ml (1.11 mmol) of N-methylmorpholine and a spatulatip of DMAP are added and the reaction mixture is stirred at RT overnight. For work-up, the mixture is extracted with water and dichloromethane, the aqueous phase extracted twice with dichloromethane, the combined orgamic phases are dried over sodium sulphate and filtered and the solvent is removed under reduced pressure. The residue (122 mg) is purified chromatographically on silica gel using the mobile phase methanol/dichloromethane. This gives 60 mg (67% of theory) of the desired product as a mixture of diastereomers.

R_{f} (methanol/dichloromethane 1:10)=0.35

HPLC (method A): Rₜ=3.76+3.91 min.

MS (ESI pos): m/z 482 (M+H)³⁰

### Example 13

Diastereomer mixture of 4-[(1R,2R)-2-({[(1S)-2-amino-1-(4-fluorophenyl)-2-oxoethyl]amino}carbonyl)-cyclohexyl]-N-(4-fluorophenyl)-1-piperazinecarboxamide and

4-[(1S,2S)-2-({[(1S)-2-amino-1-(4-fluorophenyl)-2-oxoethyl]amino}carbonyl)-cyclohexyl]-N-(4-fluorophenyl)-1-piperazinecarboxamide

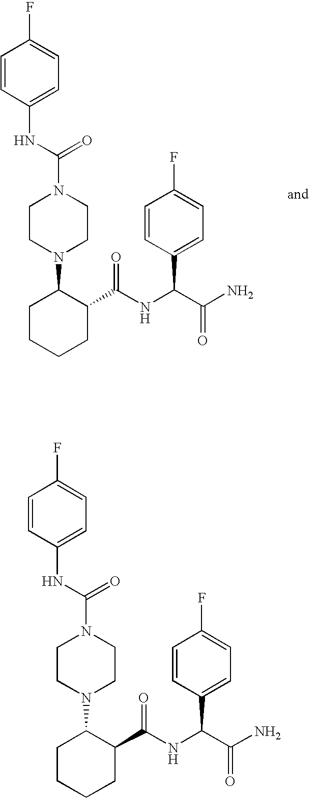

Example 13 is prepared analogously to Example 12/step 12 c) by reacting the carboxylic acid from step 12b) with 38 mg (0.19 mmol) of (S)-4-fluorophenyl-glycinamide hydrochloride instead of (S)-phenylglycinamide hydrochloride. This gives 57 mg (62% of theory) of the desired product as a mixture of diastereomers.

R_{f} (methanol/dichloromethane 1:10)=0.38

HPLC (method A): R. =3.84+3.98 min.

MS (ESI pos): m/z=500 (M+H)⁺

### Example 14

(1R,2R)-N-[(1S)-2-Amino-2-oxo-1-phenylethyl]-2-[4-(4-methoxybenzoyl)-1-piper-azinyl)cyclohexanecarboxamide 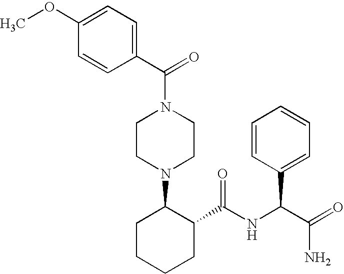
Step 14a):
Diastereomer mixture of (1R,2R)-N-[(1S)-2-amino-2-oxo-1-phenylethyl]-2-(1-piperazinyl)cyclohexanecarboxamide and
(1S,2S)-N-[(1S)-2-amino-2-oxo-1-phenylethyl]-2-(1-piperazinyl)cyclohexanecarboxamide

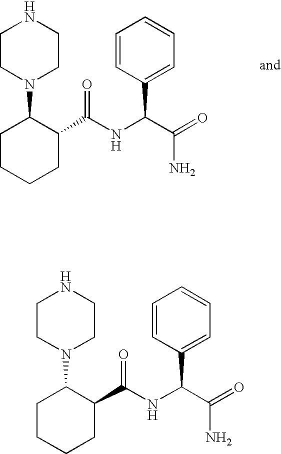

38.8 g (89.2 mmol) of the compound from Example 7/step 7c) are dissolved in ethanol (860 ml), and 7.8 g of 10% palladium on activated carbon are added under argon. The reaction mixture is, at RT and with constant stirring, hydrogenated under atmospheric pressure for 48 h. The mixture is filtered off with suction through kieselguhr, and the filtrate is concentrated to dryness. This gives 30.8 g (100% of theory) of the 1:1 mixture of diastereomers in a purity of 92% by HPLC.

Step 14b) (Separation of diastereomers):

(1R,2R)-N-[(1S)-2-Amino-2-oxo-1-phenylethyl]-2-(1-piperazinyl)cyclohexanecarboxamide (diastereomer 14b-A) 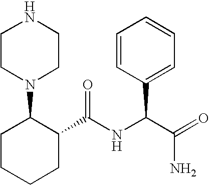
and
(1S,2S)-N-[(1S)-2-amino-2-oxo-1-phenylethyl]-2-(1-piperazinyl)cyclohexanecarboxamide (diastereomer 14b-B)

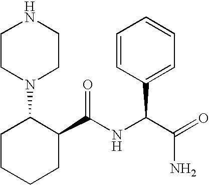

The crude diastereomer mixture (21.5 g) from step 14a) is purified and separated into the diastereomers by preparative HPLC (X-Terra RP 18-phase, 7 μm, 19×300 mm, RT, injection volume=0.375 ml, flow rate=25 ml/min, 0.2% strength aqueous trifluoroacetic acid/acetonitrile 8:2). The two fractions are each taken up in dichloromethane, extracted with aqueous sodium bicarbonate solution and adjusted to pH 10-11 using conc. aqueous ammonia solution. The phases are separated, the aqueous phase is reextracted twice with dichloromethane and the combined organic phases are dried over sodium sulphate. Filtration and removal of the solvent under reduced pressure give 7.1 g (32% of theory) of diastereomer 14b-A and 7.6 g (34% of theory) of diastereomer 14b-B.

Diastereomer 14b-A:

HPLC (method A): Rₜ=3.10 min.

MS (ESI pos): m/z=345 (M+H)⁺

¹H-NMR (300 MHz, CDCl₃): δ=1.04-1.35 (m, 4H), 1.66-1.86 (br. m, 2H), 1.87-1.99 (br. m, 1H), 2.20-2.35 (m, 2H), 2.36-2.63 (m, 6H), 2.70-2.93 (m, 6H), 5.57 (d, 1H), 5.76 (br. s, 1H), 6.37 (br. s, 1H), 7.29-7.39 (m, 3H), 7.39-7.47 (m, 2H), 9.73 (d, 1H).

Diastereomer 14b-B:

HPLC (method A): R. =3.31 min.

MS (ESI pos): m/z=345 (M+H)⁺

¹H-NMR (300 MHz, CDCl₃): δ=1.01-1.24 (m, 3H), 1.24-1.43 (m, 1H), 1.65-1.86 (br. m, 2H), 1.86-2.00 (br. m, 1H), 2.12-2.32 (m, 2H), 2.35-2.53 (m, 2H), 2.56-2.78 (m, 6H), 2.78-2.92 (m, 2H), 5.58 (d, 1H), 6.10 (br. s, 1H), 6.87 (br. s, 1H), 7.24-7.37 (m, 3H), 7.37-7.47 (m, 2H), 9.66 (d, 1H).

Step 14c):

(1R,2R)-N-[(1S)-2-Amino-2-oxo-1-phenylethyl]-2-[4-(4-methoxybenzoyl)-1-piperazinyl)cyclohexanecarboxamide

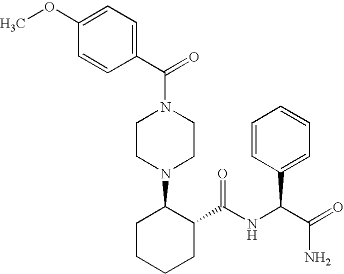

21.6 mg (0.16 mmol) of HOBt and 29.2 mg (0.15 mmol) of EDC are added to a solution of 50 mg (0.15 mmol) of the diastereomer 14b-A in DMF (5 ml). After 5 min of stirring at RT, 26.5 mg (0.17 mmol) of 4-methoxybenzoic acid, 0.06 ml (0.58 mmol) of 4-methylmorpholine and a spatulatip of 4-dimethylaminopyridine are added, and the mixture is stirred at RT overnight. The mixture is then separated by preparative RP-HPLC (column: YMC Gel ODS-AQ S-11 μm, 250×30 mm; mobile phase: acetonitrile/water; flow rate: 50 ml/min; UV detection at 210 nm). Concentration under reduced pressure gives 56 mg (80.6% of theory) of the product as a colourless solid.

HPLC (method A): Rₜ=3.71 min.

MS (ESI pos): m/z=479 (M+H)⁺

¹H-NMR (200 MHz, CD₃CN): δ=1.10-1.45 (m, 4H), 1.55-2.50 (m, 7H), 2.60-2.80 (m, 3H), 3.20-3.60 (m, 4H), 3.82 (s, 3H), 5.47 (d, 1H), 5.89 (br. s, 1H), 6.59 (br. s, 1H), 6.90-7.13 (m, 2H), 7.28-7.49 (m, 7H), 8.68 (d, 1H).

### Example 15

(1R,2R)-N-[(1S)-2-Amino-2-oxo-1-phenylethyl]-2-[4-(4-methylbenzoyl)-1-piper-azinyl)cyclohexanecarboxamide

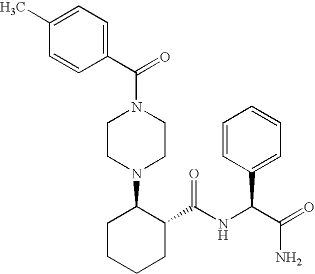

Analogously to the procedure for the preparation of Example 14, 100 mg (0.29 mmol) of the diastereomer 14b-A are reacted in DMF (4 ml) with 43.2 mg (0.32 mmol) of HOBt, 58.44 mg (0.30 mmol) of EDC, 47.4 mg (0.35 mmol) of 4-methylbenzoic acid, 0.13 ml (1.16 mmol) of 4-methylmorpholine and a spatulatip of 4-dimethylaminopyridine. Separation of the reaction mixture and concentration under reduced pressure gives 96 mg (71.5% of theory) of the product as a colourless solid.

HPLC (method A): Rₜ=3.84 min.

MS (ESI pos): m/z=436 (M+H)⁺

¹H-NMR (400 MHz, CD₃CN): δ=1.10-1.35 (m, 4H), 1.63-2.20 (m, 4H), 2.23-2.50 (m, 6H), 2.62-2.80 (m, 3H), 3.10-3.30 (br. m, 2H), 3.40-3.60 (br. m, 2H), 5.37 (d, 1H), 5.85 (br. s, 1H), 6.55 (br. s, 1H), 7.19-7.28 (m, 4H), 7.29-7.46 (m, 5H), 8.63 (br. d, 1H).

### Example 16

(1R,2R)-N-[(1S)-2-Amino-2-oxo-1-phenylethyl]-2-[4-(2,4-difluorobenzoyl)-1-piperazinyl)cyclohexanecarboxamide

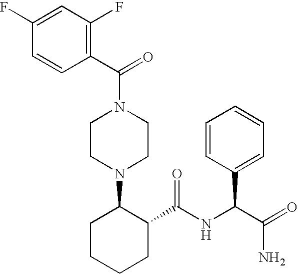

Analogously to the procedure for the preparation of Example 14, 50 mg (0.15 mmol) of the diastereomer 14b-A are reacted in DMF (2 ml) with 21.6 mg (0.16 mmol) of HOBt, 29.2 mg (0.15 mmol) of EDC, 27.5 mg (0.17 mmol) of 2,4-difluorbenzoic acid, 0.06 ml. (0.58 mmol) of 4-methylmorpholine and a spatulatip of 4-dimethylaminopyridine. Separation of the reaction mixture and concentration under reduced pressure gives 57 mg (81.3% of theory) of the product as a colourless solid.

HPLC (method A): Rₜ=3,79 min.

MS (ESI pos): m/z=485 (M+H)⁺

¹H-NMR (200 MHz, CD₃CN): δ=1.05-1.40 (m, 4H), 1.58-2.10 (m, 4H), 2.15-2.51 (m, 3H), 2.58-2.83 (m, 3H), 3.05-3.18 (br. m, 2H), 3.49-3.61 (br. m, 2H), 5.38 (d, 1H), 5.88 (br. s, 1H), 6.53 (br. s, 1H), 6.93-7.11 (m, 2H), 7.25-7.50 (m, 6H), 8.62 (br. d, 1H).

### Example 17

(1R,2R)-N-[(1S)-2-Amino-2-oxo-1-phenylethyl]-2-{4-[(5-methyl-2-thienyl)-carbonyl]-1-piperazinyl}cyclohexanecaiboxamide

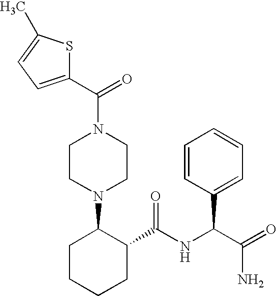

Analogously to the procedure for the preparation of Example 14, 50 mg (0.15 mmol) of the diastereomer 14b-A are reacted in DMF (2 ml) with 21.6 mg (0.16 mmol) of HOBt, 29.2 mg (0.15 mmol) of EDC, 24.8 mg (0.17 mmol) of 5-methylthiophene-2-carboxylic acid, 0.06 ml (0.58 mmol) of 4-methylmorpholine and a spatulatip of 4-dimethylaminopyridine. Separation of the reaction mixture and concentration under reduced pressure gives 58.8 mg (86.4% of theory) of the product as a colourless solid.

HPLC (method A): Rₜ=3.79 min.

MS (ESI pos): m/z 469 (M+H)⁺

¹H-NMR (200 MHz, CD₃CN): δ=1.05-1.40 (m, 4H), 1.55-2.10 (m, 4H), 2.20-2.52 (m, 6H), 2.51-2.81 (m, 3H), 3.45-3.63 (br. m, 4H), 5.38 (d, 1H), 5.89 (br. s, 1H), 6.57 (br. s, 1H), 6.75 (dd, 1H), 7.18 (d, 1H), 7.25-7.50 (m, 5H), 8.64 (br. d, 1H).

### Example 18

(1R,2R)-N-[(1S)-2-Amino-2-oxo-1-phenylethyl]-2-{4-[(2-pyrrolyl)carbonyl]-1-piperazinyl}cyclohexanecarboxamide

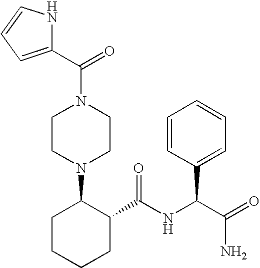

Analogously to the procedure for the preparation of Example 14, 50 mg (0.15 mmol) of the diastereomer 14b-A are reacted in DMF (2 ml) with 21.6 mg (0.16 mmol) of HOBt, 29.2 mg (0.15 mmol) of EDC, 19.4 mg (0.17 mmol) of pyrrole-2-carboxylic acid, 0.06 ml (0.58 mmol) of 4-methylmorpholine and a spatulatip of 4-dimethylaminopyridine. Separation of the reaction mixture and concentration under reduced pressure gives 42.7 mg (67.2% of theory) of the product as a colourless solid.

HPLC (method A): R. =3.36 min.

MS (ESI pos): m/z=438 (M+H)⁺

¹H-NMR (400 MHz, CD₃CN): δ=1.06-1.35 (m, 4H), 1.62-2.46 (m, 7H{), 2.62-2.81 (m, 3H), 3.53-3.69 (m, 4H), 5.38 (d, 1H), 5.90 (br. s, 1H), 6.18 (m, 1H), 6.48 (m, 1H), 6.59 (br. s, 1H), 6.89 (m, 114), 7.23-7.48 (m, 5H), 8.68 (br. d, 1H), 9.82 (br. s, 1H).

### Example 19

(1R,2R)-N-[(1S)-2-Amino-2-oxo-1-phenylethyl]-2-{4-[cyclohexylcarbonyl]-1-piperazinyl}cyclohexanecarboxamide

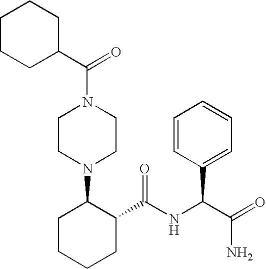

Analogously to the procedure for the preparation of Example 14, 50 mg (0.15 mmol) of the diastereomer 14b-A are reacted in DMF (2 ml) with 21.6 mg (0.16 mmol) of HOBt, 29.2 mg (0.15 mmol) of EDC, 22.3 mg (0.17 mmol) of cyclohexanecarboxylic acid, 0.06 ml (0.58 mmol) of 4-methylmorpholine and a spatulatip of 4-dimethylaminopyridine. Separation of the reaction mixture and concentration under reduced pressure gives 50.4 mg (76.4% of theory) of the product as a colourless solid.

HPLC (method A): R. =3.73 min.

MS (ESI pos): m/z=455 (M+H)⁺

¹H-NMR (400 MHz, CD₃CN): δ=1.06-1.42 (m, 9H), 1.55-2.20 (m, 9H), 2.25-2.42 (m, 3H), 2.43-2.56 (m, 1H), 2.57-2.76 (m, 3H), 3.26-3.45 (m, 4H), 5.35 (d, 1H), 5.85 (br. s, 1H), 6.55 (br. s, 1H), 7.25-7.47 (m, 5H), 8.72 (br. d, 1H).

### Example 20

(1R,2R)-N-[(1S)-2-Amino-2-oxo-1-phenylethyl]-2-[4-(carboxyphenyl)-1-piperazinyl]cyclohexanecarboxamide

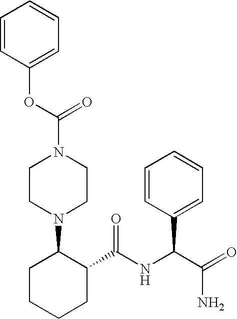

0.06 ml (0.44 mmol) of triethylamine is added to a solution of 50 mg (0.15 mmol) of the diastereomer 14b-A in methylene chloride (1.5 ml) and the mixture is cooled (ice cooling). A solution of 0.027 ml (0.22 mmol) of phenyl chloroformate in methylene chloride (0.5 ml) is then added, and the mixture is stirred with heating to RT for 2 h. The reaction mixture is extracted twice with in each case 10 ml of water and the organic phase is then dried over sodium sulphate, filtered and concentrated under reduced pressure. The residue is taken up in DMSO (5 ml) and the reaction mixture is then separated by preparative RP-HPLC (column: YMC Gel ODS-AQ S-11 μm, 250×30 mm; mobile phase: acetonitrile/water; flow rate: 50 ml/min; UV detection at 210 nm). This gives, after concentration under reduced pressure, 45.3 mg (67.2% of theory) of the product as a colourless solid.

HPLC (method A): Rₜ=3.88 min.

MS (ESI pos): m/z=465 (M+H)⁺

¹H-NMR (500 MHz, CD₃CN): δ=1.12-1.35 (m, 4H), 1.65-1.70 (m, 1H), 1.75-1.82 (m, 1H), 1.84-1.96 (m; 1H), 2.02-2.08 (m, 1H), 2.29-2.36 (m, 1H), 2.39-2.48 (m, 2H), 2.65-2.82 (m, 3H), 3.30-3.40 (m, 2H), 3.41-3.58 (m, 2H), 5.38 (d, 1H), 5.99 (br. s, 1H), 6.57 (br. s, 1H), 7.10 (d, 1H), 7.22 (t, 1H), 7.29-7.47 (m, 7H), 8.69 (d, 1H).

### Example 21

(1R,2R)-N-[(1S)-2-Amino-2-oxo 1-phenylethyl]-2-[4-(carboxycyclopentyl)-1-piperazinyl]cyclohexanecarboxamide

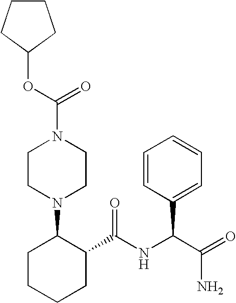

Analogously to the procedure for the preparation of the compound from Example 20, 50 mg (0.15 mmol) of the diastereomer 14b-A are reacted with 0.06 ml (0.44 mmol) of triethylamine and 0.031 ml (0.22 mmol) of cyclopentyl chlorocarbonate. Separation of the reaction mixture and concentration under reduced pressure gives 39.7 mg (59.9% of theory) of the product as a colourless solid.

HPLC (method A): Rₜ=3.93 min.

MS (ESI pos): m/z=457 (M+H)+

¹H-NMR (400 MHz, CD₃CN): δ=1.09-1.34 (m, 4H), 1.52-1.89 (m, 1H), 1.99-2.09 (m, 1H), 2.25-2.38 (m, 3H), 2.48-2.61 (m, 3H), 3.19-3.31 (m, 4H), 5.01 (m, 1H), 5.36 (d, 1H), 5.85 (br. s, 1H), 6.54 (br. s; 1H), 7.25-7.43 (m, 5H), 8.70 (d, 1H).

### Example 22

(1R,2R)-N-[(1S)-2-Amino-2-oxo-1-phenylethyl]-2-{4-[4-(acetylamino)benzyl]-1-piperazinyl}cyclohexanecarboxamide

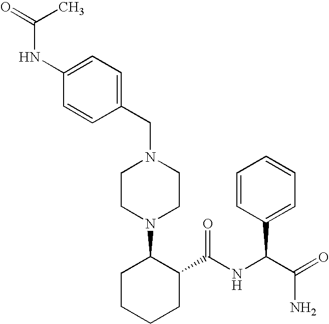

46.2 mg (0.22 mmol) of sodium triacetoxyborohydride are added to a solution of 50 mg (0.15 mmol) of the diastereomer 14b-A and 47.3 mg (0.29 mmol) of 4-acetamidobenzaldehyde in 1,2-dichlorethane (5 ml), 0.02 ml (0.29 mmol) of acetic acid is added dropwise and the mixture is stirred at RT for 2 h. Aqueous ammonia solution (10 ml) is added, and the organic phase is then separated off, dried over sodium sulphate, filtered and concentrated under reduced pressure. The residue is taken up in DMSO (5 ml) and the reaction mixture is then separated by preparative RP-HPLC (column: YMC Gel ODS-AQ S-11 μm, 250×30 mm; mobile phase: acetonitrile/water; flow rate: 50 ml/min; UV detection at 210 nm). This gives, after concentration under reduced pressure, 32 mg (43.5% of theory) of the product as a colourless solid.

HPLC (method A): Rₜ=3.31 min.

MS (ESI pos): In/z=492 (M+H)+

¹H-NMR (300 MHz, DMSO-d₆): δ=0.98-1.42 (m, 4H), 1.53-1.88 (m, 4H), 2.02 (s, 3H), 2.10-2.35 (m, 6H), 2.38-2.77 (m, 4H), 3.20-3.40 (m, 2H), 5.40 (d, 1H), 7.13 (m, 3H), 7.22-7.37 (m, 3H), 7.38-7.55 (m, 4H), 7.65 (m, 1H), 8.62 (d, 1H), 9.85 (s, 1H).

### Example 23

(1R,2R)-N-[(1S)-2-Amino-2-oxo-1-phenylethyl]-2-{4-[((4-trifluoromethyl)phenyl)-sulphonyl]-1-piperazinyl}cyclohexanecarboxamide

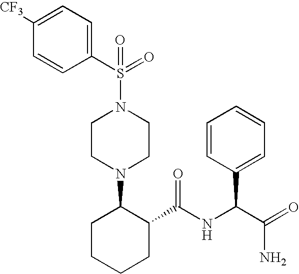

0.04 ml (0.29 mmol) of triethylamine is added to a solution of 50 mg (00.15 mmol) of the diastereomer 14b-A in methylene chloride (8 ml), and the mixture is cooled (ice cooling). A solution of 54.4 mg (0.22 mmol) of 4-(trifluoromethyl)benzenesulphonyl chloride in methylene chloride (2 ml) is then added, and the mixture is stirred with warming to RT for 3 h. Water (15 ml) and methylene chloride (5 ml) are added, the reaction mixture is extracted twice with in each case 10 ml of water and the organic phase is then dried over sodium sulphate, filtered and concentrated under reduced t pressure. The residue is taken up in DMSO (5 ml) and the reaction mixture is then separated by preparative RP-HPLC (column: YMC Gel ODS-AQ S-11 μm, 250×30 mm; mobile phase: acetonitrile/water; flow rate: 50 ml/min; Uv detection at 210 nm). This gives, after concentration under reduced pressure, 63.7 mg (79.4% of theory) of the product as a colourless solid.

HPLC (method A): Rₜ=4.19 min.

MS (ESI pos): in/z=553 (M+H)⁺

¹H-NMR (400 MHz, CD₃CN): δ=1.05-1.32 (m, 4H), 1.58-2.02 (m, 4H), 2.26 (m, 1H), 2.38-2.52 (m, 2H), 2.55-2.68 (m, 1H), 2.72-2.86 (m, 2H), 2.85-3.07 (m, 4H), 5.23 (d, 1H), 5.59 (br. s, 1H), 6.30 (br. s, 1H), 6.90-7.24 (m, 5H), 7.88-7.98 (m, 4H), 8.26 (d, 1H).

### Example 24

(1R,2R)-N-[(1S)-2-Amino-2-oxo-1-phenylethyl]-2-{4-[(4-fluorophenyl)amino-carbonyl]-1-piperazinyl}-cyclohexanecarboxamide

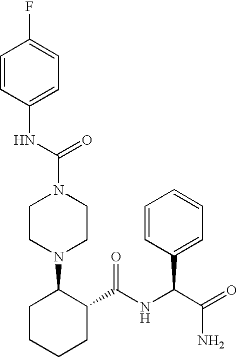

0.06 ml (0.44 mmol) of triethylamine and a spatulatip of 4-dimethylaminopyridine are added to a solution of 50 mg (0.15 mmol) of the diastereromer 14b-A in methylene chloride (1.5 ml), and the mixture is cooled (ice cooling). A solution of 0.02 ml (0.22 mmol) of 4-fluorobenzyl isocyanate in methylene chloride (0.5 ml) is then added, and the mixture is stirred with warming to RT for 8 h. Water (10 ml) and methylene chloride (10 ml) are added, the reaction mixture is extracted twice with in each case 10 ml of water and the organic phase is dried over sodium sulphate, filtered and concentrated under reduced pressure. The residue is taken up in DMSO (5 ml) and the reaction mixture is then separated by preparative RP-HPLC (column: YMC Gel ODS-AQ S-11 μm, 250×30 mm; mobile phase: acetonitrile/water; flow rate: 50 ml/min; UV detection at 210 nm). This gives, after concentration under reduced pressure, 19.9 mg (27.6% of theory) of the product as a colourless solid.

HPLC (method A): Rₜ=3.69 min.

MS (ESI pos): m/z=482 (M+H)⁺

¹H-NMR (400 MHz, CD₃CN): δ=1.02-1.35 (m, 4H), 1.59-2.23 (m, 4H), 2.24-2.47 (m, 3H), 2.60-2.80 (m, 3H), 3.22-3.40 (m, 4H), 5.36 (d, 1H), 5.85 (br. s, 1H), 6.55 (br. s, 1H), 6.95-7.18 (m, 3H), 7.23-7.48 (m, 7H), 8.73 (d, 1H).

### Example 25

Benzyl 4-[(1R,2R)-2-({[(1S)-2-amino-2-oxo-1-phenylethyl]amino}carbonyl)cyclo-hexyl]-1-piperazinecarboxylate 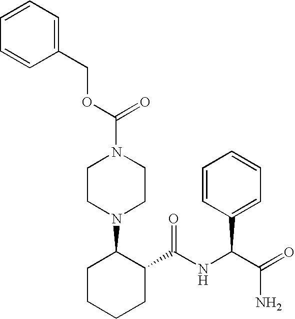
Step 25a):
Benzyl 4-[(1R*,2R*)-2-(tert-butoxycarbonyl)cyclohexyl]-1-piperazinecarboxylate

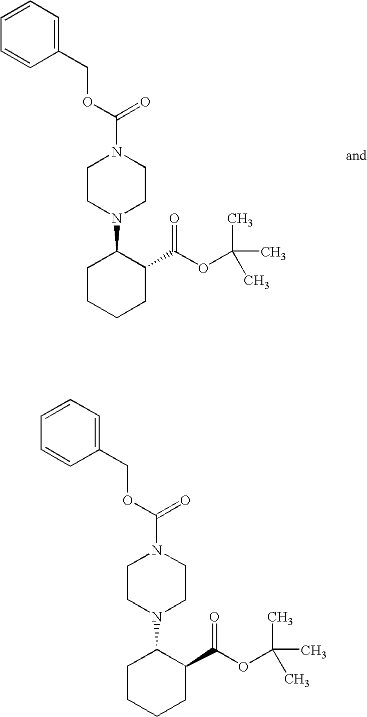

The product is prepared analogously to the compound from Example 1/step 1e) by reacting the compound of step 1d) using N,N-diisopropylethylamine as base and benzyloxycarbonyl chloride instead of benzoyl chloride. The product is obtained in a yield of 71% of theory

HPLC (method A): Rₜ=4.44 nin.

MS (ESI pos): m/z=403 (M+H)⁺.

Step 25b):

1-[(Benzyloxy)carbonyl]4-[(1R*,2R*)-2-carboxycyclohexyl]piperazin-4-ium trifluoroacetate

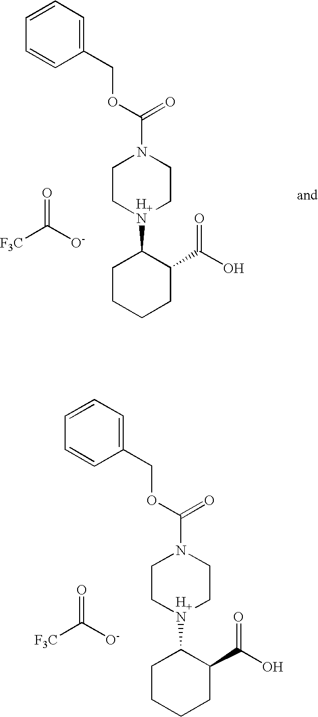

The compound from step 25a) is reacted analogously to Example 1/step 1f) with trifluoroacetic acid in dichloromethane. The crude product is twice taken up in ethyl acetate and reconcentrated to dryness. The product is obtained in a crude yield of 100%. It is reacted further without further purification.

HPLC (method A): Rₜ=3.80 min.

MS (ESI pos): m/z=347 (M+H)⁺.

Step 25c):

Diastereomer mixture of benzyl 4-[(1R,2R)-2-({[(1S)-2-amino-2-oxo-1-phenylethyl]amino}carbonyl)cyclo-hexyl]-1-piperazinecarboxylate and

benzyl 4-[(1S,2S)-2-({[(1S)-2-amino-2-oxo-1-phenylethyl]amino}carbonyl)cyclo-hexyl]-1-piperazinecarboxylate

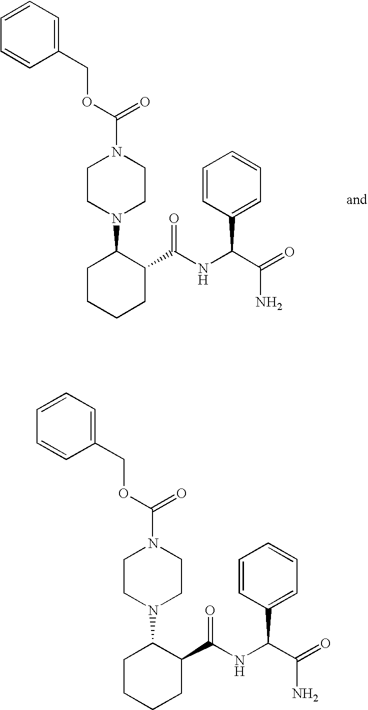

The compound from step 25b) is reacted analogously to Example 1/step 1g). This gives the mixture of diastereomers in a crude yield of 49%. It is directly separated into the two diastereomers using preparative HPLC.

Step 25d) (Separation of diastereomers):

Benzyl 4-[(1R,2R)-2-({[(1S)-2-amino-2-oxo-1-phenylethyl]amino}carbonyl)cyclo-hexyl]-1-piperazinecarboxylate (diastereomer 25A) 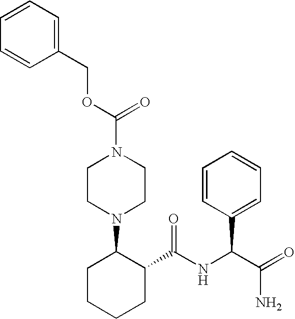
and
benzyl 4-[(1S,2S)-2-({[(1S)-2-amino-2-oxo 1-phenylethyl]amino}carbonyl)cyclo-hexyl]-1-piperazinecarboxylate (diastereomer 25B)

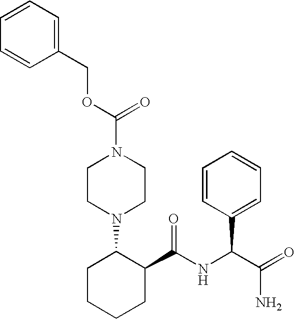

The mixture of diastereomers from step 25c) (1.0 g) is purified and separated into the diastereomers by preparative HPLC (Waters Symmetry RP 18-Phase, 7 μm, 19×300 mm, RT, injection volume=0.5 ml, flow rate=25 ml/min) using aqueous 0.2% strength trifluoroacetic acid (A)/acetonitrile (B) with the gradient 0 min 80% A/20% B→6 min 35% A/65% B→6.1 min 80% A/20% B→0.11 min 80% A/20% B. The two fractions are in each case taken up in dichloromethane, neutralized with aqueous sodium bicarbonate solution and extracted with dichloromethane, and the combined organic, phases are dried over sodium sulphate and filtered and the solvent is removed under reduced pressure. This gives 370 mg of diastereomer 25A and 330 mg of diastereomer 25B.

Diastereomer 25A:

HPLC (method A): Rₜ=3.94 min.

MS (ESI pos): m/z=479 (M+H)⁺

¹H-NMR (200 MHz, CDCl₃): δ=1.01-1.41 (br. m, 4H), 1.64-1.94 (br. m, 3H), 2.16-2.49 (br. m, 4H), 2.53-2.80 (br. m, 3H), 3.43 (m, 4H), 5.11 (s, 2H), 5.40 (br. s, 1H), 5.53 (d, 1H), 5.84 (br. s, 1H), 7.26-7.44 (m, 10H), 9.36 (d, 1H).

Diastereomer 25B:

HPLC (method A): Rₜ=4.08 min.

MS (ESI pos): m/z=479 (M+H)⁺

¹H-NMR (200 MHz, CDCl₃): δ=0.99-1.49 (br. m, 4H), 1.63-1.98 (br. m, 3H), 2.13-2.35 (br. m, 2H), 2.39-2.59 (br. m, 2H), 2.62-2.87 (br. m, 3H), 3.25-3.62 (br. m, 4H), 5.12 (s, 2H), 5.49 (br. s, 1H), 5.50 (d, 1H), 6.15 (br. s, 1H), 7.26-7.44 (m, 10H), 9.44 (br. d, 1H).

The Working Examples 26-162 listed in the table below are obtained analogously to the processes described above:

| Ex. | | | R₁ [min] | MS |
|---|---|---|---|---|
| No. | Structure | MW | (method) | [M + H]⁺ |
| 26 | 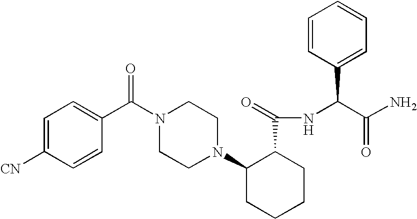 | 473.57 | 3.48 (A) | 474 |
| 27 | 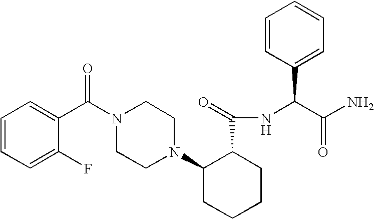 | 466.55 | 3.51 (A) | 467 |
| 28 | 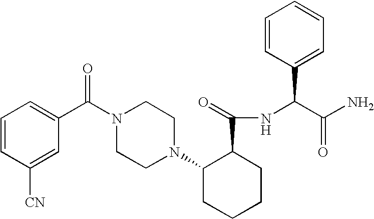 | 473.57 | 3.65 (A) | 474 |
| 29 | 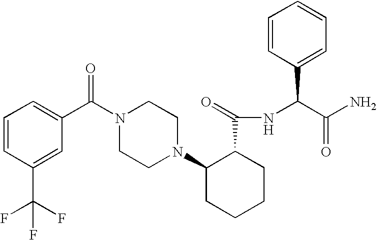 | 516.56 | 3.93 (A) | 517 |
| 30 | 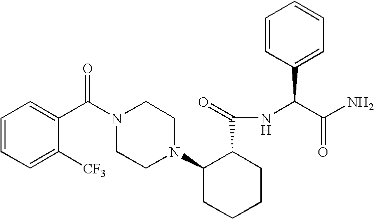 | 516.56 | 3.90 (A) | 517 |
| 31 | 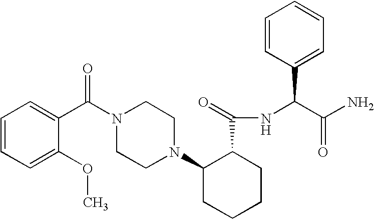 | 478.59 | 3.56 (A) | 479 |
| 32 | 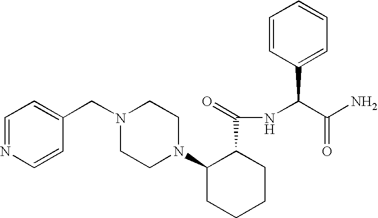 | 435.57 | 3.12 (A) | 436 |
| 33 | 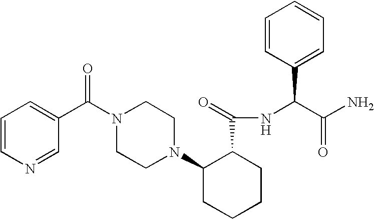 | 449.55 | 3.14 (A) | 450 |
| 34 | 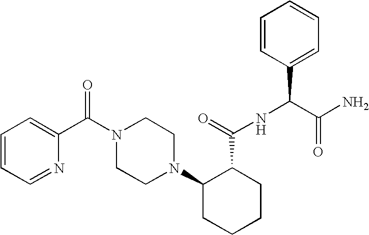 | 449.55 | 3.24 (A) | 450 |
| 35 | 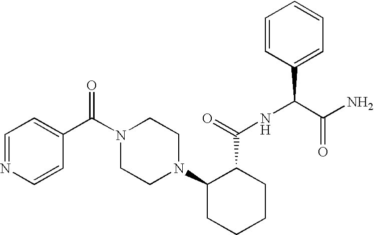 | 449.55 | 3.14 (A) | 450 |
| 36 | 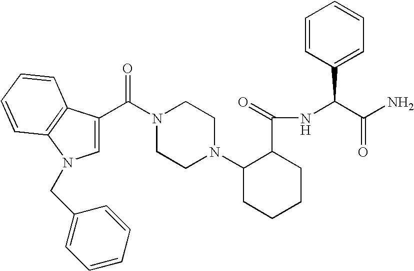 | 577.73 | 3.01 (E) | 578 |
| 37 | 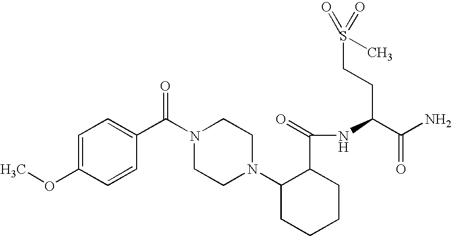 | 508.64 | 1.58/1.79 (E) | 509 |
| 38 | 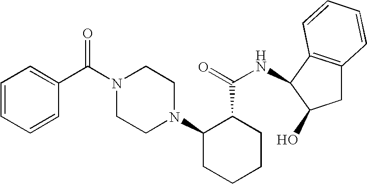 | 447.58 | 3.87 (A) | 448 |
| 39 | 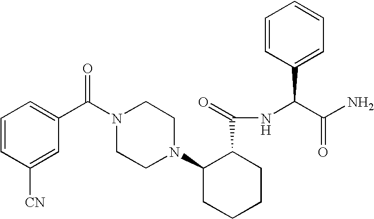 | 473.57 | 3.49 (A) | 474 |
| 40 | 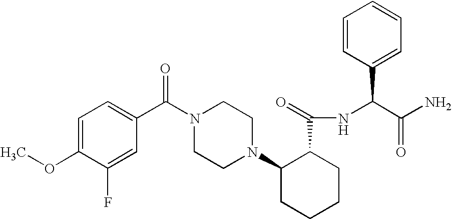 | 496.58 | 3.60 (A) | 497 |
| 41 | 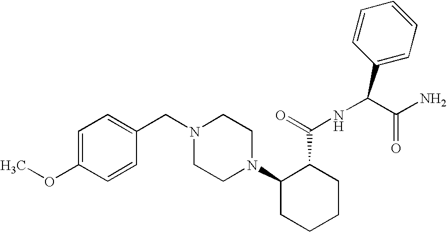 | 464.61 | 3.51 (A) | 465 |
| 42 | 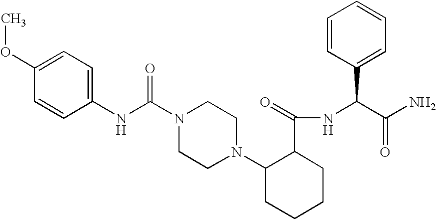 | 493.60 | 3.54/3.69 (A) | 497 |
| 43 | 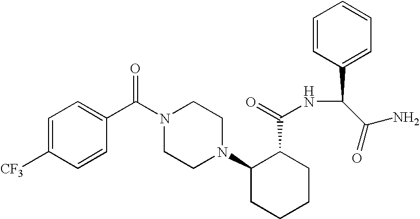 | 516.56 | 4.11 (A) | 517 |
| 44 | 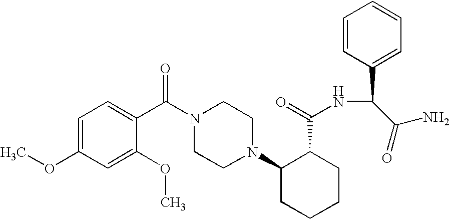 | 508.62 | 3.78 (A) | 509 |
| 45 | 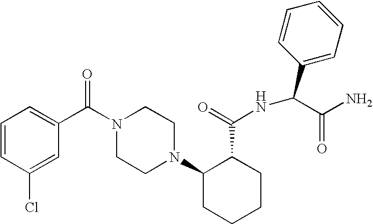 | 483.01 | 3.95 (A) | 483 |
| 46 | 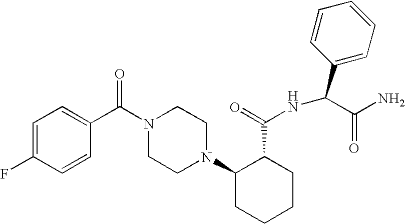 | 466.55 | 3.79 (A) | 467 |
| 47 | 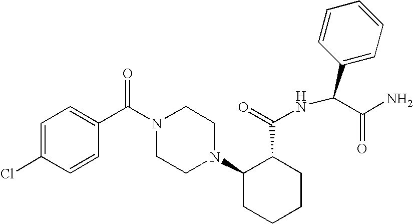 | 483.01 | 3.96 (A) | 483 |
| 48 | 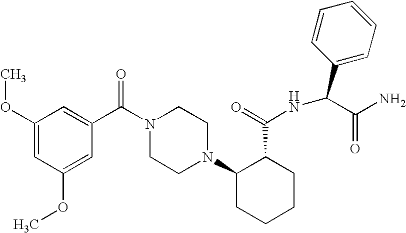 | 508.62 | 3.86 (A) | 509 |
| 49 | 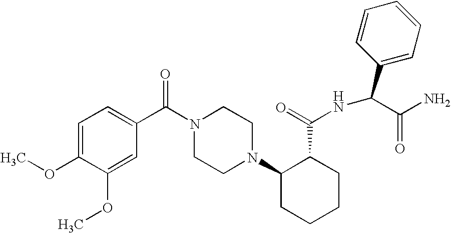 | 508.62 | 3.64 (A) | 509 |
| 50 | 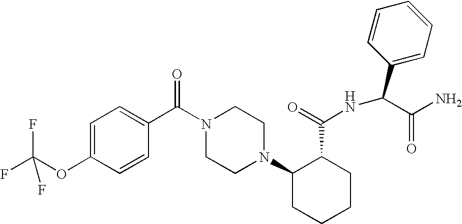 | 532.56 | 4.18 (A) | 533 |
| 51 | 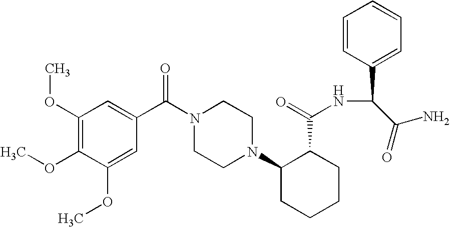 | 538.64 | 3.15 (E) | 539 |
| 52 | 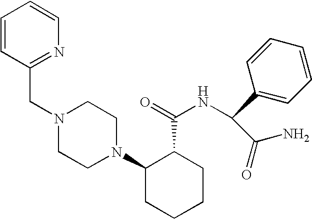 | 435.57 | 3.40 (A) | 436 |
| 53 | 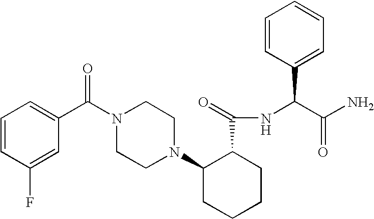 | 466.55 | 2.57 (F) | 467 |
| 54 | 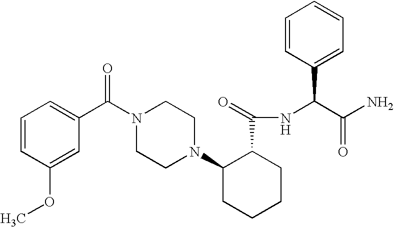 | 478.59 | 2.54 (F) | 479 |
| 55 | 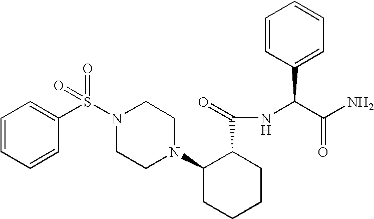 | 484.62 | 3.96 (A) | 485 |
| 56 | 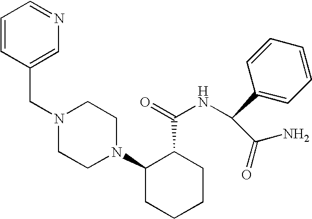 | 435.57 | 3.31 (A) | 436 |
| 57 | 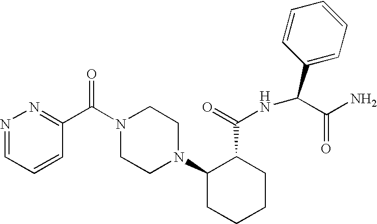 | 450.54 | 3.11 (A) | 451 |
| 58 | 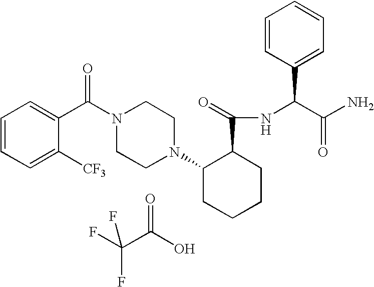 | 630.58 | 3.83 (A) | 517 |
| 59 | 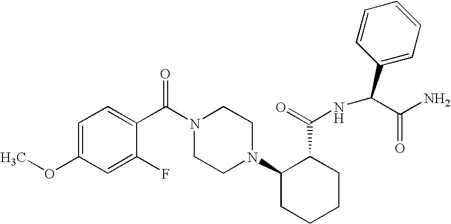 | 496.58 | 3.63 (A) | 497 |
| 60 | 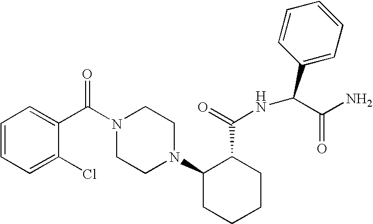 | 483.01 | 3.81 (A) | 483 |
| 61 | 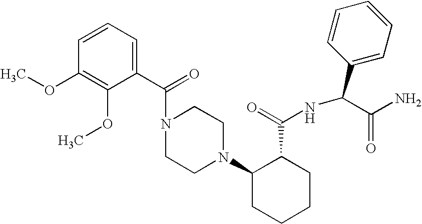 | 508.62 | 3.75 (A) | 509 |
| 62 | 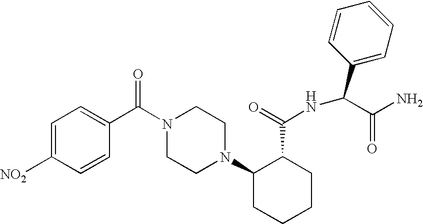 | 493.56 | 3.67 (A) | 494 |
| 63 | 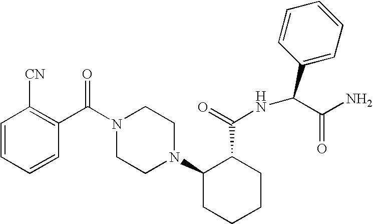 | 473.57 | 3.53 (A) | 474 |
| 64 | 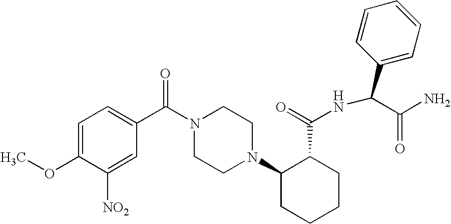 | 523.59 | 3.69 (A) | 524 |
| 65 | 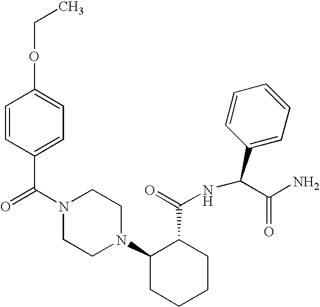 | 492.62 | 3.78 (A) | 493 |
| 66 | 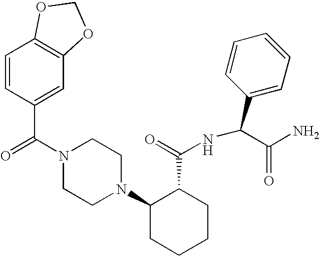 | 492.57 | 3.56 (A) | 493 |
| 67 | 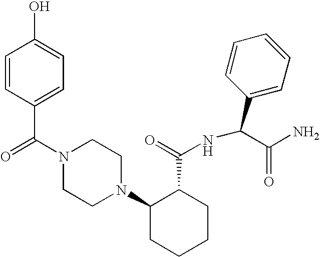 | 464.56 | 3.28 (A) | 465 |
| 68 | 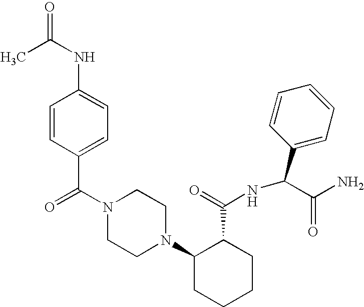 | 505.62 | 3.29 (A) | 506 |
| 69 | 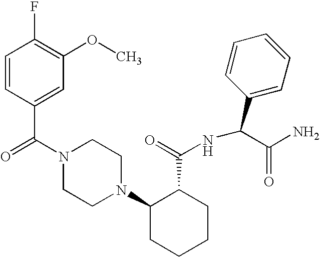 | 496.58 | 3.84 (A) | 497 |
| 70 | 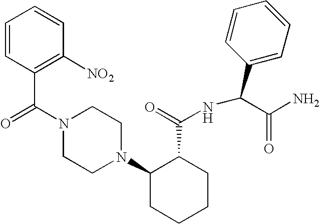 | 493.56 | 3.71 (A) | 494 |
| 71 | 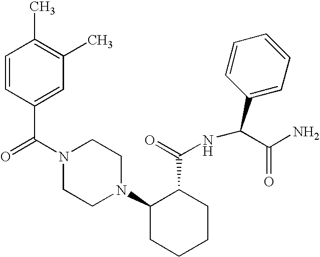 | 476.62 | 3.98 (A) | 477 |
| 72 | 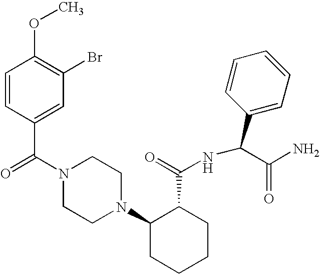 | 557.49 | 3.96 (A) | 559 |
| 73 | 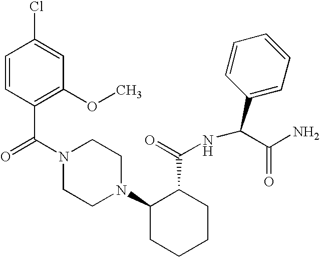 | 513.03 | 3.82 (A) | 513 |
| 74 | 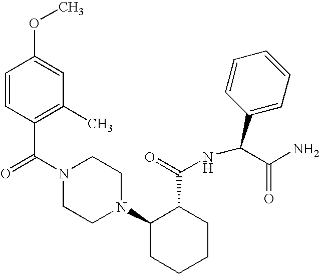 | 492.62 | 3.67 (A) | 493 |
| 75 | 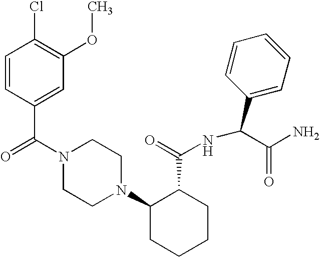 | 513.03 | 3.84 (A) | 513 |
| 76 | 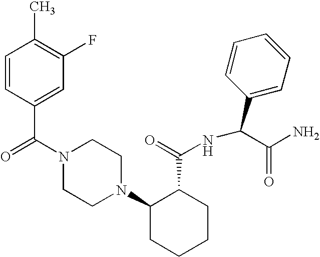 | 480.58 | 3.79 (A) | 481 |
| 77 | 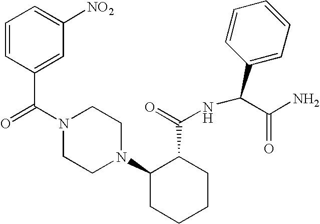 | 493.56 | 3.65 (A) | 494 |
| 78 | 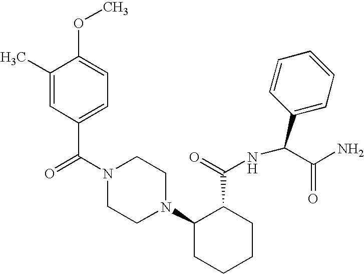 | 492.62 | 3.77 (A) | 493 |
| 79 | 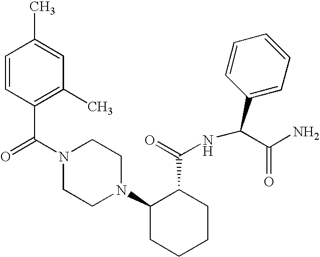 | 476.62 | 3.94 (A) | 477 |
| 80 | 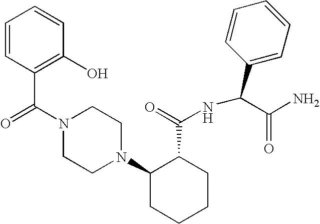 | 464.56 | 3.40 (A) | 465 |
| 81 | 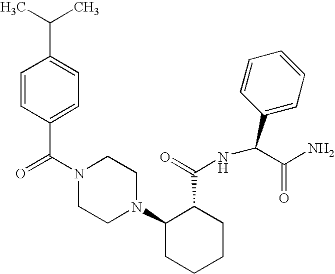 | 490.64 | 4.00 (A) | 491 |
| 82 | 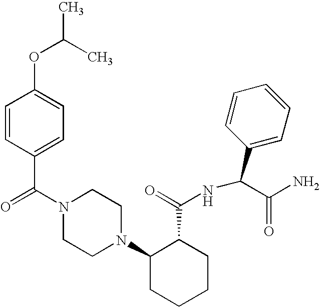 | 506.64 | 3.91 (A) | 507 |
| 83 | 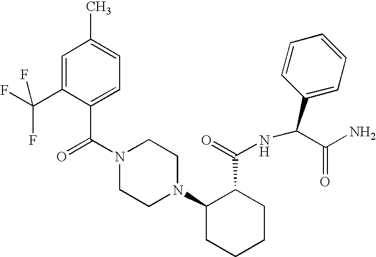 | 530.59 | 3.99 (A) | 531 |
| 84 | 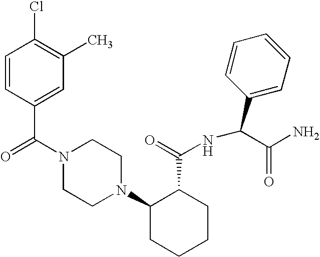 | 497.04 | 3.99 (A) | 497 |
| 85 | 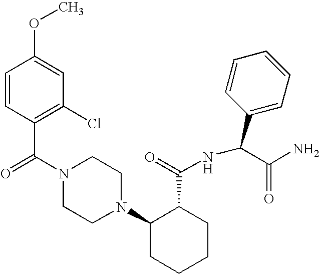 | 513.03 | 3.78 (A) | 513 |
| 86 | 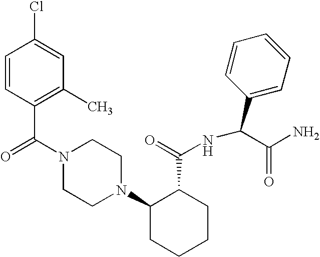 | 497.04 | 3.94 (A) | 497 |
| 87 | 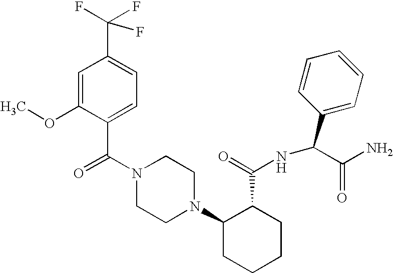 | 546.59 | 4.01 (A) | 547 |
| 88 | 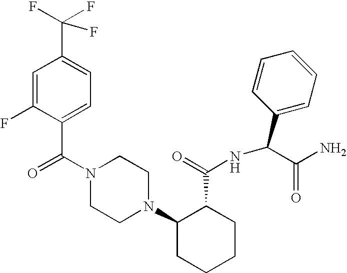 | 534.55 | 4.07 (A) | 535 |
| 89 | 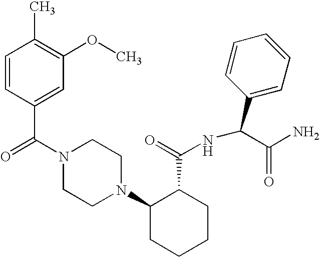 | 492.62 | 3.92 (A) | 493 |
| 90 | 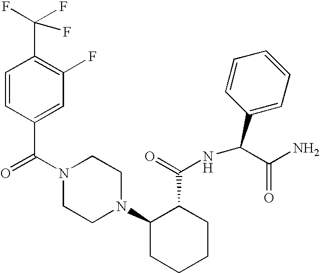 | 534.55 | 4.12 (A) | 535 |
| 91 | 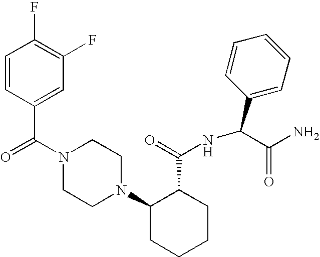 | 484.54 | 3.81 (A) | 485 |
| 92 | 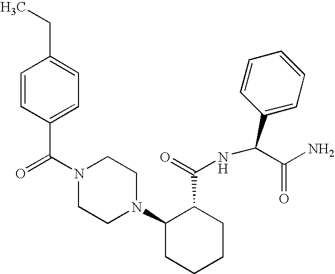 | 476.62 | 3.96 (A) | 477 |
| 93 | 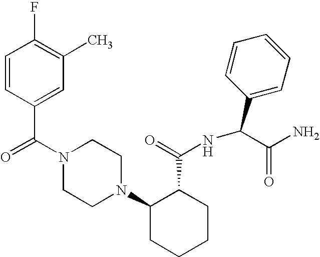 | 480.58 | 3.87 (A) | 481 |
| 94 | 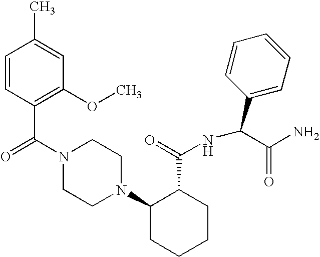 | 492.62 | 3.80 (A) | 493 |
| 95 | 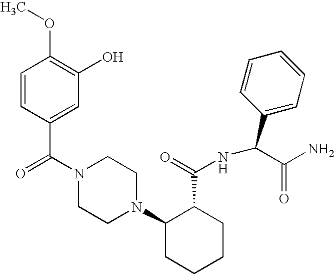 | 494.59 | 3.34 (A) | 495 |
| 96 | 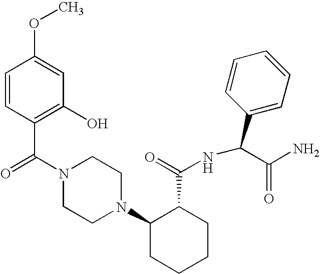 | 494.59 | 3.53 (A) | 495 |
| 97 | 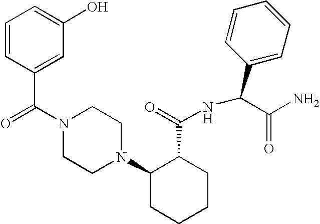 | 494.56 | 3.38 (A) | 465 |
| 98 | 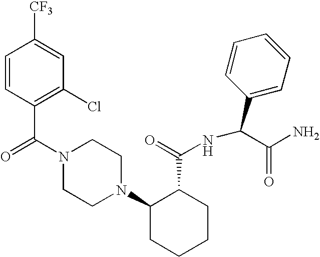 | 551.01 | 4.09 (A) | 551 |
| 99 | 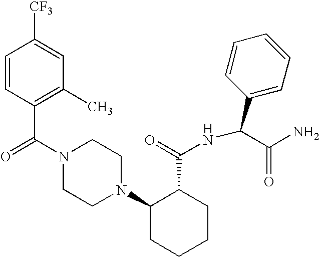 | 530.59 | 4.00 (A) | 531 |
| 100 | 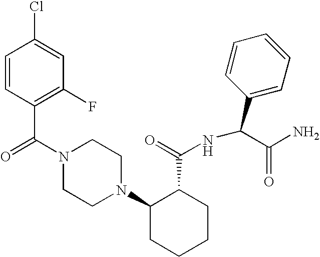 | 501.00 | 3.77 (A) | 501 |
| 101 | 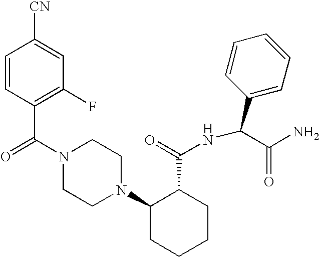 | 491.56 | 3.53 (A) | 492 |
| 102 | 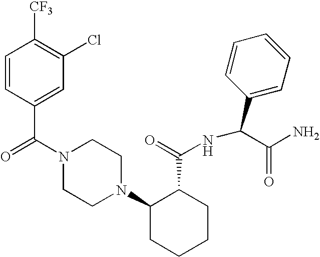 | 551.01 | 4.10 (A) | 551 |
| 103 | 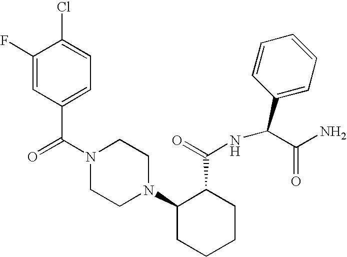 | 501.00 | 3.83 (A) | 501 |
| 104 | 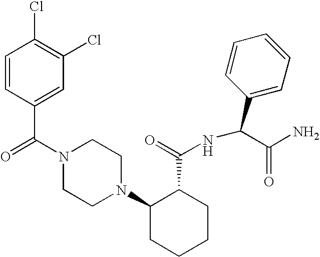 | 517.45 | 3.95 (A) | 517 |
| 105 | 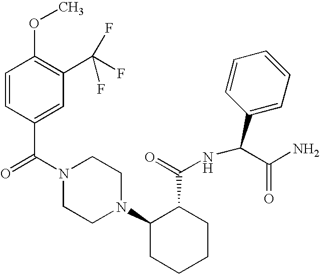 | 546.59 | 3.93 (A) | 547 |
| 106 | 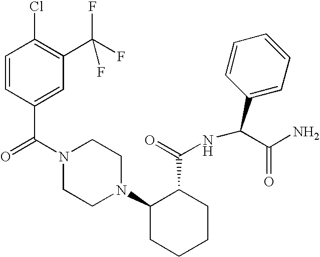 | 551.01 | 4.09 (A) | 551 |
| 107 | 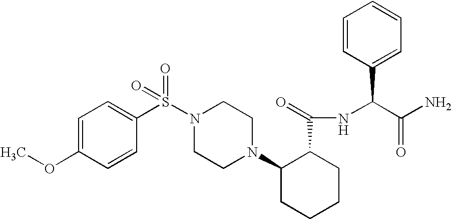 | 514.64 | 3.90 (A) | 515 |
| 108 | 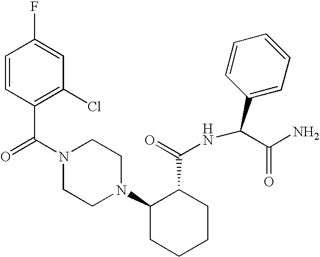 | 501.00 | 3.79 (A) | 501 |
| 109 | 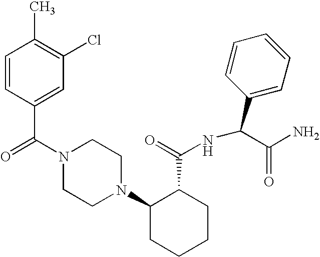 | 497.04 | 3.95 (A) | 497 |
| 110 | 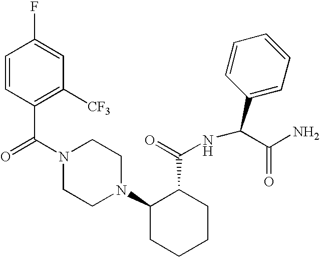 | 534.55 | 3.89 (A) | 535 |
| 111 | 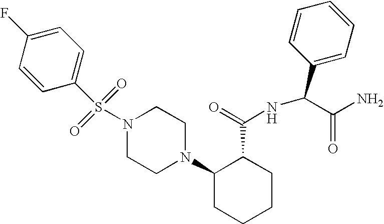 | 502.61 | 3.85 (A) | 503 |
| 112 | 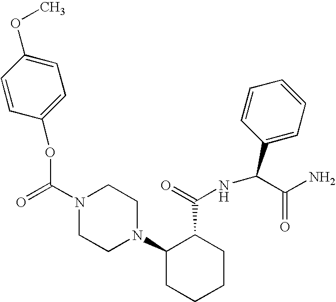 | 494.59 | 3.81 (A) | 495 |
| 113 | 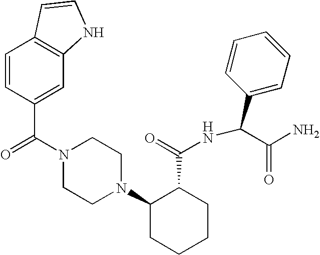 | 487.60 | 3.64 (A) | 488 |
| 114 | 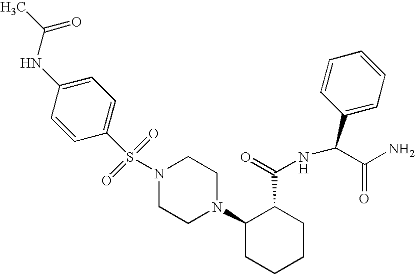 | 541.67 | 3.59 (A) | 542 |
| 115 | 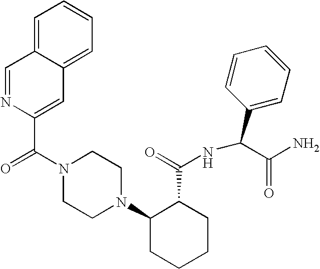 | 499.61 | 3.57 (A) | 500 |
| 116 | 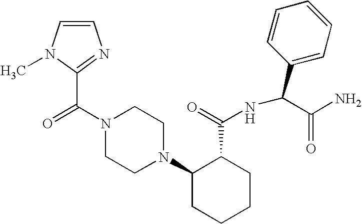 | 452.56 | 3.20 (A) | 453 |
| 117 | 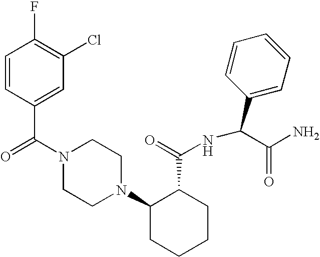 | 501.00 | 3.92 (A) | 501 |
| 118 | 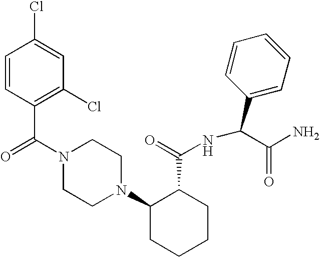 | 517.45 | 3.97 (A) | 517 |
| 119 | 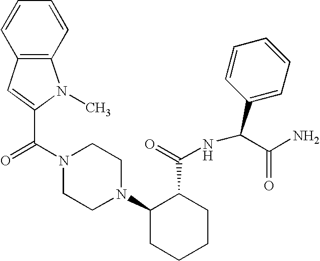 | 501.63 | 3.99 (A) | 502 |
| 120 | 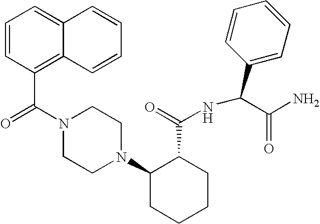 | 498.62 | 3.90 (A) | 499 |
| 121 | 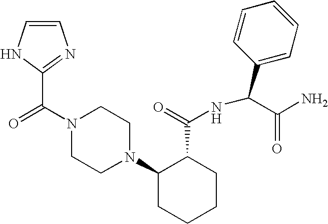 | 438.53 | 3.14 (A) | 439 |
| 122 | 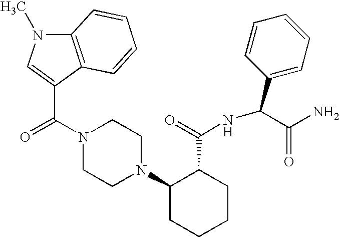 | 501.63 | 3.72 (A) | 502 |
| 123 | 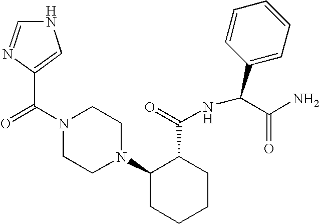 | 438.53 | 3.03 (A) | 439 |
| 124 | 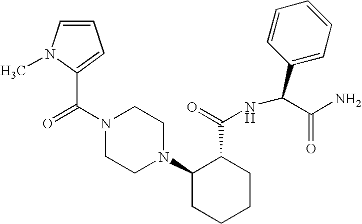 | 451.57 | 3.49 (A) | 452 |
| 125 | 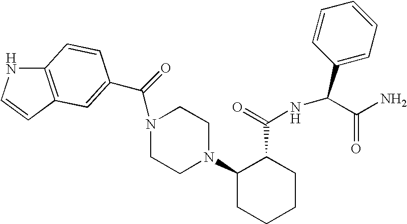 | 487.60 | 3.55 (A) | 488 |
| 126 | 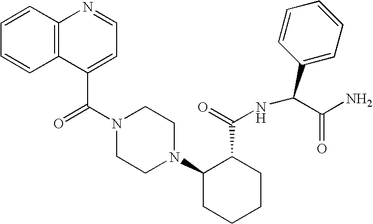 | 499.61 | 3.41 (A) | 500 |
| 127 | 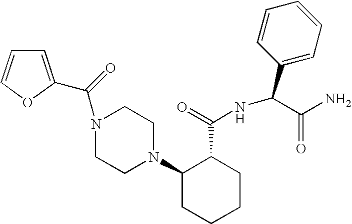 | 438.53 | 3.31 (A) | 439 |
| 128 | 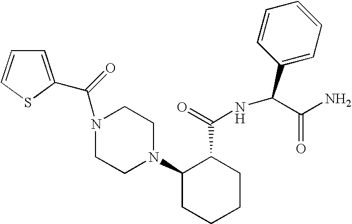 | 454.59 | 3.60 (A) | 455 |
| 129 | 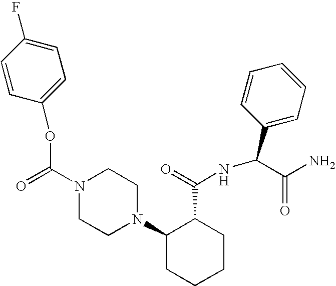 | 482.55 | 3.85 (A) | 483 |
| 130 | 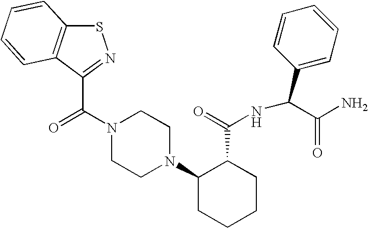 | 505.64 | 3.80 (A) | 506 |
| 131 | 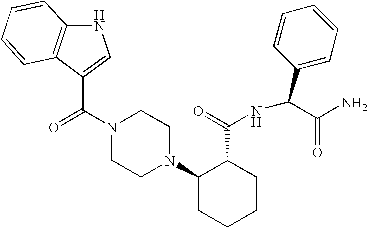 | 487.60 | 3.56 (A) | 488 |
| 132 | 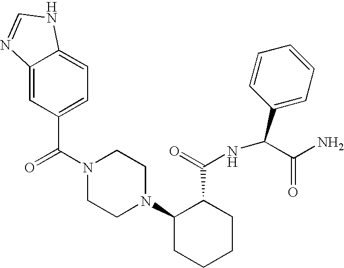 | 488.59 | 3.14 (A) | 489 |
| 133 | 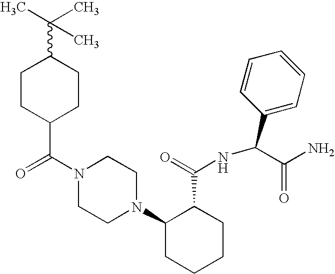 | 510.72 | 4.47 (A) | 511 |
| 134 | 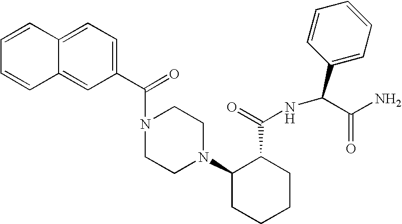 | 498.62 | 3.89 (A) | 499 |
| 135 | 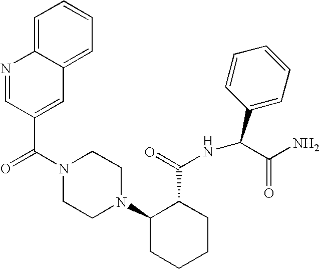 | 499.61 | 3.35 (A) | 500 |
| 136 | 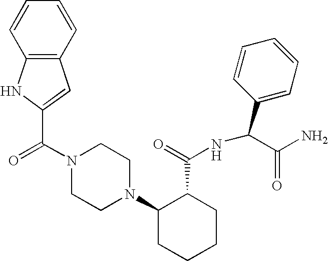 | 487.60 | 3.79 (A) | 488 |
| 137 | 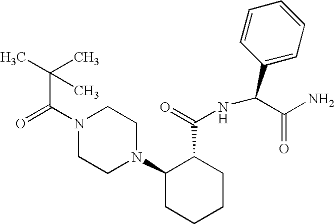 | 428.57 | 3.52 (A) | 429 |
| 138 | 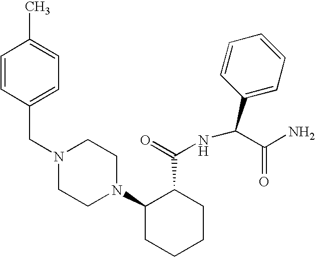 | 448.61 | 2.98 (E) | 449 |
| 139 | 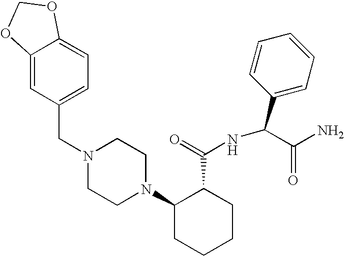 | 478.59 | 2.88 (E) | 479 |
| 140 | 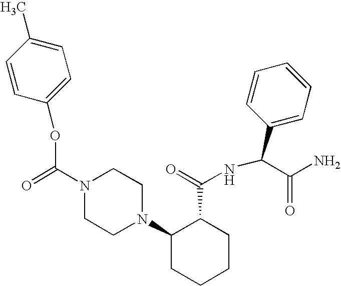 | 478.59 | 3.94 (A) | 479 |
| 141 | 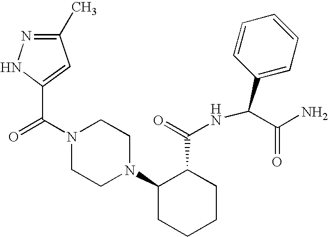 | 452.56 | 3.20 (A) | 453 |
| 142 | 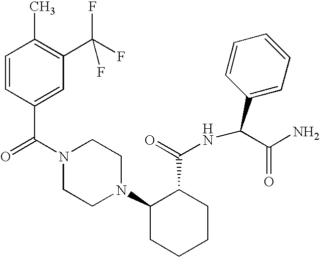 | 530.59 | 4.18 (A) | 531 |
| 143 | 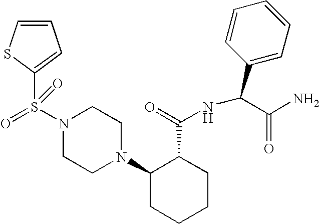 | 490.65 | 3.87 (A) | 491 |
| 144 | 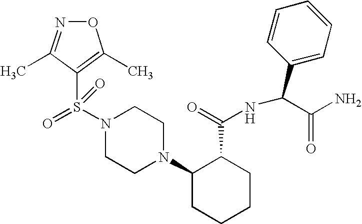 | 503.62 | 3.90 (A) | 504 |
| 145 | 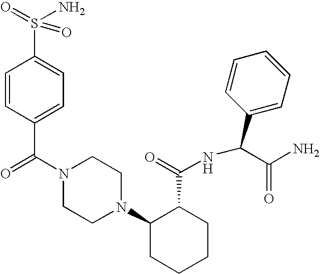 | 527.64 | 3.36 (A) | 528 |
| 146 | 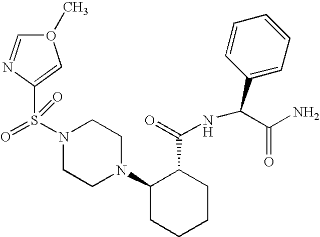 | 488.61 | 3.40 (A) | 489 |
| 147 | 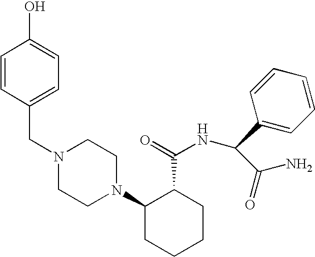 | 450.58 | 3.30 (A) | 451 |
| 148 | 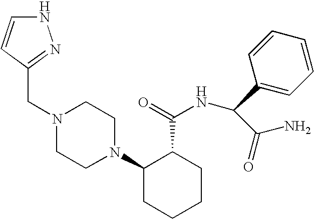 | 424.55 | 3.26 (A) | 425 |
| 149 | 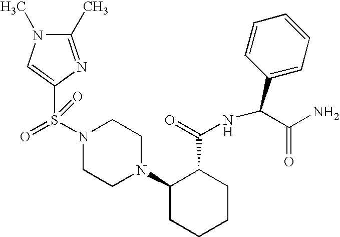 | 502.64 | 3.42 (A) | 503 |
| 150 | 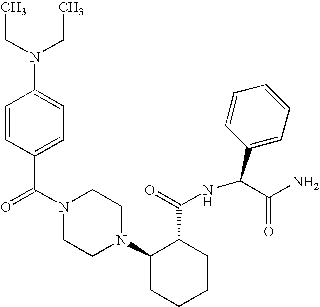 | 519.69 | 3.40 (A) | 520 |
| 151 | 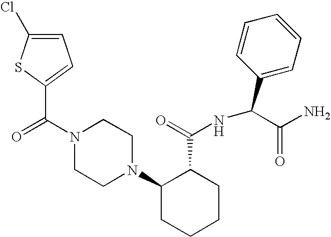 | 489.04 | 3.83 (A) | 489 |
| 152 | 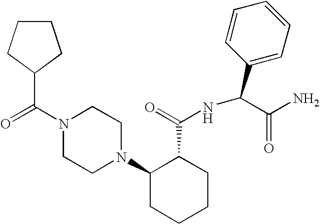 | 440.58 | 3.63 (A) | 441 |
| 153 | 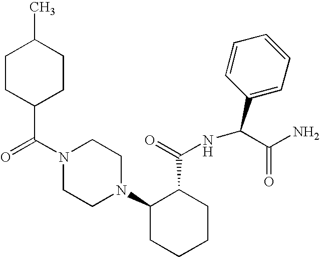 | 468.64 | 3.96 (A) | 469 |
| 154 | 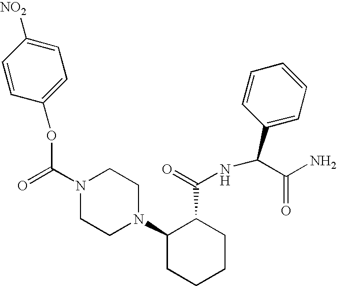 | 509.56 | 3.92 (A) | 510 |
| 155 | 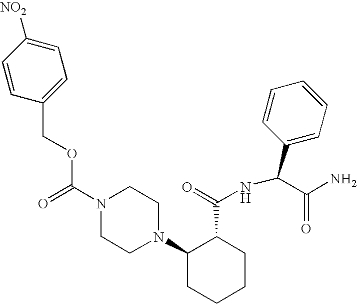 | 523.59 | 3.95 (A) | 524 |
| 156 | 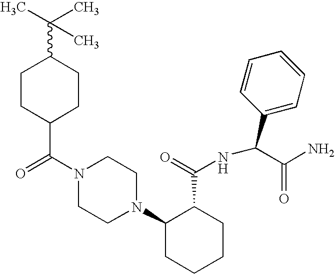 | 510.72 | 4.40 (A) | 511 |
| 157 | 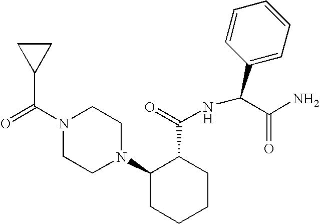 | 412.53 | 3.28 (A) | 413 |
| 158 | 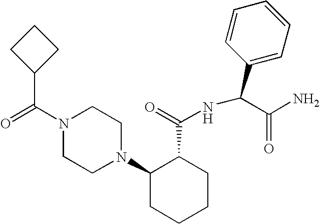 | 426.56 | 3.44 (A) | 427 |
| 159 | 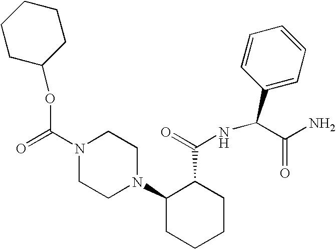 | 470.61 | 4.02 (A) | 471 |
| 160 | 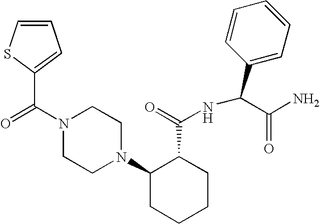 | 454.59 | 3.47 (A) | 455 |
| 161 | 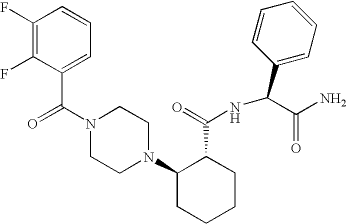 | 484.54 | 3.68 (A) | 485 |
| 162 | 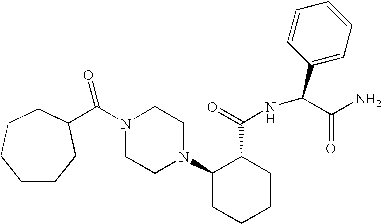 | 468.64 | 3.98 (A) | 469 |

The not commercially available aromatic carboxylic acids required for preparing various examples are described in the following literature references or can be prepared in an analogous manner:

### Example 57

Pyridazine-3-carboxylic acid; Leanza et al. J. Am. Chem. Soc. 1953, 75, 4086.

### Example 74

4-Methoxy-2-methylbenzoic acid; Mathur et al. J. Am. Chem. Soc. 1957, 79, 3582; Grethe et al. J. Org. Chem. 1968, 33, 494.

### Example 85

2-Chloro-4-methoxybenzoic acid; Noyce et al. J. Am. Chem. Soc. 1952, 74, 5144.

### Example 87

2-Methoxy-4-trifluoromethylbenzoic acid; McBee et al. J. Am. Chem. Soc. 1951, 73, 2375.

### Example 98

2-Chloro-4-trifluoromethylbenzoic acid; Mongin et al. Tetrahedron. Lett. 1996, 37, 2767.

### Example 99

Methyl 2-methyl-4-trifluoromethylbenzoate; Ueno et al. J. Med. Chem. 1976, 19, 941. The methyl ester can then be converted into the carboxylic acid using known methods (see, for example, in T. W. Greene, P. G. M. Wuts: Protective Groups in Organic Chemistry, 3rd Edition 1999, Wiley, New York).

### Example 101

2-Fluoro-4-cyanobenzoic acid; Fisher et al. Bioorg. Med. Chem. Lett. 2000, 10, 385.

### Example 102

3-Chloro-4-trifluoromethylbenzoic acid; preparation from 3-chloro-4-trifluoromethyltoluene analogously to Noyce et al. J. Am. Chem. Soc. 1952, 74, 5144.

### Example 121

1H-Imidazole-2-carboxylic acid; Curtis et al. J. Org. Chem. 1980, 45, 4038.

### Example 130

Benzo[d]isothiazole-3-carboxylic acid; Clarke et al. J. Chem. Res. Miniprint 1979, 4677; Stolle Chem. Ber. 1925, 58, 2096.

### Example 141

5-Methylpyrazolecarboxylic acid; Rojahn Chem. Ber. 1926, 59, 609, Knorr et al. Liebigs Ann. Chem. 1894, 279, 217.

HPLC methods

| Method A: | column: | Kromasil C18 60 × 2 mm |
|---|---|---|
| | mobile phase: | A = 0.5% HClO₄ in water |
| | | B = acetonitrile |
| | gradient: | 0.0-0.5 min 98% A |
| | | 4.5-6.5 min 10% A |
| | | 6.7-7.5 min 98% A |
| | flow rate: | 0.75 ml/min |
| | temp.: | 30° C. |
| | detection: | 210 nm |
| Method B: | column: | Kromasil 100 C18 125 × 4 mm |
| | mobile phase: | A = 1.0% HClO₄ in water |
| | | B = acetonitrile |
| | gradient: | 0.0-0.5 min 98% A |
| | | 4.5-6.5 min 10% A |
| | | 6.7-7.5 min 98% A |
| | flow rate: | 0.75 ml/min |
| | temp.: | 30° C. |
| | detection: | 210 nm |
| Method C: | column: | Kromasil C18 60 × 2 mm |
| | mobile phase: | A = H₃PO₄ 0.01 mol/l |
| | | B = acetonitrile |
| | gradient: | 0.0-0.5 min 90% A |
| | | 4.5-6.5 min 10% A |
| | | 7.5 min 90% A |
| | flow rate: | 0.75 ml/min |
| | temp.: | 30° C. |
| | detection: | 210 nm |
| Method D: | analogous to method A, but using | |
| | gradient: | 0.0-0.5 min 98% A |
| | | 4.5-6.5 min 10% A |
| | | 9.2 min 98% A |
| Method E: | column: | Symmetry C18 50 × 2.1 mm |
| | mobile phase: | A = 0.1% formic acid in water |
| | | B = 0.1% formic acid in acetonitrile |
| | gradient: | 0.0-4 min 90% A |
| | | 4-6.1 min 10% A |
| | | 6.1-7.5 min 90% A |
| | flow rate: | 0.5 ml/min |
| | temp.: | 40° C. |
| | detection: | 210 nm |
| Method F: | analogous to method E, but using: | |
| | gradient: | 0.0-5 min 95% A |
| | | 5-6 min 10.% A |
| | | 6-7.5 min 90% A |
| | flow rate: | 1 ml/min |
| | temp.: | 50° C. |

1. A compound Compounds of the formula (I) 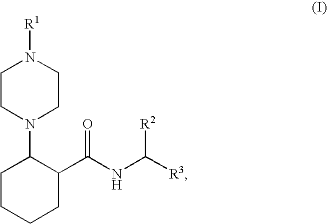
in which
R¹ represents a group of the formula *C(═O)—R⁴, *(CH₂)ₐ—R⁴, *SO₂—R⁴, *C(═O)—NR⁵R⁶ or *C(═O)—OR⁷,
in which
* represents the point of attachment,
a represents 0, 1, 2 or 3,
R⁴ represents (C₁-C₆)-alkyl, (C₃-C₈)-cycloalkyl, which is optionally substituted by (C₁-C₆)-alkyl or hydroxyl, (C₆-C₁₀)-aryl or 5- to 10-membered heteroaryl having up to three heteroatoms from the group consisting of N, O and S,
where aryl and heteroaryl for their part may be substituted up to three times independently of one another by halogen, trifluoromethyl, trifluoromethoxy, cyano, carboxyl, nitro, hydroxyl, sulphamoyl, (C₁-C₆)-alkoxy, (C₁-C₆)-alkoxycarbonyl, amino, mono- or di-(C₁-C₆)-alkylamino, (C₁-C₄)-alkylcarbonylamino, (C₃-C₈)-cycloalkyl, (C₆-C₁₀)-aryl, 5- or 6-membered heteroaryl having up to three heteroatoms from the group consisting of N, O and S, 5- to 7-membered heterocyclyl having up to two heteroatoms from the group consisting of N, O and S, where N is substituted by hydrogen, (C₁-C₄)-alkyl or (C₃-C₇)-cycloalkyl, or
(C₁-C₆)-alkyl, whose chain may be interrupted by an oxygen atom or a sulphur atom or by an NH group and which for its part may be substituted by hydroxyl, mono- or di-(C₁-C₆)-alkylamino, phenyl or 5- to 7-membered heterocyclyl having up to two heteroatoms from the group consisting of N, O and S, where N is substituted by hydrogen, (C₁-C₄)-alkyl or (C₃-C₇)-cycloalkyl,
R⁵ and R⁶ independently of one another represent hydrogen, (C₆-C₁₀)-aryl or 5- to 10-membered heteroaryl having up to three heteroatoms from the group consisting of N, O and S, where aryl and heteroaryl for their part may be substituted up to three times independently of one another by halogen, trifluoromethyl, trifluoromethoxy, cyano, nitro, hydroxyl, amino, (C₁-C₆)-alkyl or (C₁-C₆)-alkoxy,
adamantyl, (C₁-C₈)-alkyl, whose chain may be interrupted by one or two oxygen atoms and which may be substituted up to three times independently of one another by hydroxyl, phenyl, trifluoromethyl, (C₃-C₈)-cycloalkyl, (C₁-C₆)-alkoxy, mono- or di-(C₁-C₆)-alkylamino, 5- or 6-membered heterocyclyl having up to three heteroatoms from the group consisting of N, O and S or by 5- to 10-membered heteroaryl having up to three heteroatoms from the group consisting of N, O and S, (C₃-C₈)-cycloalkyl, which may be substituted up to three times independently of one another by (C₁-C₄)-alkyl, hydroxyl or oxo, or
5- or 6-membered heterocyclyl having up to two heteroatoms from the group consisting of N, O and S, where N is substituted by hydrogen or (C₁-C₄)-alkyl,
or
R⁵ and R⁶ together with the nitrogen atom to which they are attached form a 4- to 7-membered saturated heterocycle which may contain up to two further heteroatoms from the group consisting of N, O and S and which is optionally substituted by hydroxyl, oxo or (C₁-C₆)-alkyl which for its part may be substituted by hydroxyl,
R⁷ represents (C₆-C₁₀)-aryl or 5- to 10-membered heteroaryl having up to three heteroatoms from the group consisting of N, O and S, where aryl and heteroaryl for their part may be substituted up to three times independently of one another by halogen, trifluoromethyl, trifluoromethoxy, cyano, nitro, hydroxyl, amino, (C₁-C₆)-alkyl or (C₁-C₆)-alkoxy,
adamantyl, (C₁-C₈)-alkyl whose chain may be interrupted by one or two oxygen atoms and which may be substituted up to three times independently of one another by hydroxyl, phenyl which for its part may be substituted by nitro, halogen, trifluoromethyl, trifluoromethoxy, (C₁-C₆)-alkyl or cyano, trifluoromethyl, (C₃-C₈)-cycloalkyl, (C₁-C₆)-alkoxy, mono- or di-(C₁-C₆)-alkylamino, 5- or 6-membered heterocyclyl having up to three heteroatoms from the group consisting of N, O and S or by 5- to 10-membered heteroaryl having up to three heteroatoms from the group consisting of N, O and S,
(C₃-C₈)-cycloalkyl which may be substituted up to three times independently of one another by (C₁-C₄)-alkyl, hydroxyl or oxo, or
5- or 6-membered heterocyclyl having up to two heteroatoms from the group consisting of N, O and S, where N is substituted by hydrogen or (C₁-C₄)-alkyl,
R² represents (C₁-C₈)-alkyl whose chain may be interrupted by a sulphur atom or oxygen atom or by an S(O) or SO₂ group, phenyl, benzyl or 5- or 6-membered heteroaryl having up to two heteroatoms from the group consisting of N, O and S, where phenyl, benzyl and heteroaryl for their part may be substituted up to three times independently of one another by halogen, trifluoromethyl, trifluoromethoxy, cyano, nitro, hydroxyl, amino, (C₁-C₆)-alkyl or (C₁-C₆)-alkoxy, and
R³ represents a group of the formula *CH₂—OH or *C(O)—NR⁸R⁹,
in which
* represents the point of attachment,
R⁸ and R⁹ independently of one another represent hydrogen or (C₁-C₆)-alkyl, or
R² and R³ together with the CH group to which they are attached represent a group of the formula 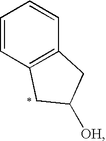
in which
* represents the point of attachment,
or its salt hydrate, hydrate of a salt or solvent.

2. A compound of the formula (I) according to claim 1,
in which
R¹ represents a group of the formula *C(═O)—R⁴, *(CH₂)ₐ—R⁴ or *C(═O)—OR⁷,
in which
* represents the point of attachment,
a represents 1,
R⁴ represents (C₆-C₁₀)-aryl or 5- to 10-membered heteroaryl having up to three heteroatoms from the group consisting of N, O and S, where aryl and heteroaryl may be substituted up to three times independently of one another by halogen, trifluoromethyl, trifluoromethoxy, cyano, nitro, hydroxyl, amino, (C₁-C₆)-alkyl, (C₁-C₆)-alkylcarbonylamino or (C₁-C₆)-alkoxy,
R⁷ represents phenyl which may be substituted up to three times independently of one another by halogen, trifluoromethyl, trifluoromethoxy, cyano, nitro, hydroxyl, amino, (C₁-C₆)-alkyl or (C₁-C₆)— alkoxy,
methyl which may be substituted by phenyl or (C₃-C₈)-cycloalkyl, or (C₃-C₈)-cycloalkyl,
R² represents phenyl, benzyl or 5- or 6-membered heteroaryl having up to two heteroatoms from the group consisting of N, O and S, where phenyl, benzyl and heteroaryl for their part may be substituted up to three times independently of one another by halogen, hydroxyl, amino, (C₁-C₄)-alkyl or (C₁-C₄)-alkoxy, and
R³ represents a group of the formula *C(O)—NR⁸R⁹,
in which
* represents the point of attachment,
R⁸ and R⁹ independently of one another represent hydrogen, methyl or ethyl,
or its salt, hydrate, hydrate of a salt or solvate.

3. A compound of the formula (I) according to claim 1,
in which
R¹ represents a group of the formula *C(═O)—R⁴ or *(CH₂)ₐ—R⁴,
in which
* represents the point of attachment,
a represents 1,
R⁴ represents (C₆-C₁₀)-aryl or 5- to 10-membered heteroaryl having up to three heteroatoms from the group consisting of N, O and S, where aryl and heteroaryl may be substituted up to three times independently of one another by halogen, trifluoromethyl, trifluoromethoxy, cyano, nitro, hydroxyl, amino, (C₁-C₆)-alkyl, (C₁-C₆)-alkylcarbonylamino or (C₁-C₆)-alkoxy,
R² represents phenyl, benzyl or 5- or 6-membered heteroaryl having up to two heteroatoms from the group consisting of N, O and S, where phenyl, benzyl and heteroaryl for their part may be substituted up to three times independently of one another by halogen, hydroxyl, amino, (C₁-C₄)-alkyl or (C₁-C₄)-alkoxy,
and
R³ represents a group of the formula *C(O)—NR⁸R⁹,
in which
* represents the point of attachment,
R⁸ and R⁹ independently of one another represent hydrogen or methyl,
or its salt, hydrate, hydrate of a salt or solvate.

4. A compound of the formula (I) according to claim 1,
in which
R¹ represents a group of the formula *C(═O)—R⁴,
in which
*represents the point of attachment,
R⁴ represents phenyl, naphthyl, indolyl, indazolyl, benzimidazolyl, benzisothiazolyl, pyrrolyl, furyl, thienyl, quinolinyl, isoquinolinyl, pyrazolyl, piperonyl, pyridinyl, pyrazinyl or pyridazinyl which for their part may be substituted up to two times independently of one another by fluorine, chlorine, trifluoromethyl, trifluoromethoxy, cyano, nitro, hydroxyl, acetylamino, methyl, ethyl, n-propyl, isopropyl, methoxy, ethoxy, n-propoxy or isopropoxy,
R² represents phenyl which may optionally be substituted by fluorine in the para position to the point of attachment, or pyridyl,
and
R³ represents a group of the formula *C(O)—NR⁸R⁹,
in which
* represents the point of attachment,
R⁸ and R⁹ represent hydrogen,
or its salt hydrate, hydrate of a salt or solvate.

5. A compound according to claim 1 having the following strucure:
(1R,2R)-N-[(1S)-2-amino-2-oxo-1-phenylethyl]-2-(4-benzoyl-1-piperazinyl)cyclohexanecarboxamide 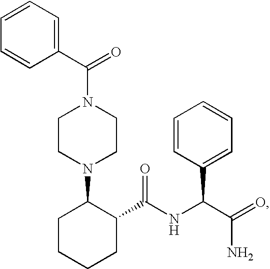
(1R,2R)-N-[(1S)-2-amino-2-oxo-1-(4-fluorophenyl)ethyl]-2-(4-benzoyl-1-piperazinyl)cyclohexanecarboxamide 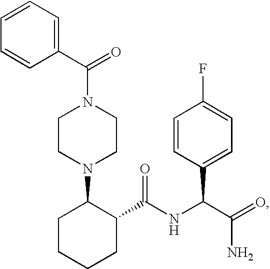
(1R,2R)-N-[(1S)-2-amino-2-oxo-1-phenylethyl]-2-[4-(1H-indazol-3-ylcarbonyl)-1-piperazinyl]cyclohexanecarboxamide 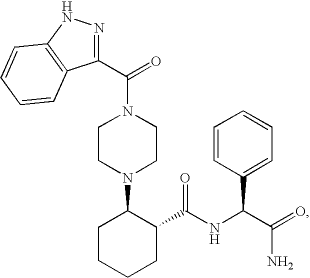
(1R,2R)-N-[(1S)-2-amino-2-oxo-1-phenylethyl]-2-[4-(2,4-difluorobenzoyl)-1-piperazinyl)cyclohexanecarboxamide 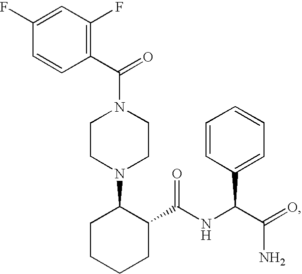
(1R,2R)-N-[(1S)-2-amino-2-oxo-1-phenylethyl]-2-{4-[(5-methyl-2-thienyl)carbonyl]-1-piperazinyl}cyclohexanecarboxamide 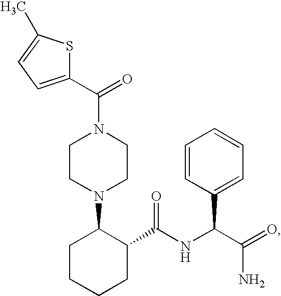
(1R,2R)-N-[(1S)-2-amino-2-oxo-1-phenylethyl]-2-{4-[(2-pyrrolyl)carbonyl]-1-piperazinyl}cyclohexanecarboxamide 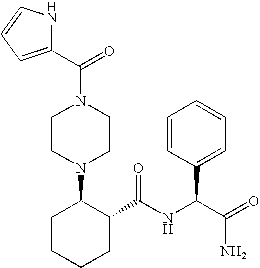
or its salt, hydrate, hydrate of a salt or solvate.

6. A compound of the formula (I), as defined in claim 1, characterized by one of the following stereochemical configurations according to formulae (Ia) to (Id):

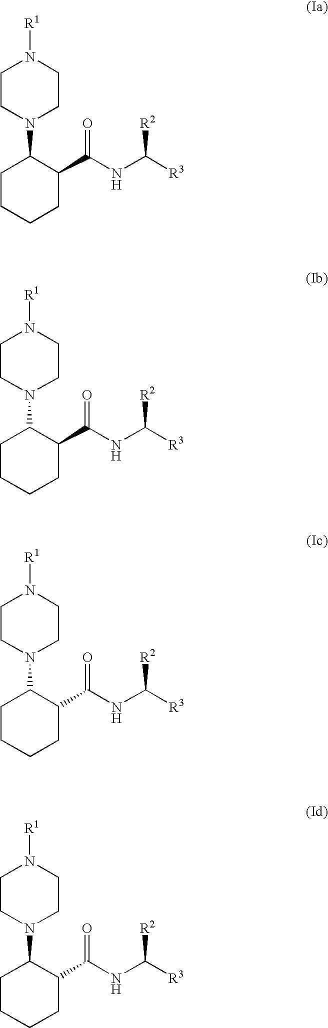

7. A compound of the formula (I) according to claim 6, characterized by the following stereochemical configuration according to formula (Id):

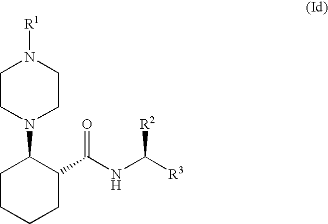

8. Process for preparing compounds of the formula (I) as defined in claim 1, characterized in that
[A] compounds of the formula (II) 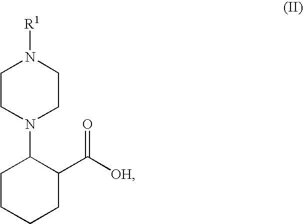
in which
R¹ is as defined in claim 1,
are reacted with compounds of the formula (III) 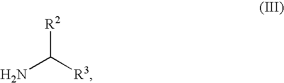
in which
R² and R³ are as defined in claim 1,
or
[B] compounds of the formula (IV) 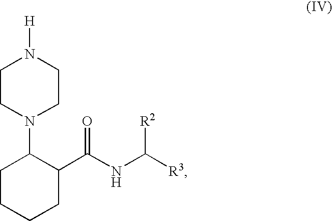
in which
R² and R³ are as defined in claim 1,
are reacted with compounds of the formula (V), (Va) or (Vb)
R¹—X (V),
R⁵R⁶ N═C═O (Va),
R⁴—(CH2)_{a—1}—CHO (Vb),
in which
R¹, R⁵, R⁶ are as defined above,
a represents 1, 2, or 3 and
X represents a leaving group or represents a hydroxyl group.

9. (canceled)

10. A pharmaceutical compostion, comprising at least one compound of the formula (I) as defined in claim 1 and at least one auxiliary.

11. A pharmaceutical compostion, comprising at least one compound of the formula (I) as defined in claim 1 and at least one further active compound.

12. A method of treating or preventing ischaemia-related peripheral and cardiovascular disorders, comprising administering to a patient in need thereof an effective amount of a compound of formula (I) as defined in claim 1.

13. A method of treating or preventing acute and chronic ischaemic disorders of the cardiovascular system, comprising administering to a patient in need thereof an effective amount of a compound of formula (I) as defined in claim 1.

14. A method of treating or preventing coronary heart disease, stable and unstable angina pectoris, peripheral and arterial occlusion diseases, thrombotic vascular occlusions, myocardial infarction or reperfusion damage, comprising administering to a patient in need thereof an effective amount of a compound of formula (I) as defined in claim 1.
